(19) United States
(12) Patent Application Publication
(10) Pub. No.: US 2026/0256586 A1
(43) Pub. Date: Sep. 3, 2026

(54) CARDIAC LEAFLET COAPTERS

(71) Applicant: MTEX CARDIO AG, Pfaffikon (CH)

(72) Inventors: Andrea GUIDOTTI, Zurich (CH); Monica TOCCHI, Zurich (CH); Karl Heinz KUCK, Hamburg (DE); Michael BUTSCHEID, Wilen bei Wollerau (CH); Pietro GOZZOLI, Zurich (CH)

(73) Assignee: MTEX CARDIO AG, Pfaffikon (CH)

(21) Appl. No.: 18/291,060

(22) PCT Filed: Jan. 26, 2022
(86) PCT No.: PCT/IB2022/050669

§ 371 Date: Jan. 22, 2024

Related U.S. Application Data
(60) Provisional application No. 63/224,465, filed on Jul. 22, 2021.

(51) Int. Cl. A61F 2/24

(52) U.S. Cl. CPC A61F 2/2463; A61F 2/2418; A61F 2/2427; A61F 2/2466; A61F 2220/0008; A61F 2220/0025; A61F 2230/0004; A61F 2250/0003; A61F 2250/0007; A61F 2250/0018; A61F 2250/0037

(57) ABSTRACT

A cardiac assembly (1520, 1620, 1920) includes a cardiac treatment device (1532, 2132), which is configured to replace or improve function of a native atrioventricular valve, and a ventricular anchor (1530), which is configured to be positioned in a ventricle of the heart so as to support the cardiac treatment device (1532, 2132). The ventricular anchor (1530) includes (a) a proximal subannular anchor (1554), which is configured to be positioned at least partially in a target subannular space; (b) a distal anchor (1558), which is configured to be positioned partially against the ventricular wall outside the target subannular space, so as to push on the proximal subannular anchor (1554) to help anchor it in place within the target subannular space; and (c) a springy linking section (1537), which connects the proximal subannular anchor (1554) to the distal anchor (1558) such that they can articulate with respect to each other with at least one degree of freedom. Other embodiments are also described.

### CROSS-REFERENCE TO RELATED APPLICATIONS

The present application claims priority from U.S. Provisional Application 63/224,465, filed Jul. 22, 2021, which is assigned to the assignee of the present application and incorporated herein by reference.

### FIELD OF THE INVENTION

The present invention relates generally to medical devices and methods. More particularly, this invention relates to prosthetic devices and methods for improving the function of regurgitating heart valves and other circulatory valves.

### BACKGROUND OF THE APPLICATION

Valvular regurgitation (VR) is a disease in which the heart's native valve does not close properly, causing blood to flow backward into the atrium when the ventricle contracts, reducing its efficiency. Severe regurgitation affects more than 5 million patients in the U.S. today and is estimated to affect 8% of the world population.

### SUMMARY OF THE APPLICATION

Some embodiments of the present invention provide a cardiac assembly for application to a native atrioventricular valve of a heart. The cardiac assembly comprises a cardiac treatment device, which is configured to replace or improve function of the native atrioventricular valve. The cardiac assembly further comprises a ventricular anchor, which is configured to be positioned in a ventricle of the heart so as to support the cardiac treatment device.

The ventricular anchor comprises:
a proximal subannular anchor, which is configured to be positioned at least partially in a target subannular space defined by the target native leaflet and a superior portion of a ventricular wall of the ventricle; and
a distal anchor, which is configured to be positioned partially against the ventricular wall outside the target subannular space, so as to push on the proximal subannular anchor to help anchor the proximal subannular anchor in place within the target subannular space.

In some applications of the present invention, the cardiac assembly comprises a coaptation-assist device for treating the native atrioventricular valve. The cardiac treatment device comprises a neo-leaflet, which is supported by the ventricular anchor and is configured to at least partially replace function of a target native leaflet of the native atrioventricular valve by providing a surface of coaptation for one or more opposing native leaflets.

In some configurations, the ventricular anchor comprises a proximal subannular anchor, which includes a digitate anchor that is shaped so as to define a plurality of fingers having a plurality of curved superior peaks that point in a superior direction and engage a subannular surface of the target native leaflet.

There is therefore provided, in accordance with an application of the present invention, a cardiac assembly for application to a native atrioventricular valve of a heart, the cardiac assembly including:
a cardiac treatment device, which is configured to replace or improve function of the native atrioventricular valve; and
a ventricular anchor, which is configured to be positioned in a ventricle of the heart so as to support the cardiac treatment device, and which includes:
   a proximal subannular anchor, which is configured to be positioned at least partially in a target subannular space defined by the target native leaflet and a superior portion of a ventricular wall of the ventricle;
   a distal anchor, which is configured to be positioned partially against the ventricular wall outside the target subannular space, so as to push on the proximal subannular anchor to help anchor the proximal subannular anchor in place within the target subannular space; and
   a springy linking section, which connects the proximal subannular anchor to the distal anchor such that the proximal subannular anchor and the distal anchor can articulate with respect to each other with at least one degree of freedom.

For some applications, the springy linking section is axially extensible and contractable, so as to provide a variable distance between the proximal subannular anchor and the distal anchor.

For some applications, the springy linking section connects the proximal subannular anchor to the distal anchor such that the proximal subannular anchor and the distal anchor can articulate with respect to each other with at least two degrees of freedom.

For some applications, the springy linking section is more flexible about a first axis of rotation perpendicular to a longitudinal central axis of the springy linking section than about a second axis of rotation perpendicular to the longitudinal central axis of the springy linking section, the second axis perpendicular to the first axis.

For some applications, the springy linking section is serpentine and is shaped so as to define one or more undulations.

For some applications,
the ventricular anchor includes one or more wires, and the springy linking section is defined at least in part by at least one of the one or more wires,
the serpentine springy linking section is double-serpentine and is defined by first and second wire segments of the one or more wires, such that the first wire segment defines one or more first undulations of the one or more undulations and the second wire segment defines one or more second undulations of the one or more undulations, and
the first and the second wire segments run alongside and crisscross each other.

For some applications, the one or more first undulations are out of phase with the one or more second undulations.

For some applications, the springy linking section is shaped so as to define a helix.

For some applications, the ventricular anchor includes one or more wires, and the springy linking section is defined at least in part by at least one of the one or more wires.

For some applications, the distal anchor is configured to contact the ventricular wall inferior to the target subannular space.

For some applications, the ventricular anchor is configured to be atraumatic so as not to penetrate tissue of surrounding anatomy.

For some applications, the ventricular anchor is configured to penetrate tissue of surrounding anatomy.

For some applications, the cardiac assembly does not include any elements that are configured to penetrate tissue.

For some applications, the ventricular anchor and the cardiac treatment device include separate pieces that are configured to be delivered separately to the heart and to be coupled together in the heart.

For some applications, the ventricular anchor and the cardiac treatment device are coupled to each other and are configured to be delivered to the heart while coupled together.

For some applications, the ventricular anchor includes one or more wires, and the distal anchor is defined at least in part by at least one of the one or more wires shaped as a distal wire loop.

For some applications, the at least one of the one or more wires that defines the distal anchor further defines the springy linking section.

For some applications, two longitudinal portions of the at least one of the one or more wires that defines the distal anchor and the springy linking section are fixed to each other.

For some applications, the two longitudinal portions are fixed to each other by crimping.

For some applications, the two longitudinal portions are crimped together by a tube.

For some applications, the distal wire loop is configured to be positioned in the ventricle, extending to a ventricular apical area.

For some applications, the distal wire loop is configured to remain anchored in position against the ventricular wall and one or more other ventricular structures of the ventricular apical area, when the distal anchor is positioned partially against the ventricular wall outside the target subannular space.

For some applications, the one or more other ventricular structures include one or more structures selected from the group consisting of: one or more ventricular papillary muscles, and a moderator band.

For some applications, the distal wire loop is shaped so as to define three or more lobes.

For some applications, the distal wire loop is configured to remain anchored in position by force applied by the distal wire loop to surrounding anatomy.

For some applications, the distal wire loop is configured to remain anchored in position by friction between the distal wire loop and surrounding anatomy.

For some applications, the distal anchor includes a covering that covers at least a portion of the distal wire loop.

For some applications, the covering includes a coating that coats the at least a portion of the distal wire loop.

For some applications, the covering includes a sheet of material that covers the at least a portion of the distal wire loop.

For some applications, the covering entirely covers the distal wire loop.

For some applications, the covering partially covers the distal wire loop.

For some applications, the distal wire loop is shaped so as to define two or more lobes, and the anchor covering does not cover any portion of the lobes.

For some applications, the distal wire loop is shaped so as to define two or more lobes, and the anchor covering covers only respective portions of the lobes.

For some applications, the distal wire loop is shaped so as to define two or more lobes, and the anchor covering entirely covers the lobes.

For some applications,
the one or more wires that are shaped as the distal wire loop are one or more primary wires, and
the covering includes one or more secondary wires that are coiled around the one or more primary wires.

For some applications,
the one or more wires that are shaped as the distal wire loop are one or more primary wires, and
the covering includes brush bristles that extend radially outward from the one or more primary wires.

For some applications,
the one or more wires that are shaped as the distal wire loop are one or more primary wires, and
the covering includes a spiral wire brush that is spiraled around the one or more primary wires.

For some applications, the ventricular anchor includes one or more wires, and a continuous portion of at least one of the one or more wires is shaped so as to define both the distal anchor and the springy linking section.

For some applications, the continuous portion of the at least one of the one or more wires is shaped so as to define the distal anchor, the springy linking section, and at least a portion of the proximal subannular anchor.

For some applications, the continuous portion of the at least one of the one or more wires that defines the distal anchor is shaped as a distal wire loop.

For some applications, the proximal subannular anchor is configured such that, when the proximal subannular anchor is positioned at least partially in the target subannular space, the proximal subannular anchor applies a force to a ventricular surface of the target native leaflet, so as to help anchor the ventricular anchor to the target native leaflet.

For some applications,
the cardiac assembly further includes an atrial-surface support, which is configured to be disposed below or at a level of an annulus of the native atrioventricular valve and against an atrial surface of the target native leaflet when the proximal subannular anchor is positioned at least partially in the target subannular space,
the atrial-surface support and the proximal subannular anchor are configured to grasp and sandwich at least a portion of the target native leaflet, in order to support the cardiac treatment device, and to orient the cardiac treatment device with respect to the native atrioventricular valve.

For some applications, the atrial-surface support is configured to be disposed below the annulus of the native atrioventricular valve.

For some applications, the atrial-surface support includes a frame and an atrial-surface cover, which is coupled to the frame.

For some applications,
the cardiac assembly is shaped so as to define a junction between the proximal subannular anchor and the atrial-surface support, and
the cardiac assembly is configured such that the proximal subannular anchor swings outward away from the atrial-surface support upon application of a distally-directed force to the junction while the atrial-surface support is held axially stationary.

For some applications, the native atrioventricular valve is a tricuspid valve, and the cardiac treatment device is configured to replace or improve function of the tricuspid valve, when anchored in place by the ventricular anchor.

For some applications, the target native leaflet is a native septal leaflet of the tricuspid valve, and the ventricular wall is a ventricular septal wall.

For some applications, the native atrioventricular valve is a mitral valve, and the cardiac treatment device is configured to replace or improve function of the mitral valve, when anchored in place by the ventricular anchor.

For some applications, the target native leaflet is a native anterior leaflet of the mitral valve, and the ventricular wall is a ventricular septal wall.

For some applications, the cardiac treatment device includes a neo-leaflet, which is supported by the ventricular anchor and is configured to at least partially replace function of a target native leaflet of the native atrioventricular valve by providing a surface of coaptation for one or more opposing native leaflets that oppose the target native leaflet, when anchored in place by the ventricular anchor.

For some applications, the neo-leaflet is configured such that the coaptation surface is generally static throughout a cardiac cycle of the heart upon implantation of the cardiac assembly in the heart.

For some applications, the neo-leaflet is configured such that the coaptation surface moves toward and away from the one or more opposing native leaflets during a cardiac cycle of the heart upon implantation of the cardiac assembly in the heart.

For some applications, the coaptation surface has an area of between 2 and 20 cm2.

For some applications, the cardiac assembly is configured such that the coaptation surface of the neo-leaflet crosses from an atrial side to a ventricular side of a native valvular plane, when anchored in place by the ventricular anchor.

For some applications, the neo-leaflet includes:
a neo-leaflet wire loop that defines at least a portion of a border of the neo-leaflet; and
a neo-leaflet cover, which is attached to the neo-leaflet wire loop and provides the surface of coaptation.

For some applications,
the neo-leaflet has a central longitudinal axis that is oriented in a proximal-distal direction, and
the neo-leaflet wire loop is shaped so as to define two lateral sides of the neo-leaflet that face away from the central longitudinal axis and are shaped so as to define respective curved portions having convex sides that face toward the central longitudinal axis.

For some applications, the two lateral sides are not shaped so as to further define any curved portions farther from a junction between the neo-leaflet and the ventricular anchor than the respective curved portions are from the junction.

For some applications, the respective curved portions are first respective curved portions, and the two lateral sides are shaped so as to further define respective second curved portions having concave sides that face toward the central longitudinal axis, the second curved portions closer to a junction between the neo-leaflet and the ventricular anchor than the first curved portions are to the junction.

For some applications, the respective curved portions have respective radii of curvature, each of which is between 0.25 and 5 cm.

For some applications, the neo-leaflet wire loop narrows toward a junction between the neo-leaflet and the ventricular anchor.

For some applications, the cardiac treatment device includes a prosthetic heart valve, which is configured to replace the function of the native atrioventricular valve, and which includes a frame and a plurality of prosthetic leaflets that are coupled to the frame and are configured to coapt with one another during each cardiac cycle.

For some applications, the cardiac treatment device includes a spacer, which is configured to improve the function of the native atrioventricular valve by interacting with at least a portion of at least one leaflet of the native atrioventricular valve so as to at least partially restrict blood flow through the native atrioventricular valve when the native atrioventricular valve is closed during each cardiac cycle.

For some applications, the apparatus further includes at least one pouch, which is configured to inflate by blood flow during a cardiac cycle of the heart, so as to push the cardiac assembly against one or more of: a ventricular surface of the target native leaflet and an annulus of the native atrioventricular valve, thereby stabilizing the cardiac assembly with respect to the native atrioventricular valve.

For some applications, the proximal subannular anchor generally defines a curved surface having a concave side that faces toward the cardiac treatment device.

For some applications,
the ventricular anchor includes one or more wires, and
the proximal subannular anchor (a) includes a digitate anchor that (i) is defined at least in part by at least one of the one or more wires, and (ii) is shaped so as to define a plurality of fingers having a plurality of curved superior peaks, and (b) is configured to be positioned at least partially in the target subannular space such that (i) the curved superior peaks point in a superior direction and engage a subannular surface of the target native leaflet, and (ii) one or more of the fingers engage chordae tendineae that extend between the target native leaflet and the superior portion of the ventricular wall, so as to stabilize the cardiac assembly.

For some applications, the digitate anchor further includes a digitate cover, which is coupled to one or more of the fingers, and is digitate-shaped like the fingers.

For some applications, a system is provided that further includes a delivery tube in which the cardiac assembly is removably disposed in a compressed configuration for delivery to the heart.

There is further provided, in accordance with an application of the present invention, a cardiac assembly for application to a native atrioventricular valve of a heart, the cardiac assembly including:
a prosthetic heart valve, which is configured to replace function of the native atrioventricular valve, and which includes a frame and a plurality of prosthetic leaflets that are coupled to the frame and are configured to coapt with one another during each cardiac cycle; and
a ventricular anchor, which includes one or more wires, and which is configured to be positioned in a ventricle of the heart so as to support the prosthetic heart valve, and which includes:
   a proximal subannular anchor, which (a) includes a digitate anchor that (i) is defined at least in part by at least one of the one or more wires, (ii) is shaped so as to define a plurality of fingers having a plurality of curved superior peaks, and (b) is configured to be positioned at least partially in a target subannular space defined by the target native leaflet and a superior portion of a ventricular wall of the ventricle, such that (i) the curved superior peaks point in a superior direction and engage a subannular surface of the target native leaflet, and (ii) one or more of the fingers engage chordae tendineae that extend between the target native leaflet and the superior portion of the ventricular wall, so as to stabilize the cardiac assembly, and
   a distal anchor, which is configured to be positioned partially against the ventricular wall outside the target subannular space, so as to push on the proximal subannular anchor to help anchor the proximal subannular anchor in place within the target subannular space.

For some applications, the ventricular anchor and the prosthetic heart valve include separate pieces that are configured to be delivered separately to the heart and to be coupled together in the heart.

For some applications, the ventricular anchor and the prosthetic heart valve are coupled to each other and are configured to be delivered to the heart while coupled together.

There is still further provided, in accordance with an application of the present invention, a cardiac assembly for application to a native atrioventricular valve of a heart, the cardiac assembly including:
a spacer, which is configured to replace improve function of the native atrioventricular valve by interacting with at least a portion of at least one leaflet of the native atrioventricular valve so as to at least partially restrict blood flow through the native atrioventricular valve when the native atrioventricular valve is closed during each cardiac cycle; and
a ventricular anchor, which includes one or more wires, and which is configured to be positioned in a ventricle of the heart so as to support the spacer, and which includes:
   a proximal subannular anchor, which (a) includes a digitate anchor that (i) is defined at least in part by at least one of the one or more wires, (ii) is shaped so as to define a plurality of fingers having a plurality of curved superior peaks, and (b) is configured to be positioned at least partially in a target subannular space defined by the target native leaflet and a superior portion of a ventricular wall of the ventricle, such that (i) the curved superior peaks point in a superior direction and engage a subannular surface of the target native leaflet, and (ii) one or more of the fingers engage chordae tendineae that extend between the target native leaflet and the superior portion of the ventricular wall, so as to stabilize the cardiac assembly, and
a distal anchor, which is configured to be positioned partially against the ventricular wall outside the target subannular space, so as to push on the proximal subannular anchor to help anchor the proximal subannular anchor in place within the target subannular space.

For some applications, the ventricular anchor and the spacer include separate pieces that are configured to be delivered separately to the heart and to be coupled together in the heart.

For some applications, the ventricular anchor and the spacer are coupled to each other and are configured to be delivered to the heart while coupled together.

For some applications, the plurality of curved superior peaks includes between two and twelve curved superior peaks.

For some applications, the plurality of curved superior peaks includes between two and twelve curved superior peaks.

For some applications, the plurality of curved superior peaks includes between four and twelve curved superior peaks.

For some applications, the plurality of curved superior peaks includes between six and twelve curved superior peaks.

For some applications, the plurality of curved superior peaks includes between six and eight curved superior peaks.

For some applications, an average of respective greatest widths of the fingers, measured laterally, is between 2 and 10 mm when the fingers are unconstrained.

For some applications, a greatest width of a portion of the digitate anchor that defines the fingers is between 20 and 60 mm, when the fingers are unconstrained.

For some applications, an average of respective distances between midpoints of the curved superior peaks of adjacent pairs of the fingers is between 3 and 9 mm when the fingers are unconstrained.

For some applications, respective distances between midpoints of the curved superior peaks of adjacent pairs of the fingers vary along a width of the digitate anchor, when the fingers are unconstrained.

For some applications, all respective distances between midpoints of the curved superior peaks of adjacent pairs of the fingers equal one another, when the fingers are unconstrained.

For some applications, an average of respective heights of the fingers is between 4 and 30 mm, the respective heights measured in a superior-inferior direction, when the fingers are unconstrained.

For some applications, an average of respective radii of curvature of the curved superior peaks is between 1 and 10 mm when the fingers are unconstrained.

For some applications, for at least one of the fingers, a radius of curvature of the curved superior peak is greater than a smallest width of the finger, measured laterally when the fingers are unconstrained.

For some applications, when the fingers are unconstrained:
the fingers have respective greatest widths and respective heights, the greatest widths measured laterally, the respective heights measured in a superior-inferior direction,
the fingers have respective ratios of the heights to the greatest widths, and
an average of the respective ratios is between 2 and 5.

For some applications, the digitate anchor is serpentine, and is shaped so as to define a plurality of undulations having the plurality of curved superior peaks connected to a plurality of inferior troughs by respective segments.

For some applications, an average of respective greatest widths of the undulations, measured between adjacent pairs of the segments connected by respective curved superior peaks, is between 2 and 10 mm when the undulations are unconstrained.

For some applications, for at least one of the undulations, a radius of curvature of the curved superior peak is greater than a smallest width of the undulation, measured between adjacent pairs of the segments connected by the curved superior peaks, when the undulations are unconstrained.

For some applications, when the undulations are unconstrained:
the undulations have respective greatest widths and respective heights, the greatest widths measured between adjacent pairs of the segments connected by respective curved peaks, the respective heights measured in a superior-inferior direction,
the undulations have respective ratios of the heights to the greatest widths, and
an average of the respective ratios is between 2 and 5.

For some applications, a continuous portion of the one or more wires is shaped so as to define both the distal anchor and the proximal subannular anchor.

For some applications, the portion of the at least one of the one or more wires that defines the distal anchor is shaped as a distal wire loop.

For some applications, the proximal subannular anchor is configured such that, when the proximal subannular anchor is positioned at least partially in the target subannular space, the digitate anchor applies a force to a ventricular surface of the target native leaflet, so as to help anchor the ventricular anchor to the target native leaflet.

For some applications, the ventricular anchor further includes a springy linking section, which connects the proximal subannular anchor to the distal anchor such that the proximal subannular anchor and the distal anchor can articulate with respect to each other with at least one degree of freedom.

For some applications, the springy linking section is axially extensible and contractable, so as to provide a variable distance between the proximal subannular anchor and the distal anchor.

For some applications, the springy linking section connects the proximal subannular anchor to the distal anchor such that the proximal subannular anchor and the distal anchor can articulate with respect to each other with at least two degrees of freedom.

For some applications, a system is provided that further includes a delivery tube in which the cardiac assembly is removably disposed in a compressed configuration for delivery to the heart.

There is additionally provided, in accordance with an application of the present invention, a cardiac assembly for application to a native atrioventricular valve of a heart, the cardiac assembly including:
a cardiac treatment device, which is configured to replace or improve function of the native atrioventricular valve; and
a ventricular anchor, which includes one or more wires, and which is configured to be positioned in a ventricle of the heart so as to support the cardiac treatment device, and which includes:
   a proximal subannular anchor, which (a) includes a digitate anchor that (i) is defined at least in part by at least one of the one or more wires, (ii) is shaped so as to define a plurality of fingers having a plurality of curved superior peaks, and (iii) includes a digitate cover, which is coupled to one or more of the fingers, and is digitate-shaped like the fingers, and (b) is configured to be positioned at least partially in a target subannular space defined by the target native leaflet and a superior portion of a ventricular wall of the ventricle, such that (i) the curved superior peaks point in a superior direction and engage a subannular surface of the target native leaflet, and (ii) one or more of the fingers engage chordae tendineae that extend between the target native leaflet and the superior portion of the ventricular wall, so as to stabilize the cardiac assembly, and
   a distal anchor, which is configured to be positioned partially against the ventricular wall outside the target subannular space, so as to push on the proximal subannular anchor to help anchor the proximal subannular anchor in place within the target subannular space.

For some applications, the plurality of curved superior peaks includes between two and twelve curved superior peaks.

For some applications, the plurality of curved superior peaks includes between two and twelve curved superior peaks.

For some applications, the plurality of curved superior peaks includes between four and twelve curved superior peaks.

For some applications, the plurality of curved superior peaks includes between six and twelve curved superior peaks.

For some applications, the plurality of curved superior peaks includes between six and eight curved superior peaks.

For some applications, an average of respective greatest widths of the fingers, measured laterally, is between 2 and 10 mm when the fingers are unconstrained.

For some applications, a greatest width of a portion of the digitate anchor that defines the fingers is between 20 and 60 mm, when the fingers are unconstrained.

For some applications, an average of respective distances between midpoints of the curved superior peaks of adjacent pairs of the fingers is between 3 and 9 mm when the fingers are unconstrained.

For some applications, respective distances between midpoints of the curved superior peaks of adjacent pairs of the fingers vary along a width of the digitate anchor, when the fingers are unconstrained.

For some applications, all respective distances between midpoints of the curved superior peaks of adjacent pairs of the fingers equal one another, when the fingers are unconstrained.

For some applications, an average of respective heights of the fingers is between 4 and 30 mm, the respective heights measured in a superior-inferior direction, when the fingers are unconstrained.

For some applications, an average of respective radii of curvature of the curved superior peaks is between 1 and 10 mm when the fingers are unconstrained.

For some applications, for at least one of the fingers, a radius of curvature of the curved superior peak is greater than a smallest width of the finger, measured laterally when the fingers are unconstrained.

For some applications, when the fingers are unconstrained:
the fingers have respective greatest widths and respective heights, the greatest widths measured laterally, the respective heights measured in a superior-inferior direction,
the fingers have respective ratios of the heights to the greatest widths, and
an average of the respective ratios is between 2 and 5.

For some applications, the cardiac treatment device includes a neo-leaflet, which is supported by the ventricular anchor and is configured to at least partially replace function of a target native leaflet of the native atrioventricular valve by providing a surface of coaptation for one or more opposing native leaflets that oppose the target native leaflet, when anchored in place by the ventricular anchor.

For some applications, at least half of the fingers terminate superiorly to an inferior edge of the surface of coaptation of the neo-leaflet.

For some applications, all of the fingers terminate superiorly to the inferior edge of the surface of coaptation of the neo-leaflet.

For some applications, the cardiac treatment device includes a prosthetic heart valve, which is configured to replace the function of the native atrioventricular valve, and which includes a frame and a plurality of prosthetic leaflets that are coupled to the frame and are configured to coapt with one another during each cardiac cycle.

For some applications, the cardiac treatment device includes a spacer, which is configured to improve the function of the native atrioventricular valve by interacting with at least a portion of at least one leaflet of the native atrioventricular valve so as to at least partially restrict blood flow through the native atrioventricular valve when the native atrioventricular valve is closed during each cardiac cycle.

For some applications, the digitate anchor is serpentine, and is shaped so as to define a plurality of undulations having the plurality of curved superior peaks connected to a plurality of inferior troughs by respective segments.

For some applications, an average of respective greatest widths of the undulations, measured between adjacent pairs of the segments connected by respective curved superior peaks, is between 2 and 10 mm when the undulations are unconstrained.

For some applications, for at least one of the undulations, a radius of curvature of the curved superior peak is greater than a smallest width of the undulation, measured between adjacent pairs of the segments connected by the curved superior peaks, when the undulations are unconstrained.

For some applications, when the undulations are unconstrained:
the undulations have respective greatest widths and respective heights, the greatest widths measured between adjacent pairs of the segments connected by respective curved peaks, the respective heights measured in a superior-inferior direction,
the undulations have respective ratios of the heights to the greatest widths, and
an average of the respective ratios is between 2 and 5.

For some applications,
the cardiac treatment device includes a neo-leaflet, which is supported by the ventricular anchor and is configured to at least partially replace function of a target native leaflet of the native atrioventricular valve by providing a surface of coaptation for one or more opposing native leaflets that oppose the target native leaflet, when anchored in place by the ventricular anchor, and
at least half of the inferior troughs terminate superiorly to an inferior edge of the surface of coaptation of the neo-leaflet.

For some applications, all of the inferior troughs terminate superiorly to the inferior edge of the surface of coaptation of the neo-leaflet.

For some applications, a continuous portion of the one or more wires is shaped so as to define both the distal anchor and the proximal subannular anchor.

For some applications, the portion of the at least one of the one or more wires that defines the distal anchor is shaped as a distal wire loop.

For some applications, the proximal subannular anchor is configured such that, when the proximal subannular anchor is positioned at least partially in the target subannular space, the digitate anchor applies a force to a ventricular surface of the target native leaflet, so as to help anchor the ventricular anchor to the target native leaflet.

For some applications, a system is provided that further includes a delivery tube in which the cardiac assembly is removably disposed in a compressed configuration for delivery to the heart.

There is yet additionally provided, in accordance with an application of the present invention, a cardiac assembly for application to a native atrioventricular valve of a heart, the cardiac assembly including:
a cardiac treatment device, which is configured to replace or improve function of the native atrioventricular valve; and
a ventricular anchor, which is configured to be positioned in a ventricle of the heart so as to support the cardiac treatment device, and which includes:
   a proximal subannular anchor, which is configured to be positioned at least partially in a target subannular space defined by the target native leaflet and a superior portion of a ventricular wall of the ventricle; and
   a distal anchor, which (a) is defined at least in part by at least one of the one or more wires shaped as a distal wire loop, (b) includes a wire-loop covering that covers at least a portion of the distal wire loop, and (c) is configured to be positioned partially against the ventricular wall outside the target subannular space, so as to push on the proximal subannular anchor to help anchor the proximal subannular anchor in place within the target subannular space.

For some applications, the wire-loop covering includes a coating that coats the at least a portion of the distal wire loop.

For some applications, the wire-loop covering includes a sheet of material that covers the at least a portion of the distal wire loop.

For some applications,
the one or more wires that are shaped as the distal wire loop are one or more primary wires, and
the wire-loop covering includes a secondary wire that is coiled around the one or more primary wires.

For some applications, the ventricular anchor and the cardiac treatment device include separate pieces that are configured to be delivered separately to the heart and to be coupled together in the heart.

For some applications, the ventricular anchor and the cardiac treatment device are coupled to each other and are configured to be delivered to the heart while coupled together.

For some applications, the distal wire loop is configured to be positioned in the ventricle, extending to a ventricular apical area.

For some applications, the distal wire loop is configured to remain anchored in position against the ventricular wall and one or more other ventricular structures of the ventricular apical area, when the distal anchor is positioned partially against the ventricular wall outside the target subannular space.

For some applications, the one or more other ventricular structures include one or more structures selected from the group consisting of: one or more ventricular papillary muscles, and a moderator band.

For some applications, the distal wire loop is shaped so as to define three or more lobes.

For some applications, the distal wire loop is configured to remain anchored in position by force applied by the distal wire loop to surrounding anatomy.

For some applications, the distal wire loop is configured to remain anchored in position by friction between the distal wire loop and surrounding anatomy.

For some applications, a continuous portion of the one or more wires is shaped so as to define both the distal wire loop and at least a portion of the proximal subannular anchor.

For some applications, the cardiac treatment device includes a neo-leaflet, which is supported by the ventricular anchor and is configured to at least partially replace function of a target native leaflet of the native atrioventricular valve by providing a surface of coaptation for one or more opposing native leaflets that oppose the target native leaflet, when anchored in place by the ventricular anchor.

For some applications, the cardiac treatment device includes a prosthetic heart valve, which is configured to replace the function of the native atrioventricular valve, and which includes a frame and a plurality of prosthetic leaflets that are coupled to the frame and are configured to coapt with one another during each cardiac cycle.

For some applications, the cardiac treatment device includes a spacer, which is configured to improve the function of the native atrioventricular valve by interacting with at least a portion of at least one leaflet of the native atrioventricular valve so as to at least partially restrict blood flow through the native atrioventricular valve when the native atrioventricular valve is closed during each cardiac cycle.

There is also provided, in accordance with an application of the present invention, a cardiac assembly for application to a native atrioventricular valve of a heart, the cardiac assembly including:
a ventricular anchor, which is configured to be positioned in a ventricle of the heart, and which includes:
   (i) a proximal subannular anchor, which is configured to be positioned at least partially in a target subannular space defined by the target native leaflet and a superior portion of a ventricular wall of the ventricle; and
   (ii) a distal anchor, which is configured to be positioned partially against the ventricular wall outside the target subannular space, so as to push on the proximal subannular anchor to help anchor the proximal subannular anchor in place within the target subannular space;
a cardiac treatment device, which is configured to replace or improve function of the native atrioventricular valve;
an atrial-surface support, which is configured to be disposed below or at a level of an annulus of the native atrioventricular valve and against an atrial surface of the target native leaflet when the proximal subannular anchor is positioned at least partially in the target subannular space; and
a proximal supra-leaflet support, which is coupled to the atrial-surface support,
(a) the atrial-surface support and the proximal supra-leaflet support and (b) the proximal subannular anchor are configured to grasp and sandwich at least a portion of the target native leaflet, in order to support the cardiac treatment device, and to orient the cardiac treatment device with respect to the native atrioventricular valve.

For some applications, the atrial-surface support is configured to be disposed below the annulus of the native atrioventricular valve.

For some applications, the distal anchor is configured to contact the ventricular wall inferior to the target subannular space.

For some applications, the ventricular anchor is configured to be atraumatic so as not to penetrate tissue of surrounding anatomy.

For some applications, the ventricular anchor is configured to penetrate tissue of surrounding anatomy.

For some applications, the cardiac assembly does not include any elements that are configured to penetrate tissue.

For some applications, the ventricular anchor and the cardiac treatment device include separate pieces that are configured to be delivered separately to the heart and to be coupled together in the heart.

For some applications, the ventricular anchor and the cardiac treatment device are coupled to each other and are configured to be delivered to the heart while coupled together.

For some applications, the atrial-surface support includes a frame.

For some applications, the frame defines at least a portion of a border of the atrial-surface support, and the proximal supra-leaflet support is coupled to two lateral sides of the frame.

For some applications, the atrial-surface support includes an atrial-surface cover, which is coupled to the frame.

For some applications, the atrial-surface cover is in contact with the proximal supra-leaflet support in addition to being coupled to the frame.

For some applications, the atrial-surface cover is coupled to a surface of the frame that faces the proximal subannular anchor.

For some applications, the proximal supra-leaflet support extends more proximally than a proximal-most point of the proximal subannular anchor.

For some applications, one or more proximal-most portions of the proximal supra-leaflet support are bent toward the proximal subannular anchor.

For some applications, the one or more proximal-most portions of the proximal supra-leaflet support are bent toward the proximal subannular anchor by an angle of at least degrees with respect to more distal portions of the atrial-surface support.

For some applications, the proximal supra-leaflet support includes a frame that includes a zig-zag shaped wire.

For some applications, the zig-zag shaped wire is shaped so as to define at least two proximal peaks.

For some applications, the cardiac treatment device includes a neo-leaflet, which is supported by the ventricular anchor and is configured to at least partially replace function of a target native leaflet of the native atrioventricular valve by providing a surface of coaptation for one or more opposing native leaflets that oppose the target native leaflet, when anchored in place by the ventricular anchor.

For some applications, the neo-leaflet is configured such that the coaptation surface is generally static throughout a cardiac cycle of the heart upon implantation of the cardiac assembly in the heart.

For some applications, the neo-leaflet is configured such that the coaptation surface moves toward and away from the one or more opposing native leaflets during a cardiac cycle of the heart upon implantation of the cardiac assembly in the heart.

For some applications, the coaptation surface has an area of between 2 and 20 cm2.

For some applications,
the cardiac assembly includes at least first and second wires,
the first wire is shaped so as to define the proximal supra-leaflet support and a proximal first portion of a border of the neo-leaflet, and
the second wire is shaped so as to define a second portion of the border of the neo-leaflet.

For some applications, the first wire is thicker than the second wire.

For some applications, a cross-sectional area of the first wire equals at least 150% of a cross-sectional area of the second wire.

For some applications, the second wire is shaped so as to additionally define at least a portion of the ventricular anchor.

For some applications,
the cardiac assembly includes a third wire, which is shaped so as to define at least a portion of the ventricular anchor, and
the first wire is thicker than the third wire.

For some applications, the neo-leaflet includes a neo-leaflet cover, which is attached to the proximal first portion and the second portion of the border of the neo-leaflet and provides the surface of coaptation.

For some applications, the neo-leaflet includes:
a neo-leaflet wire loop that defines at least a portion of a border of the neo-leaflet; and
a neo-leaflet cover, which is attached to the neo-leaflet wire loop and provides the surface of coaptation.

For some applications, a width of the proximal supra-leaflet support is less than a width of the neo-leaflet wire loop.

For some applications, the width of the proximal supra-leaflet support is less than 80% of the width of the neo-leaflet wire loop.

For some applications,
the neo-leaflet has a central longitudinal axis that is oriented in a proximal-distal direction, and
the neo-leaflet wire loop is shaped so as to define two lateral sides of the neo-leaflet that face away from the central longitudinal axis and are shaped so as to define respective curved portions having convex sides that face toward the central longitudinal axis.

For some applications, the two lateral sides are not shaped so as to further define any curved portions farther from a junction between the neo-leaflet and the ventricular anchor than the respective curved portions are from the junction.

For some applications, the respective curved portions are first respective curved portions, and the two lateral sides are shaped so as to further define respective second curved portions having concave sides that face toward the central longitudinal axis, the second curved portions closer to a junction between the neo-leaflet and the ventricular anchor than the first curved portions are to the junction.

For some applications, the respective curved portions have respective radii of curvature, each of which is between 0.25 and 5 cm.

For some applications, the neo-leaflet wire loop narrows toward a junction between the neo-leaflet and the ventricular anchor.

For some applications, the cardiac assembly is configured such that the coaptation surface of the neo-leaflet crosses from an atrial side to a ventricular side of a native valvular plane, when anchored in place by the ventricular anchor.

For some applications, the cardiac treatment device includes a prosthetic heart valve, which is configured to replace the function of the native atrioventricular valve, and which includes a frame and a plurality of prosthetic leaflets that are coupled to the frame and are configured to coapt with one another during each cardiac cycle.

For some applications, the cardiac treatment device includes a spacer, which is configured to improve the function of the native atrioventricular valve by interacting with at least a portion of at least one leaflet of the native atrioventricular valve so as to at least partially restrict blood flow through the native atrioventricular valve when the native atrioventricular valve is closed during each cardiac cycle.

For some applications, the ventricular anchor includes one or more wires, and the distal anchor is defined at least in part by at least one of the one or more wires shaped as a distal wire loop.

For some applications, two longitudinal portions of the at least one of the one or more wires that defines the distal anchor are fixed to each other.

For some applications, the two longitudinal portions are fixed to each other by crimping.

For some applications, the two longitudinal portions are crimped together by a tube.

For some applications, the distal wire loop is configured to be positioned in the ventricle, extending to a ventricular apical area.

For some applications, the distal wire loop is configured to remain anchored in position against the ventricular wall and one or more other ventricular structures of the ventricular apical area, when the distal anchor is positioned partially against the ventricular wall outside the target subannular space.

For some applications, the one or more other ventricular structures include one or more structures selected from the group consisting of: one or more ventricular papillary muscles, and a moderator band.

For some applications, the distal wire loop is shaped so as to define three or more lobes.

For some applications, the distal wire loop is configured to remain anchored in position by force applied by the distal wire loop to surrounding anatomy.

For some applications, the distal wire loop is configured to remain anchored in position by friction between the distal wire loop and surrounding anatomy.

For some applications, the distal anchor includes a covering that covers at least a portion of the distal wire loop.

For some applications, the covering includes a coating that coats the at least a portion of the distal wire loop.

For some applications, the covering includes a sheet of material that covers the at least a portion of the distal wire loop.

For some applications, the covering entirely covers the distal wire loop.

For some applications, the covering partially covers the distal wire loop.

For some applications, the distal wire loop is shaped so as to define two or more lobes, and the anchor covering does not cover any portion of the lobes.

For some applications, the distal wire loop is shaped so as to define two or more lobes, and the anchor covering covers only respective portions of the lobes.

For some applications, the distal wire loop is shaped so as to define two or more lobes, and the anchor covering entirely covers the lobes.

For some applications,
the one or more wires that are shaped as the distal wire loop are one or more primary wires, and
the covering includes a secondary wire that is coiled around the one or more primary wires.

For some applications, the ventricular anchor includes one or more wires, and a continuous portion of the one or more wires is shaped so as to define both the distal anchor and at least a portion of the proximal subannular anchor.

For some applications, the portion of the at least one of the one or more wires that defines the distal anchor is shaped as a distal wire loop.

For some applications, the proximal subannular anchor is configured such that, when the proximal subannular anchor is positioned at least partially in the target subannular space, the proximal subannular anchor applies a force to a ventricular surface of the target native leaflet, so as to help anchor the ventricular anchor to the target native leaflet.

For some applications, the native atrioventricular valve is a tricuspid valve, and the cardiac treatment device is configured to replace or improve function of the tricuspid valve, when anchored in place by the ventricular anchor.

For some applications, the target native leaflet is a native septal leaflet of the tricuspid valve, and the ventricular wall is a ventricular septal wall.

For some applications, the native atrioventricular valve is a mitral valve, and the cardiac treatment device is configured to replace or improve function of the mitral valve, when anchored in place by the ventricular anchor.

For some applications, the target native leaflet is a native anterior leaflet of the mitral valve, and the ventricular wall is a ventricular septal wall.

For some applications, the apparatus further includes at least one pouch, which is configured to inflate by blood flow during a cardiac cycle of the heart, so as to push the cardiac assembly against one or more of: a ventricular surface of the target native leaflet and an annulus of the native atrioventricular valve, thereby stabilizing the cardiac assembly with respect to the native atrioventricular valve.

For some applications, the proximal subannular anchor generally defines a curved surface having a concave side that faces toward the cardiac treatment device.

For some applications,
the ventricular anchor includes one or more wires, and
the proximal subannular anchor (a) includes a digitate anchor that (i) is defined at least in part by at least one of the one or more wires, and (ii) is shaped so as to define a plurality of fingers having a plurality of curved superior peaks, and (b) is configured to be positioned at least partially in the target subannular space such that (i) the curved superior peaks point in a superior direction and engage a subannular surface of the target native leaflet, and (ii) one or more of the fingers engage chordae tendineae that extend between the target native leaflet and the superior portion of the ventricular wall, so as to stabilize the cardiac assembly.

For some applications, the digitate anchor further includes a digitate cover, which is coupled to one or more of the fingers, and is digitate-shaped like the fingers.

For some applications,
the cardiac assembly is shaped so as to define a junction between the proximal subannular anchor and the atrial-surface support, and
the cardiac assembly is configured such that the proximal subannular anchor swings outward away from the atrial-surface support upon application of a distally-directed force to the junction while the atrial-surface support is held axially stationary.

For some applications, a system is provided that further includes a delivery tube in which the cardiac assembly is removably disposed in a compressed configuration for delivery to the heart.

There is further provided, in accordance with an application of the present invention, a cardiac assembly for application to a native atrioventricular valve of a heart, the cardiac assembly including:
a ventricular anchor, which is configured to be positioned in a ventricle of the heart, and which includes:
   (i) a proximal subannular anchor, which is configured to be positioned at least partially in a target subannular space defined by the target native leaflet and a superior portion of a ventricular wall of the ventricle; and
   (ii) a distal anchor, which is configured to be positioned partially against the ventricular wall outside the target subannular space, so as to push on the proximal subannular anchor to help anchor the proximal subannular anchor in place within the target subannular space;
a cardiac treatment device, which is configured to replace or improve function of the native atrioventricular valve; and
one or more proximal loops.

For some applications, the cardiac assembly includes two or more proximal loops.

For some applications, the one or more proximal loops have a greatest outer dimension of between 2 and 10 mm.

For some applications, the one or more proximal loops are elliptical or circular.

For some applications, each of the one or more proximal loops is shaped so as to define at least two turns.

For some applications, the distal anchor is configured to contact the ventricular wall inferior to the target subannular space.

For some applications, the ventricular anchor is configured to be atraumatic so as not to penetrate tissue of surrounding anatomy.

For some applications, the ventricular anchor is configured to penetrate tissue of surrounding anatomy.

For some applications, the cardiac assembly does not include any elements that are configured to penetrate tissue.

For some applications, the ventricular anchor and the cardiac treatment device include separate pieces that are configured to be delivered separately to the heart and to be coupled together in the heart.

For some applications, the ventricular anchor and the cardiac treatment device are coupled to each other and are configured to be delivered to the heart while coupled together.

For some applications, the cardiac treatment device includes a neo-leaflet, which is supported by the ventricular anchor and is configured to at least partially replace function of a target native leaflet of the native atrioventricular valve by providing a surface of coaptation for one or more opposing native leaflets that oppose the target native leaflet, when anchored in place by the ventricular anchor.

For some applications, the one or more proximal loops are located at a junction between the neo-leaflet and the ventricular anchor.

For some applications, the neo-leaflet is configured such that the coaptation surface is generally static throughout a cardiac cycle of the heart upon implantation of the cardiac assembly in the heart.

For some applications, the neo-leaflet is configured such that the coaptation surface moves toward and away from the one or more opposing native leaflets during a cardiac cycle of the heart upon implantation of the cardiac assembly in the heart.

For some applications, the coaptation surface has an area of between 2 and 20 cm2.

For some applications, the cardiac assembly includes a wire that is shaped so as to define the one or more proximal loops, at least a portion of the ventricular anchor, and at least a portion of a border of the neo-leaflet.

For some applications, the cardiac assembly is configured such that the coaptation surface of the neo-leaflet crosses from an atrial side to a ventricular side of a native valvular plane, when anchored in place by the ventricular anchor.

For some applications, the one or more proximal loops are located at a junction between the neo-leaflet and the ventricular anchor.

For some applications, the neo-leaflet includes:
a neo-leaflet wire loop that defines at least a portion of a border of the neo-leaflet; and
a neo-leaflet cover, which is attached to the neo-leaflet wire loop and provides the surface of coaptation.

For some applications,
the neo-leaflet has a central longitudinal axis that is oriented in a proximal-distal direction, and
the neo-leaflet wire loop is shaped so as to define two lateral sides of the neo-leaflet that face away from the central longitudinal axis and are shaped so as to define respective curved portions having convex sides that face toward the central longitudinal axis.

For some applications, the two lateral sides are not shaped so as to further define any curved portions farther from a junction between the neo-leaflet and the ventricular anchor than the respective curved portions are from the junction.

For some applications, the respective curved portions are first respective curved portions, and the two lateral sides are shaped so as to further define respective second curved portions having concave sides that face toward the central longitudinal axis, the second curved portions closer to a junction between the neo-leaflet and the ventricular anchor than the first curved portions are to the junction.

For some applications, the respective curved portions have respective radii of curvature, each of which is between 0.25 and 5 cm.

For some applications, the neo-leaflet wire loop narrows toward a junction between the neo-leaflet and the ventricular anchor.

For some applications, the cardiac treatment device includes a prosthetic heart valve, which is configured to replace the function of the native atrioventricular valve, and which includes a frame and a plurality of prosthetic leaflets that are coupled to the frame and are configured to coapt with one another during each cardiac cycle.

For some applications, the cardiac treatment device includes a spacer, which is configured to improve the function of the native atrioventricular valve by interacting with at least a portion of at least one leaflet of the native atrioventricular valve so as to at least partially restrict blood flow through the native atrioventricular valve when the native atrioventricular valve is closed during each cardiac cycle.

For some applications, the ventricular anchor includes one or more wires, and the distal anchor is defined at least in part by at least one of the one or more wires shaped as a distal wire loop.

For some applications, two longitudinal portions of the at least one of the one or more wires that defines the distal anchor are fixed to each other.

For some applications, the two longitudinal portions are fixed to each other by crimping.

For some applications, the two longitudinal portions are crimped together by a tube.

For some applications, the distal wire loop is configured to be positioned in the ventricle, extending to a ventricular apical area.

For some applications, the distal wire loop is configured to remain anchored in position against the ventricular wall and one or more other ventricular structures of the ventricular apical area, when the distal anchor is positioned partially against the ventricular wall outside the target subannular space.

For some applications, the one or more other ventricular structures include one or more structures selected from the group consisting of: one or more ventricular papillary muscles, and a moderator band.

For some applications, the distal wire loop is shaped so as to define three or more lobes.

For some applications, the distal wire loop is configured to remain anchored in position by force applied by the distal wire loop to surrounding anatomy.

For some applications, the distal wire loop is configured to remain anchored in position by friction between the distal wire loop and surrounding anatomy.

For some applications, the distal anchor includes a covering that covers at least a portion of the distal wire loop.

For some applications, the covering includes a coating that coats the at least a portion of the distal wire loop.

For some applications, the covering includes a sheet of material that covers the at least a portion of the distal wire loop.

For some applications, the covering entirely covers the distal wire loop.

For some applications, the covering partially covers the distal wire loop.

For some applications, the distal wire loop is shaped so as to define two or more lobes, and the anchor covering does not cover any portion of the lobes.

For some applications, the distal wire loop is shaped so as to define two or more lobes, and the anchor covering covers only respective portions of the lobes.

For some applications, the distal wire loop is shaped so as to define two or more lobes, and the anchor covering entirely covers the lobes.

For some applications,
the one or more wires that are shaped as the distal wire loop are one or more primary wires, and
the covering includes a secondary wire that is coiled around the one or more primary wires.

For some applications, the ventricular anchor includes one or more wires, and a continuous portion of the one or more wires is shaped so as to define both the distal anchor and at least a portion of the proximal subannular anchor.

For some applications, the portion of the at least one of the one or more wires that defines the distal anchor is shaped as a distal wire loop.

For some applications, the proximal subannular anchor is configured such that, when the proximal subannular anchor is positioned at least partially in the target subannular space, the proximal subannular anchor applies a force to a ventricular surface of the target native leaflet, so as to help anchor the ventricular anchor to the target native leaflet.

For some applications, the native atrioventricular valve is a tricuspid valve, and the cardiac treatment device is configured to replace or improve function of the tricuspid valve, when anchored in place by the ventricular anchor.

For some applications, the target native leaflet is a native septal leaflet of the tricuspid valve, and the ventricular wall is a ventricular septal wall.

For some applications, the native atrioventricular valve is a mitral valve, and the cardiac treatment device is configured to replace or improve function of the mitral valve.

For some applications, the target native leaflet is a native anterior leaflet of the mitral valve, and the ventricular wall is a ventricular septal wall.

For some applications, the apparatus further includes at least one pouch, which is configured to inflate by blood flow during a cardiac cycle of the heart, so as to push the cardiac assembly against one or more of: a ventricular surface of the target native leaflet and an annulus of the native atrioventricular valve, thereby stabilizing the cardiac assembly with respect to the native atrioventricular valve.

For some applications, the proximal subannular anchor generally defines a curved surface having a concave side that faces toward the cardiac treatment device.

For some applications,
the ventricular anchor includes one or more wires, and
the proximal subannular anchor (a) includes a digitate anchor that (i) is defined at least in part by at least one of the one or more wires, and (ii) is shaped so as to define a plurality of fingers having a plurality of curved superior peaks, and (b) is configured to be positioned at least partially in the target subannular space such that (i) the curved superior peaks point in a superior direction and engage a subannular surface of the target native leaflet, and (ii) one or more of the fingers engage chordae tendineae that extend between the target native leaflet and the superior portion of the ventricular wall, so as to stabilize the cardiac assembly.

For some applications, the digitate anchor further includes a digitate cover, which is coupled to one or more of the fingers, and is digitate-shaped like the fingers.

For some applications, the apparatus further includes an atrial-surface support, which is configured to be disposed below or at a level of an annulus of the native atrioventricular valve and against an atrial surface of the target native leaflet when the proximal subannular anchor is positioned at least partially in the target subannular space.

For some applications,
the cardiac assembly is shaped so as to define a junction between the proximal subannular anchor and the atrial-surface support, and
the cardiac assembly is configured such that the proximal subannular anchor swings outward away from the atrial-surface support upon application of a distally-directed force to the junction while the atrial-surface support is held axially stationary.

For some applications, the cardiac assembly is configured such that the proximal subannular anchor swings outward away from the atrial-surface support upon application of the distally-directed force to the junction while the atrial-surface support is held axially stationary by holding at least one of the one or more proximal loops stationary.

For some applications, a system is provided that further includes:
a delivery tube in which the cardiac assembly is removably disposed in a compressed configuration for delivery to the heart; and
one or more delivery elongate members, which are removably coupled to the one or more proximal loops, respectively.

For some applications, the apparatus further includes one or more couplers, the one or more elongate delivery members are removably coupled to the one or more proximal loops, respectively, via the one or more couplers, respectively.

For some applications,
the system is configured to deploy the cardiac assembly out of a distal end opening of the delivery tube, and
the cardiac assembly is removably disposed in the delivery tube in the compressed configuration, with the cardiac treatment device and the ventricular anchor extending distally from the one or more proximal loops.

There is still further provided, in accordance with an application of the present invention, a method including:
advancing, to within a heart, a ventricular anchor and a cardiac treatment device of a cardiac assembly, the ventricular anchor includes (a) a proximal subannular anchor, (b) a distal anchor, and (c) a springy linking section, which connects the proximal subannular anchor to the distal anchor such that the proximal subannular anchor and the distal anchor can articulate with respect to each other with at least one degree of freedom;
positioning the ventricular anchor in a ventricle of the heart such that:
   the proximal subannular anchor is positioned at least partially in a target subannular space defined by a target native leaflet of a native atrioventricular valve and a superior portion of a ventricular wall of the ventricle, and
   the distal anchor is positioned partially against the ventricular wall outside the target subannular space, so as to push on the proximal subannular anchor to help anchor the proximal subannular anchor in place within the target subannular space; and
positioning the cardiac treatment device such that the cardiac treatment device replaces or improves function of the native atrioventricular valve, when supported by the ventricular anchor.

For some applications, the cardiac treatment device includes a neo-leaflet, and positioning the cardiac treatment device includes positioning the neo-leaflet such that the neo-leaflet at least partially replaces function of the target native leaflet by providing a surface of coaptation for one or more opposing native leaflets that oppose the target native leaflet, when supported and anchored in place by the ventricular anchor.

For some applications, the cardiac treatment device includes a prosthetic heart valve, which is configured to replace the function of the native atrioventricular valve, and which includes a frame and a plurality of prosthetic leaflets that are coupled to the frame and are configured to coapt with one another during each cardiac cycle.

For some applications, the cardiac treatment device includes a spacer, which is configured to improve the function of the native atrioventricular valve by interacting with at least a portion of at least one leaflet of the native atrioventricular valve so as to at least partially restrict blood flow through the native atrioventricular valve when the native atrioventricular valve is closed during each cardiac cycle.

For some applications, the ventricular anchor and the cardiac treatment device include separate pieces, advancing the ventricular anchor and the cardiac treatment device to the heart includes separately advancing the ventricular anchor and the cardiac treatment device to the heart, and the method further includes coupling the ventricular anchor and the cardiac treatment device together in the heart.

For some applications, the ventricular anchor and the cardiac treatment device are coupled to each other, and advancing the ventricular anchor and the cardiac treatment device to the heart includes advancing the ventricular anchor and the cardiac treatment device to the heart while coupled together.

There is additionally provided, in accordance with an application of the present invention, a method for treating a native atrioventricular valve of a heart, the method including:
advancing, to within a heart, a ventricular anchor and a cardiac treatment device of a cardiac assembly, wherein the ventricular anchor includes (a) one or more wires, (b) a proximal subannular anchor, and (c) a distal anchor, which (i) is defined at least in part by at least one of the one or more wires shaped as a distal wire loop, and (ii) includes a wire-loop covering that covers at least a portion of the distal wire loop;
positioning the ventricular anchor in a ventricle of the heart such that:
   the proximal subannular anchor is positioned at least partially in a target subannular space defined by a target native leaflet of the native atrioventricular valve and a superior portion of a ventricular wall of the ventricle, and
   the distal anchor is positioned partially against the ventricular wall outside the target subannular space, so as to push on the proximal subannular anchor to help anchor the proximal subannular anchor in place within the target subannular space; and
positioning the cardiac treatment device such that the cardiac treatment device replaces or improves function of the native atrioventricular valve, when supported by the ventricular anchor.

For some applications, the cardiac treatment device includes a neo-leaflet, and positioning the cardiac treatment device includes positioning the neo-leaflet such that the neo-leaflet at least partially replaces function of the target native leaflet by providing a surface of coaptation for one or more opposing native leaflets that oppose the target native leaflet, when supported and anchored in place by the ventricular anchor.

For some applications, the cardiac treatment device includes a prosthetic heart valve, which is configured to replace the function of the native atrioventricular valve, and which includes a frame and a plurality of prosthetic leaflets that are coupled to the frame and are configured to coapt with one another during each cardiac cycle.

For some applications, the cardiac treatment device includes a spacer, which is configured to improve the function of the native atrioventricular valve by interacting with at least a portion of at least one leaflet of the native atrioventricular valve so as to at least partially restrict blood flow through the native atrioventricular valve when the native atrioventricular valve is closed during each cardiac cycle.

For some applications, the ventricular anchor and the cardiac treatment device include separate pieces, advancing the ventricular anchor and the cardiac treatment device to the heart includes separately advancing the ventricular anchor and the cardiac treatment device to the heart, and the method further includes coupling the ventricular anchor and the cardiac treatment device together in the heart.

For some applications, the ventricular anchor and the cardiac treatment device are coupled to each other, and advancing the ventricular anchor and the cardiac treatment device to the heart includes advancing the ventricular anchor and the cardiac treatment device to the heart while coupled together.

There is yet additionally provided, in accordance with an application of the present invention, a method including:
advancing, to within a heart, a ventricular anchor and a cardiac treatment device of a cardiac assembly, wherein the ventricular anchor includes a proximal subannular anchor and a distal anchor;
positioning the ventricular anchor in a ventricle of the heart such that:
   the proximal subannular anchor is positioned at least partially in a target subannular space defined by a target native leaflet of a native atrioventricular valve and a superior portion of a ventricular wall of the ventricle, and
   the distal anchor is positioned partially against the ventricular wall outside the target subannular space, so as to push on the proximal subannular anchor to help anchor the proximal subannular anchor in place within the target subannular space;
positioning the cardiac treatment device such that the cardiac treatment device replaces or improves function of the native atrioventricular valve, when supported by the ventricular anchor; and
positioning an atrial-surface support of the cardiac assembly below or at a level of an annulus of the native atrioventricular valve and against an atrial surface of the target native leaflet, such that (a) the atrial-surface support and a proximal supra-leaflet support coupled to the atrial-surface support and (b) the proximal subannular anchor grasp and sandwich at least a portion of the target native leaflet, in order to support the cardiac treatment device, and to orient the cardiac treatment device with respect to the native atrioventricular valve.

For some applications, the cardiac treatment device includes a neo-leaflet, and positioning the cardiac treatment device includes positioning the neo-leaflet such that the neo-leaflet at least partially replaces function of the target native leaflet by providing a surface of coaptation for one or more opposing native leaflets that oppose the target native leaflet, when supported and anchored in place by the ventricular anchor.

For some applications, the cardiac treatment device includes a prosthetic heart valve, which is configured to replace the function of the native atrioventricular valve, and which includes a frame and a plurality of prosthetic leaflets that are coupled to the frame and are configured to coapt with one another during each cardiac cycle.

For some applications, the cardiac treatment device includes a spacer, which is configured to improve the function of the native atrioventricular valve by interacting with at least a portion of at least one leaflet of the native atrioventricular valve so as to at least partially restrict blood flow through the native atrioventricular valve when the native atrioventricular valve is closed during each cardiac cycle.

For some applications, the ventricular anchor and the cardiac treatment device include separate pieces, advancing the ventricular anchor and the cardiac treatment device to the heart includes separately advancing the ventricular anchor and the cardiac treatment device to the heart, and the method further includes coupling the ventricular anchor and the cardiac treatment device together in the heart.

For some applications, the ventricular anchor and the cardiac treatment device are coupled to each other, and advancing the ventricular anchor and the cardiac treatment device to the heart includes advancing the ventricular anchor and the cardiac treatment device to the heart while coupled together.

There is also provided, in accordance with an application of the present invention, a method including:
advancing, to within a heart, a ventricular anchor and a cardiac treatment device of a cardiac assembly, wherein the ventricular anchor includes (a) a proximal subannular anchor, (b) a distal anchor, and (c) one or more proximal loops;
positioning the ventricular anchor in a ventricle of the heart such that:
   the proximal subannular anchor is positioned at least partially in a target subannular space defined by a target native leaflet of a native atrioventricular valve and a superior portion of a ventricular wall of the ventricle, and
   the distal anchor is positioned partially against the ventricular wall outside the target subannular space, so as to push on the proximal subannular anchor to help anchor the proximal subannular anchor in place within the target subannular space; and
positioning the cardiac treatment device such that the cardiac treatment device replaces or improves function of the native atrioventricular valve, when supported by the ventricular anchor.

For some applications, the cardiac treatment device includes a neo-leaflet, and positioning the cardiac treatment device includes positioning the neo-leaflet such that the neo-leaflet at least partially replaces function of the target native leaflet by providing a surface of coaptation for one or more opposing native leaflets that oppose the target native leaflet, when supported and anchored in place by the ventricular anchor.

For some applications, advancing the cardiac assembly includes delivering the cardiac assembly while the cardiac assembly is removably disposed in a delivery tube of a system in a compressed configuration, and while one or more delivery elongate members of the system are removably coupled to the one or more proximal loops, respectively.

For some applications, minimally-invasively or percutaneously delivering the cardiac assembly includes minimally-invasively or percutaneously delivering the cardiac assembly while one or more delivery elongate members of the system are removably coupled to the one or more proximal loops, respectively, via one or more couplers of the system, respectively.

For some applications,
minimally-invasively or percutaneously delivering the cardiac assembly includes deploying the cardiac assembly out of a distal end opening of the delivery tube, and
minimally-invasively or percutaneously delivering the cardiac assembly while the cardiac assembly is removably disposed in the delivery tube in the compressed configuration, with the cardiac treatment device and the ventricular anchor extending distally from the one or more proximal loops.

For some applications, the cardiac treatment device includes a prosthetic heart valve, which is configured to replace the function of the native atrioventricular valve, and which includes a frame and a plurality of prosthetic leaflets that are coupled to the frame and are configured to coapt with one another during each cardiac cycle.

For some applications, the cardiac treatment device includes a spacer, which is configured to improve the function of the native atrioventricular valve by interacting with at least a portion of at least one leaflet of the native atrioventricular valve so as to at least partially restrict blood flow through the native atrioventricular valve when the native atrioventricular valve is closed during each cardiac cycle.

For some applications, the ventricular anchor and the cardiac treatment device include separate pieces, advancing the ventricular anchor and the cardiac treatment device to the heart includes separately advancing the ventricular anchor and the cardiac treatment device to the heart, and the method further includes coupling the ventricular anchor and the cardiac treatment device together in the heart.

For some applications, the ventricular anchor and the cardiac treatment device are coupled to each other, and advancing the ventricular anchor and the cardiac treatment device to the heart includes advancing the ventricular anchor and the cardiac treatment device to the heart while coupled together.

The present invention will be more fully understood from the following detailed description of embodiments thereof, taken together with the drawings, in which:

### BRIEF DESCRIPTION OF THE DRAWINGS

FIGS. 1A-C are schematic illustrations of a coaptation-assist device for treating a native atrioventricular valve of a subject, in accordance with an application of the present invention;

FIG. 2 is a schematic illustration of the coaptation-assist device of FIGS. 1A-C implanted in a native valve, in accordance with an application of the present invention;

FIGS. 3A-D are schematic illustrations of another coaptation-assist device, in accordance with an application of the present invention;

FIGS. 3E-H are schematic illustrations of another coaptation-assist device, in accordance with an application of the present invention;

FIGS. 4A-D are schematic illustrations of yet another coaptation-assist device, in accordance with an application of the present invention;

FIGS. 5A-O are schematic illustrations of still another coaptation-assist device, in accordance with an application of the present invention;

FIGS. 6A-D are schematic illustrations of another coaptation-assist device, in accordance with an application of the present invention;

FIGS. 6E-F are schematic illustrations of an alternative configuration of the coaptation-assist device of FIGS. 6A-D, in accordance with an application of the present invention;

FIGS. 7A-D are schematic illustrations of an alternative configuration of the coaptation-assist device of FIGS. 5A-O, in accordance with an application of the present invention;

FIGS. 8A-C are schematic illustrations of further alternative configurations of the coaptation-assist device of FIGS. 5A-O, in accordance with respective applications of the present invention;

FIGS. 9A-D are schematic illustrations of configurations of a distal anchor, in accordance with respective applications of the present invention; and

FIG. 10 is a schematic illustration of another alternative configuration of the coaptation-assist device of FIGS. 5A-O, in accordance with an application of the present invention.

FIGS. 11A-C are schematic illustrations of a cardiac assembly in a disassembled state, in accordance with an application of the present invention;

FIG. 12 is a schematic illustration of the cardiac assembly of FIGS. 11A-C in an assembled state, in accordance with an application of the present invention;

FIG. 13 is a schematic illustration of another cardiac assembly in a disassembled state, in accordance with an application of the present invention; and

FIG. 14 is a schematic illustration of the cardiac assembly of FIG. 13 in an assembled state, in accordance with an application of the present invention.

### DETAILED DESCRIPTION OF APPLICATIONS

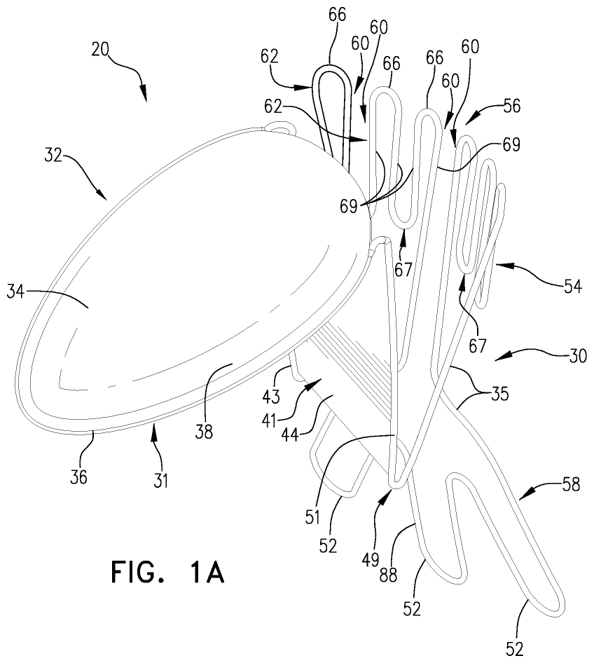

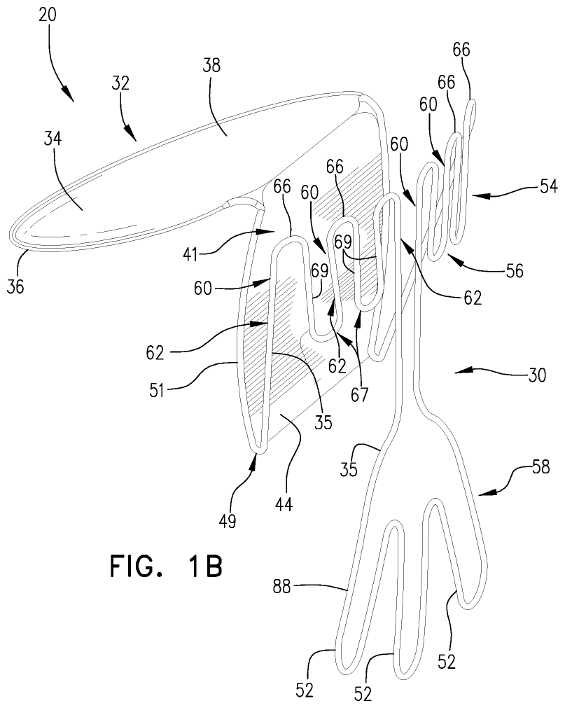

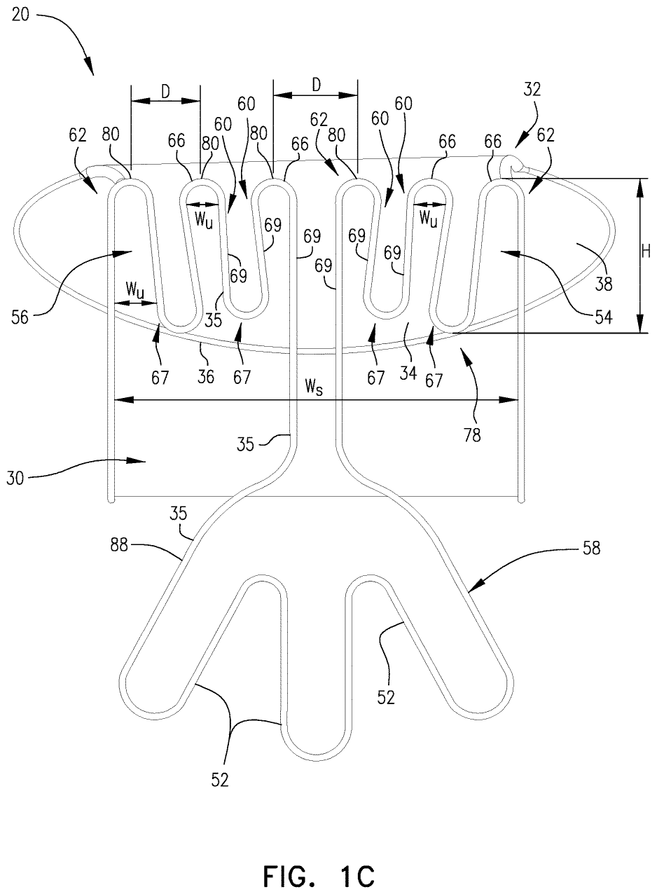

FIGS. 1A-C are schematic illustrations of a coaptation-assist device 20 for treating a native atrioventricular valve 22 of a subject, in accordance with an application of the present invention. Typically, the native atrioventricular valve suffers from a valvular pathology, such as functional mitral or tricuspid regurgitation, often characterized by lack of mobility of the leaflets in the valve. Any of the coaptation-assist devices described herein may also be referred to as cardiac assemblies.

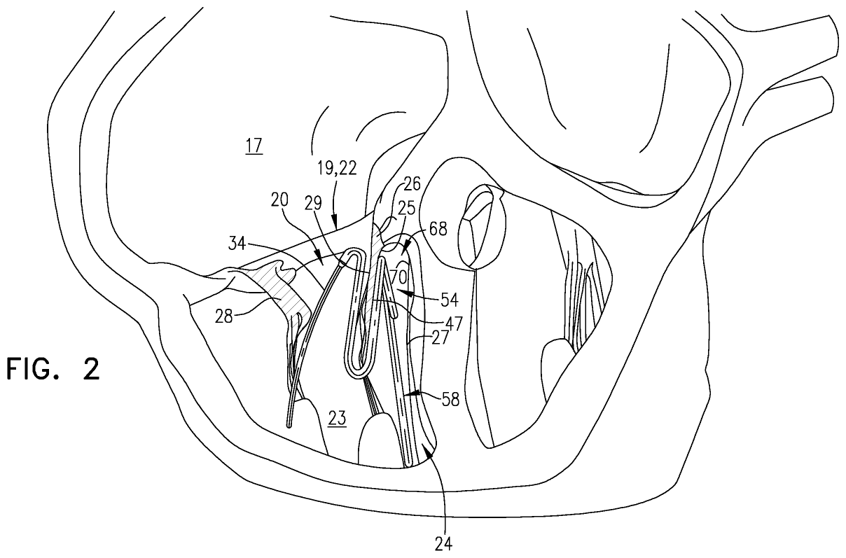

Reference is additionally made to FIG. 2, which is a schematic illustration of coaptation-assist device 20 implanted in native atrioventricular valve 22, in accordance with an application of the present invention. In the particular implantation shown in FIG. 2, native atrioventricular valve 22 is a tricuspid valve 19. FIG. 2 is a cross-sectional view of the heart, with an anterior portion of the heart, including the native anterior leaflet of tricuspid valve 19, removed, such that only the septal and posterior leaflets are shown.

Coaptation-assist device 20 comprises a ventricular anchor 30 and a neo-leaflet 32. Ventricular anchor 30 is configured to be positioned in a ventricle 23 of the heart. Neo-leaflet 32 is supported by ventricular anchor 30. As used in the present application, including in the claims and Inventive concepts, the term “neo-leaflet” means “prosthetic leaflet.”

For some applications, coaptation-assist device 20 does not comprise any elements that are configured to penetrate (e.g., pierce) tissue. For other applications, coaptation-assist device 20 comprises at least one element that is configured to penetrate tissue (configuration not shown).

Typically, ventricular anchor 30 is configured to be atraumatic so as not to penetrate (e.g., pierce) tissue of the surrounding anatomy. To this end, ventricular anchor 30 typically does not comprise any exposed sharp elements that might penetrate tissue.

Neo-leaflet 32 is configured to at least partially replace function of a target native leaflet 26 of native atrioventricular valve 22 by providing a surface of coaptation 34 for one or more opposing native leaflets 28 that oppose target native leaflet 26, when anchored in place by ventricular anchor 30, such as shown in FIG. 2. Neo-leaflet 32 is typically configured to cover at least a portion of target native leaflet 26.

For some applications, coaptation surface 34 has an area of at least 2 cm2 (e.g., at least 10 cm2 ), no more than 20 cm2 (e.g., no more than 15 cm2 ), and/or between 2 cm (e.g., 10 cm2 ) and 20 cm2 (e.g., 15 cm2 ). The other coaptation surfaces described hereinbelow may also have these areas.

For some applications, neo-leaflet 32 comprises a neo-leaflet wire loop 36 that defines at least a portion of a border 31 of the neo-leaflet (by running along or near border 31, e.g., within 2 mm of border 31). Neo-leaflet 32 further comprises a neo-leaflet cover 38 attached to neo-leaflet wire loop 36. Typically, neo-leaflet cover 38 provides the above-mentioned coaptation surface 34. For some applications, neo-leaflet cover 38 comprises one or more biocompatible thin sheets of material, which may comprise a synthetic material or a biological tissue material, such as, for example, a fabric comprising of a polymer or biomaterial (e.g., polyethylene terephthalate (PET), expanded polytetrafluoroethylene (ePTFE), silicone, urethane, or pericardium). (For example, the use of pericardium may help avoid erosion of the opposing native leaflets, e.g., the anterior and posterior leaflets of the tricuspid valve.) Neo-leaflet wire loop 36 may be shaped as a closed loop or an open loop, which is open on a proximal side in the direction of distal anchor 58 (e.g., distal wire loop 88), described hereinbelow. Neo-leaflet wire loop 36 comprises metal or another semi-rigid material. The material of neo-leaflet wire loop 36 is either self-expanding or mechanically expandable. For example, neo-leaflet wire loop 36 may comprise a shape-memory alloy, such as Nitinol. For some applications, neo-leaflet wire loop 36 is fabricated by shaping one or more wires. For other applications, neo-leaflet wire loop 36 is fabricated by wire coiling featuring fixed or variable characteristics along the loop length, including one or more of outside diameter, pitch spacing, and stiffness. For some applications, the wire of neo-leaflet wire loop 36 is circular in cross-section; alternatively, the wire has another cross-sectional shape, such as elliptical, rectangular, or generally flat. For still other applications, neo-leaflet wire loop 36 is fabricated by laser-cutting and shaping a tube or a flat shape sheet of metal, such as a shape memory alloy, e.g., Nitinol.

For some applications, neo-leaflet 32 has an intrinsic stiffness provided by the material of neo-leaflet wire loop 36 and/or neo-leaflet cover 38. Alternatively or additionally, neo-leaflet 32 comprises one or more stiffening elements within neo-leaflet cover 38 of the neo-leaflet to prevent the neo-leaflet from prolapsing in the atrial chamber as a result of an increase of backflow pressure over the neo-leaflet's ventricular surface during the cardiac cycle.

As mentioned above, for some applications, such as shown in FIG. 2, native atrioventricular valve 22 is tricuspid valve 19. For these applications, neo-leaflet 32 is configured to at least partially replace function of target native leaflet 26 by providing coaptation surface 34 for one or more opposing native leaflets 28 of tricuspid valve 19, when anchored in place by ventricular anchor 30. For some of these applications, target native leaflet 26 is a native septal leaflet of tricuspid valve 19, ventricular wall 27 (described hereinbelow) is a ventricular septal wall, and neo-leaflet 32 is configured to at least partially replace function of the septal leaflet by providing coaptation surface 34 for one or more of the opposing native posterior and anterior leaflets of tricuspid valve 19, when anchored in place by ventricular anchor 30. Although in the cross-sectional view of the heart in FIG. 2, the only opposing native leaflet 28 that is shown is the posterior native leaflet, the opposing anterior native leaflet also coapts with coaptation surface 34 when it partially replaces the septal leaflet.

For other applications (configuration not shown), native atrioventricular valve 22 is a mitral valve, and neo-leaflet 32 is configured to at least partially replace function of target native leaflet 26 (either a native posterior leaflet or a native anterior leaflet) by providing a coaptation surface 34 for the opposing native leaflet of the mitral valve, when anchored in place by ventricular anchor 30. For some of these applications, target native leaflet 26 is the native anterior leaflet of the mitral valve, ventricular wall 27 (described hereinbelow) is a ventricular septal wall, and neo-leaflet 32 is configured to at least partially replace function of the native anterior leaflet by providing coaptation surface 34 for the opposing native posterior leaflet of the mitral valve, when anchored in place by ventricular anchor 30.

Reference is still made to FIGS. 1A-C and 2. Ventricular anchor 30 comprises one or more wires 35. The one or more wires 35 comprise metal or another semi-rigid material. The wires may be conventional wires, or may be fabricated by laser-cutting and shaping a tube or a flat shape sheet of metal. The material of the one or more wires 35 is either self-expanding or mechanically expandable. For example, the one or more wires 35 may comprise a shape-memory alloy, such as Nitinol. For some applications, the one or more wires 35 are coated.

Ventricular anchor 30 comprises a proximal subannular anchor 54 and, typically, a distal anchor 58. Alternatively, for some applications, ventricular anchor 30 does not comprise distal anchor 58, and is instead anchored in place only by sandwiching at least a portion of atrial and ventricular surfaces of target native leaflet 26, such as described hereinbelow with reference to FIGS. 1A-C and 2.

Proximal subannular anchor 54 comprises a digitate anchor 56 that is defined at least in part by at least one of the one or more wires 35. Digitate anchor 56 is shaped so as to define a plurality of fingers 60 having a plurality of curved superior peaks 66. As used in the present application, including in the claims, a “finger” is an elongate, narrow projection, and “digitate” means having digits or fingerlike projections.

Alternatively, proximal subannular anchor 54 does not comprise digitate anchor 56.

For some applications, such as shown, digitate anchor 56 is serpentine, and is shaped so as to define a plurality of undulations 62 having the plurality of curved superior peaks 66 connected to a plurality of inferior troughs 67 by respective segments 69, which are generally aligned in a superior-anterior and/or diagonal direction. Inferior troughs 67 are typically curved. Undulations 62 define fingers 60, respectively. Alternatively, for some applications, digitate anchor 56 is not serpentine, i.e., does not define troughs; for example, inferior ends of fingers 60 may be connected to a horizontal (i.e., laterally-oriented) portion of wire 35 of digitate anchor 56 (configuration not shown).

For some applications, fingers 60 are covered partially or entirely with one or more biocompatible thin sheets of material. Typically, the one or more biocompatible thin sheets of material are soft and atraumatic. The one or more biocompatible thin sheets of material may comprise a synthetic material or a biological tissue material, such as, for example, a fabric comprising of a polymer or biomaterial (e.g., polyethylene terephthalate (PET), expanded polytetrafluoroethylene (ePTFE), silicone, urethane, or pericardium), or an electospun material.

Proximal subannular anchor 54 is configured to be positioned at least partially in a target subannular space 68 defined by target native leaflet 26 and a superior portion 70 of a ventricular wall 27. In other words, proximal subannular anchor 54 is configured to be secured underneath and behind target native leaflet 26, in contact with a subannular surface 25 of target native leaflet 26.

In particular, proximal subannular anchor 54 is configured to be positioned at least partially in target subannular space 68 such that:
curved superior peaks 66 point in a superior direction and engage a subannular surface 25 of target native leaflet 26; and
one or more of fingers 60 engage chordae tendineae 76 that extend between target native leaflet 26 and superior portion 70 of ventricular wall 27, so as to stabilize coaptation-assist device 20, including, in particular, neo-leaflet 32.

Fingers 60 are sufficiently narrow such that they pass around and between chordae tendineae 76 during advancement of digitate anchor 56 from the ventricular area in a superior direction into and within target subannular space 68. Optionally, fingers 60 are also sufficiently flexible such that they reposition themselves as necessary around and between chordae tendineae 76 during advancement of digitate anchor 56 in the superior direction into and within target subannular space 68.

To this end, typically:
digitate anchor 56 is shaped so as to define fingers 60 having at least two, no more than twenty, and/or between two and twenty curved superior peaks 66, such as between two and twelve curved superior peaks, e.g., between four and twelve curved superior peaks, such as between six and twelve curved superior peaks, e.g., between six and eight curved superior peaks, and/or
an average of respective greatest widths W_{U} of fingers 60, measured laterally, is at least 2 mm, no more than 10 mm, and/or between 2 and 10 mm when fingers 60 are unconstrained, such as at least 3 mm, no more than 6 mm, and/or between 3 and 6 mm, as labeled in FIG. 1C.

For some applications in which digitate anchor 56 is serpentine, an average of respective greatest widths W_{U} of undulations 62, measured between adjacent pairs of segments 69 connected by respective curved superior peaks 66, is at least 2 mm, no more than 10 mm, and/or between 2 and 10 mm when undulations 62 are unconstrained, such as at least 3 mm, no more than 6 mm, and/or between 3 and 6 mm, as labeled in FIG. 1C.

Typically, a portion of chordae tendineae 76 extend between a ventricular surface of target native leaflet 26 and superior portion 70 of ventricular wall 27 (known in the art as secondary and tertiary chordae tendineae), and a portion of chordae tendineae 76 extend between a free edge 84 of target native leaflet 26 and superior portion 70 of ventricular wall 27 (known in the art as primary chordae tendineae).

For some applications:
a greatest width W_{S} of a portion 78 of digitate anchor 56 that defines fingers 60 is between 20 and 60 mm, such as between 25 and 40 mm, when fingers 60 are unconstrained,
an average of respective distances D between midpoints 80 of curved superior peaks 66 of adjacent pairs of fingers 60 is at least 3 mm, no more than 9 mm, and/or between 3 and 9 mm when fingers 60 are unconstrained,
an average of respective heights H of fingers 60 is at least 4 mm, no more than 30 mm, and/or between 4 and 30 mm, such as at least 8 mm, no more than 12 mm, and/or between 8 and 12 mm, the respective heights measured in a superior-inferior direction, when fingers 60 are unconstrained, and/or
fingers 60 have respective ratios of the heights H to the greatest widths W_{U}, and an average of the respective ratios is at least 2, no more than 5, and/or between 2 and 5, when fingers 60 are unconstrained.

(As labeled in FIG. 1C, for applications in which digitate anchor 56 is serpentine, the above-mentioned respective heights H are measured between curved superior peaks 66 and adjacent curved inferior troughs 79 of fingers 60 in a superior-inferior direction, rather than a slanted direction directly between midpoints 80 of curved superior peaks 66 and midpoints 80 of curved inferior troughs 79.)

For some applications, an average of respective radii of curvature R_{C} of curved superior peaks 66 is at least 1 mm, no more than 10 mm, and/or between 1 and 10 mm, such as at least 3 mm, no more than 6 mm, and/or between 3 and 6 mm, when fingers 60 are unconstrained.

For some applications, for at least one of fingers 60 (such as two or more, or all, of fingers 60), a radius of curvature R_{C} of curved superior peak 66 is greater than a smallest width W_{M} of finger60, when fingers 60 are unconstrained. These relative dimensions may provide vertical integrity to digitate anchor 56. For some applications in which digitate anchor 56 is serpentine, for at least one of undulations 62 (such as two or more, or all, of undulations 62), radius of curvature R_{C} of curved peak 66 is greater than a smallest width W_{M} of undulation 62, when undulations 62 are unconstrained.

For some applications, respective distances D between midpoints 80 of curved superior peaks 66 of adjacent pairs of fingers 60 vary along a width of digitate anchor 56, when fingers 60 are unconstrained. For example, fingers 60 may be more concentrated in a central portion of the width of digitate anchor 56 than in a peripheral (i.e., commissural) portion of the width of digitate anchor 56, or vice versa. This concentration may accommodate different chordal distribution in the center of the native leaflet, and may provide more flexibility to the commissural sides while keeping stability in the center. For other applications, all respective distances D between midpoints 80 of curved superior peaks 66 of adjacent pairs of fingers 60 equal one another, when fingers 60 are unconstrained.

For some applications, at least half of the fingers 60 (such as all of fingers 60) terminate superiorly to an inferior edge 86 of surface of coaptation 34 of neo-leaflet 32. For some applications in which digitate anchor 56 is serpentine, at least half of inferior troughs 79 (such as all of inferior troughs 79) terminate superiorly to an inferior edge 86 of surface of coaptation 34 of neo-leaflet 32.

For some applications, foam is provided at one or more of curved superior peaks 66 and/or at one or more of inferior troughs 67, which may provide more atraumatic contact between digitate anchor 56 and the subannular apparatus.

Distal anchor 58 is configured to be positioned partially against ventricular wall 27 (such as a ventricular septal wall, as mentioned above) outside target subannular space 68, so as to push on proximal subannular anchor 54 to help anchor proximal subannular anchor 54 in place within target subannular space 68. For some applications, such as shown in FIGS. 1A-C and 2, distal anchor 58 is configured to contact ventricular wall 27 inferior to target subannular space 68.

For some applications, such as shown in FIGS. 1A-C and 2, distal anchor 58 is defined at least in part by at least one of the one or more wires 35.

For some these applications, the at least one of the one or more wires 35 that defines distal anchor 58 is shaped as a distal wire loop 88, which provides distal anchor 58 with a loop-shaped border. For some applications, distal wire loop 88 is fabricated by shaping the at least one of the one or more wires 35. For other applications, distal wire loop 88 is fabricated by wire coiling featuring fixed or variable characteristics along the loop length; the variable characteristics may include one or more of outside diameter, pitch spacing, and stiffness. For other applications, distal wire loop 88 is fabricated by laser-cutting and shaping a tube or a flat shape sheet of metal, such as a shape memory alloy, e.g., Nitinol.

For some applications, distal wire loop 88 is configured to be positioned in ventricle 23, extending to a ventricular apical area 24 (at the bottom of ventricle 23). Distal wire loop 88 is configured to remain anchored in position against surrounding anatomy, including subannular surface 25, a ventricular wall 27, and ventricular apical area 24, such as shown in FIG. 2. In other words, distal wire loop 88 is configured to be seated apically, and to be stabilized by ventricular wall 27. Typically, distal wire loop 88 is configured to pass behind or across ventricular papillary muscles 46 of ventricular apical area 24. Optionally, the surrounding anatomy against which distal wire loop 88 is anchored further includes one or more of the following: a moderator band, one or more chordae tendineae, and one or more papillary muscles on the opposite side of ventricle 23.

Typically, distal wire loop 88 is configured to remain anchored in position by force (typically radially-outwardly-directed force) applied by distal wire loop 88 to the surrounding anatomy, and/or by friction between distal wire loop 88 and the surrounding anatomy. For some applications, distal wire loop 88 comprises a self-expandable material, such as a shape-memory alloy (e.g., Nitinol) that causes distal wire loop 88 to expand radially outwardly so as to apply the force. For these applications, distal wire loop 88 typically is configured to have a shape in its resting (relaxed) state that is larger than the surrounding anatomy, such that the surrounding anatomy limits expansion of distal wire loop 88 and distal wire loop 88 applies a force to the surrounding anatomy (and vice versa). In addition, the narrowing of ventricular wall 27 in a subannular-to-apical direction compresses distal wire loop 88, creating a counter-radial force, and directing distal wire loop 88 to stabilize itself at the sub-leaflet ventricular hinge level (i.e., at the level of subannular surface 25).

For some applications, distal wire loop 88 is shaped so as to define three or more lobes 52, such as exactly three lobes 52, as shown in FIGS. 1A-C and 2. Lobes 52 may allow distal wire loop 88 to readily enter the wrinkled crevices defined by ventricular wall 27 at ventricular apical area 24. Typically, respective lengths of lobes 52 (measured in a generally superior-anterior direction) are greater than respective widths of lobes 52, e.g., greater than twice the respective widths of lobes 52.

For some applications, distal wire loop 88 has one or more of the following dimensions:
an enclosed (surrounded) area of at least 2 cm2, no more than 60 cm2, and/or between 2 and 60 cm2,
a perimeter of at least 4 cm, no more than 15 cm, and/or between 4 and 15 cm,
a length of at least 2 cm, no more than 12 cm, and/or between 2 and 12 cm, and/or
a width of at least 1 cm, no more than 12 cm, and/or between 1 and 12 cm.

Distal wire loop 88 may define a plane or a curved surface.

For some applications, when lobes 52 are unconstrained, lobes 52 are shaped so as to define respective best-fit planes, and at least two of the best-fit-planes are not coplanar with one another. This configuration may provide stability to distal wire loop 88, by making it more difficult for distal wire loop 88 to slide sideways, which might occur if lobes 52 were instead aligned along the same plane. This configuration may also accommodate the width of the ventricular apex area, to expand across that area and increase stabilization.

Reference is still made to FIGS. 1A-C and 2. For some applications, a continuous portion of the one or more wires 35 is shaped so as to define both distal anchor 58 and digitate anchor 56, and, optionally additional portions of proximal subannular anchor 54.

For some of these applications, the portion of the at least one of the one or more wires 35 that defines distal anchor 58 is shaped as a distal wire loop 88, which may have any of the characteristics of distal wire loop 88 described hereinabove.

Reference is still made to FIGS. 1A-C and 2. For some applications, proximal subannular anchor 54 is configured such that, when proximal subannular anchor 54 is positioned at least partially in target subannular space 68, digitate anchor 56 applies a force to ventricular surface 82 of target native leaflet 26, so as to help anchor ventricular anchor 30 to target native leaflet 26, and typically so as to support and/or stabilize neo-leaflet 32, and so as to orient neo-leaflet 32 with respect to native atrioventricular valve 22. For example, this additional anchoring may help prevent neo-leaflet 32 from tilting toward one of the native commissures, and/or may help orient neo-leaflet 32 at a desired angle with respect to the native valvular plane.

For some of these applications, coaptation-assist device 20 further comprises an atrial-surface support 41, which is configured to be disposed below or at a level of an annulus of native atrioventricular valve 22 and against an atrial surface 47 of target native leaflet 26 when proximal subannular anchor 54 is positioned at least partially in target subannular space 68. Atrial-surface support 41 and digitate anchor 56 are configured to grasp and sandwich at least a portion of target native leaflet 26, in order to support neo-leaflet 32, and to orient neo-leaflet 32 with respect to native atrioventricular valve 22.

For some applications, atrial-surface support 41 comprises a frame 51 and an atrial-surface cover 44, which is coupled to frame 51. Frame 51 comprises one or more wires 43. Typically, frame 51 defines at least a portion of a border of atrial-surface support 41 and/or atrial-surface cover 44.

For some applications, atrial-surface cover 44 comprises one or more biocompatible thin sheets of material (optionally, the same one or more sheets of material (e.g., the same exactly one sheet of material) define both neo-leaflet cover 38, described above, and atrial-surface cover 44; alternatively, separate sheets of material define neo-leaflet cover 38 and atrial-surface cover 44). For some applications, atrial-surface cover 44 pushes against atrial surface 47 of target native leaflet 26 to prevent blood flow between target native leaflet 26 and the coaptation-assist device. Typically, the one or more biocompatible thin sheets of material are soft and atraumatic. The one or more biocompatible thin sheets of material may comprise a synthetic material or a biological tissue material, such as, for example, a fabric comprising of a polymer or biomaterial (e.g., polyethylene terephthalate (PET), expanded polytetrafluoroethylene (ePTFE), silicone, urethane, or pericardium).

For some applications, a continuous portion of the one or more wires 35 is shaped so as to define proximal subannular anchor 54 and at least partially define atrial-surface support 41.

For some applications, neo-leaflet 32 is coupled to ventricular anchor 30 via atrial-surface support 41, such as shown in FIGS. 1A-C and 2. For some of these applications, coaptation-assist device 20 comprises a wire loop that is shaped so as to at least partially define neo-leaflet 32 and atrial-surface support 41, such as shown.

For some applications, coaptation-assist device 20 is shaped so as to define a fold 49 between atrial-surface support 41 and ventricular anchor 30. Fold 49 is configured to extend around the free edge of target native leaflet 26 when atrial-surface support 41 and digitate anchor 56 grasp and sandwich the at least a portion of target native leaflet 26.

Coaptation-assist device 20 may optionally be configured to implement any of the features of coaptation-assist device 20 described in PCT Publication WO 2020/148755 to Guidotti et al., with reference to FIGS. 1A-E and 2 thereof; this international application is incorporated herein by reference.

Reference is still made to FIGS. 1A-C and 2. For some applications, neo-leaflet 32 is configured such that coaptation surface 34 is generally static throughout a cardiac cycle of the subject upon implantation of coaptation-assist device 20 in a heart of the subject. In these applications, coaptation is provided by motion of the one or more opposing native leaflets 28 against the generally static coaptation surface 34 provided by neo-leaflet 32. For example, to achieve this general stasis, neo-leaflet wire loop 36 and/or neo-leaflet cover 38 of neo-leaflet 32 may be relatively stiff, and/or neo-leaflet 32 may comprise one or more stiffening elements within neo-leaflet cover 38, as described above.

For other applications, neo-leaflet 32 is configured such that coaptation surface 34 moves toward and away from the one or more opposing native leaflets 28 during a cardiac cycle of the subject upon implantation of coaptation-assist device 20 in a heart of the subject. In other words, neo-leaflet 32 is configured such that coaptation surface 34 is dynamic throughout the cardiac cycle. In these applications, coaptation is provided by motion of coaptation surface 34 provided by neo-leaflet 32 and the one or more opposing native leaflets 28. For example, to allow this motion of coaptation surface 34, neo-leaflet wire loop 36 and/or neo-leaflet cover 38 of neo-leaflet 32 may be flexible enough to allow it to move during the cardiac cycle. Further alternatively, the neo-leaflet cover may have a surface area that is greater than an area defined and surrounded by the neo-leaflet wire loop, so as to create a flexible parachute-like coaptation surface that inflates and relaxes during the cardiac cycle. During systole surface of coaptation 34 increases, causing the neo-leaflet to be closer to the one or more opposing native leaflets 28, so as to better prevent regurgitation. The flexible parachute-like coaptation surface is configured to inflate and relax along the blood flow and pressure variation against the ventricular surface of coaptation during the cardiac cycle, so that both the blood flow and blood pressure increase and are directed against the ventricular surface of the neo-leaflet during the systolic phase of the cardiac cycle. As a result, the neo-leaflet inflates and dynamically increases the surface of coaptation for the one or more opposing native leaflets 28. The blood flow and blood pressure action against the neo-leaflet ventricular surface decrease during the diastolic phase, causing the neo-leaflet to relax and deflate, leaving the neo-leaflet structure in a resting shape during the diastolic phase, so as not to occlude the orifice area blood passage from the atrial to the ventricular chamber. In other words, during diastole, the neo-leaflet deflates and relaxes, allowing passage of blood from the atrium to ventricle 23.

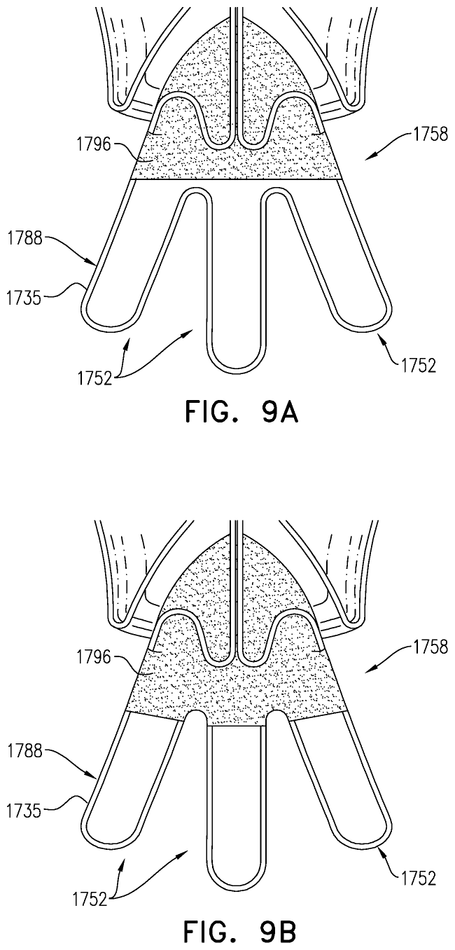

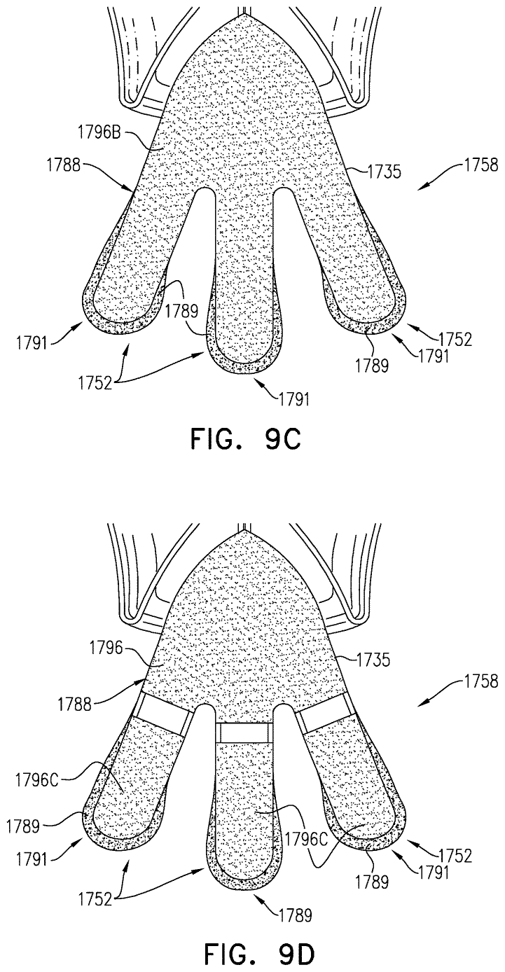

Reference is again made to FIG. 2. In an application of the present invention, a method is provided for implanting coaptation-assist device 20 in a heart of a subject. For some applications, the method is performed partially using techniques described with reference to FIGS. 9A-H of above-mentioned PCT Publication WO 2020/148755 to Guidotti et al., mutatis mutandis.

Coaptation-assist device 20 is percutaneously (endovascularly) delivered to a heart of the subject while coaptation-assist device 20 is removably disposed in a delivery tube of a delivery system in a compressed configuration. For some applications, coaptation-assist device 20 is loaded in the compressed configuration into the delivery tube by extending and flattening coaptation-assist device 20 into an elongate flattened configuration, and then further radially compressing (e.g., crimping) the device. For example, for applications in which coaptation-assist device 20 is used to treat tricuspid valve 19, such as shown in FIG. 2, the delivery tube may be advanced into a right atrium 17 via the inferior or superior vena cava. For applications in which coaptation-assist device 20 is used to treat a mitral valve, the delivery tube may be advanced transseptally into a left ventricle, using transseptal advancement techniques known in the art. Alternatively, coaptation-assist device 20 is delivered in a minimally-invasive procedure.

The delivery system typically comprises the delivery tube and optionally one or more additional tubes. One or more of the tubes is steerable (e.g., two are steerable, one for trajectory, and the other for positioning). Optionally, coaptation-assist device 20 is partially disposed in the delivery tube and partially disposed in another of the tubes, to allow sequential deployment of the elements of coaptation-assist device 20. For configurations in which coaptation-assist device 20 is entirely disposed within the delivery tube, typically distal anchor 58 is disposed more distally within the tube than neo-leaflet 32 (i.e., closer to the distal end of the tube), to allow distal anchor 58 to be deployed from the tube before neo-leaflet 32 is deployed. Alternatively, the arrangement is reversed, to allow neo-leaflet 32 to be deployed from the tube before distal anchor 58 is deployed.

Thereafter, distal anchor 58 is deployed from the delivery tube and positioned in ventricle 23. Typically, distal anchor 58 is deployed by proximally withdrawing the delivery tube and/or pushing the loop-shaped ventricular anchor from the delivery tube.

Thereafter, neo-leaflet 32 is deployed from the delivery tube and positioned such that neo-leaflet 32 at least partially replaces function of target native leaflet 26 by providing coaptation surface 34 for one or more opposing native leaflets 28 that oppose target native leaflet 26. Typically, distal anchor 58 is deployed by proximally withdrawing the delivery tube.

Although distal anchor 58 is shown and described as being deployed before neo-leaflet 32, in some configurations the order of deployment is reversed.

For some applications, ventricular anchor 30 is configured to be atraumatic, and positioning ventricular anchor 30 does not comprise penetrating tissue of the surrounding anatomy with ventricular anchor 30. For some applications, the deployment method does not comprise penetrating tissue with any elements of coaptation-assist device 20.

For some applications, as shown in FIG. 2, neo-leaflet 32 is positioned such that coaptation surface 34 of neo-leaflet 32 crosses from an atrial side to a ventricular side of a native valvular plane, when anchored in place, so that the native leaflet 28 coapts with neo-leaflet 32 during the cardiac cycle, with the atrial surface of native leaflet 28 coming into contact with an atrial surface 29 of neo-leaflet 32, thereby stopping blood passage from ventricle 23 to the atrium during the systolic cardiac cycle phase.

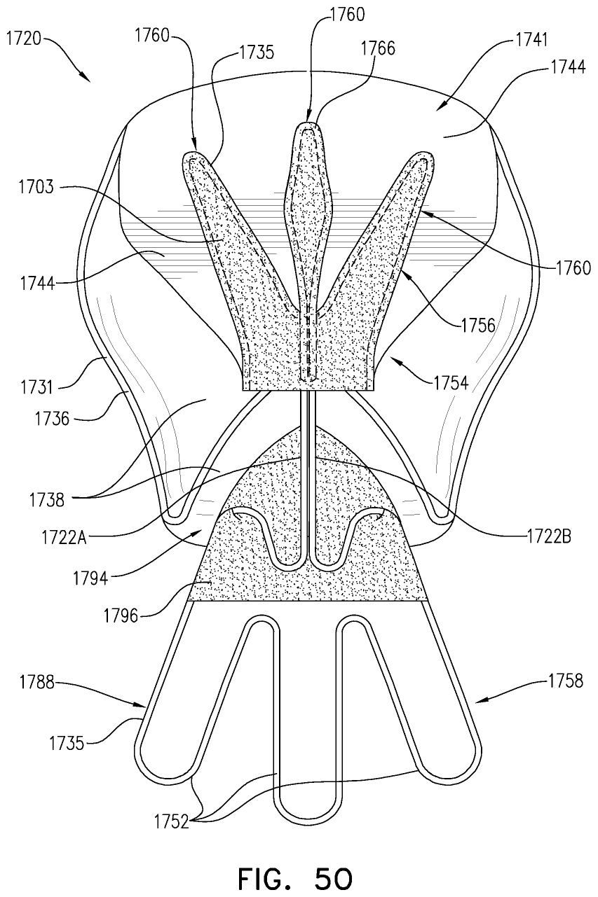

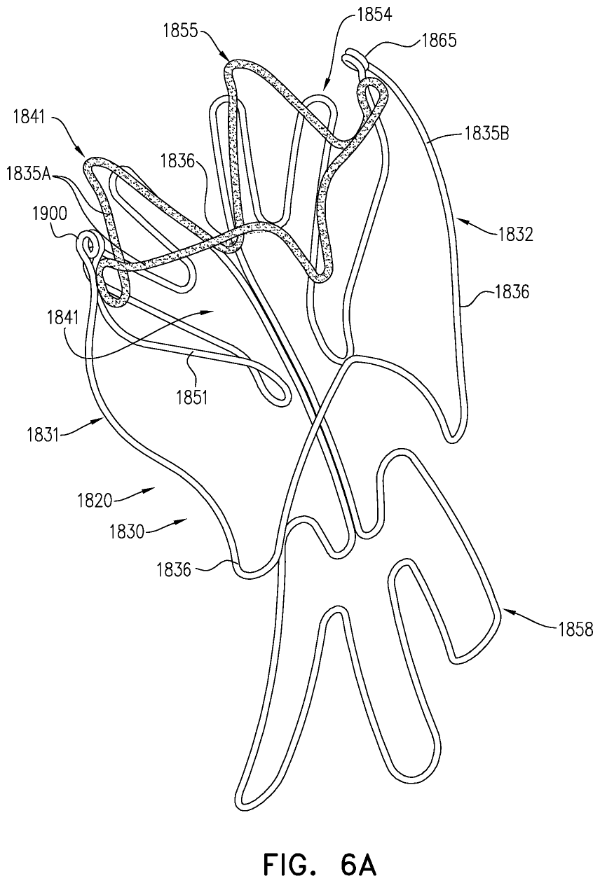

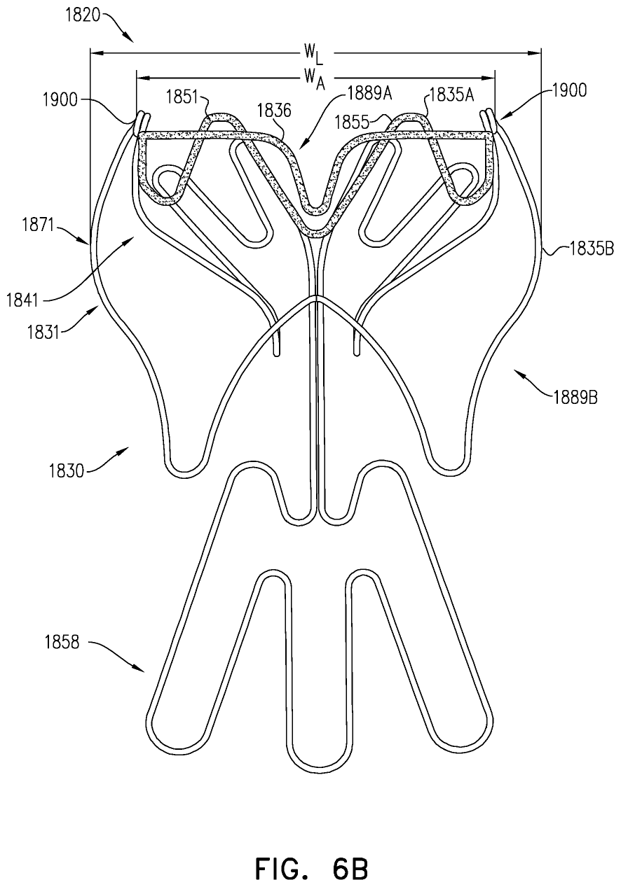

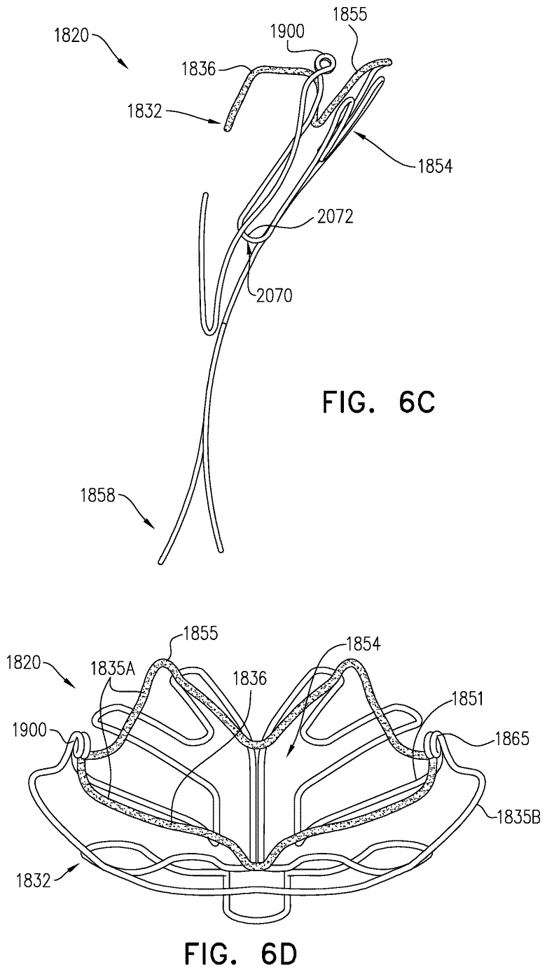

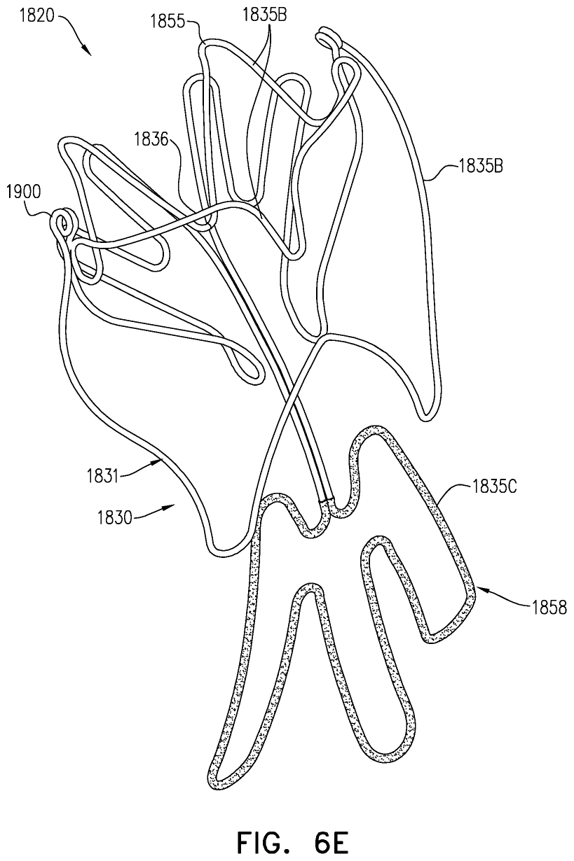

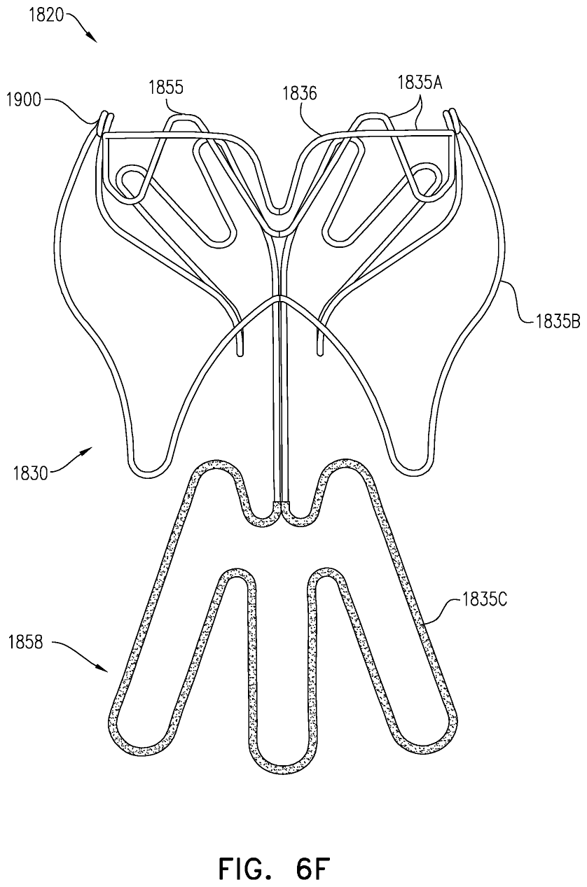

For some applications, neo-leaflet cover 38 has a surface area that is greater than an area defined by and surrounded on approximately three sides by neo-leaflet open wire loop 36, such as shown in and described with reference to FIGS. 6A-C of above-mentioned PCT Publication WO 2020/148755 to Guidotti et al., mutatis mutandis. As a result, when a ventricular surface of neo-leaflet cover 38 is exposed to increased blood pressure during the cardiac cycle, neo-leaflet cover 38 tents away from neo-leaflet open wire loop 36, providing a flexible parachute-like coaptation surface 34 that expand (inflates) and relaxes during the cardiac cycle. Neo-leaflet cover 38 is shown relaxed in FIG. 6A of the '755 publication, and expanded (inflated) by blood flow in FIGS. 6B and 6C of the '755 publication. Optionally, neo-leaflet cover 38 comprises an elastic material.

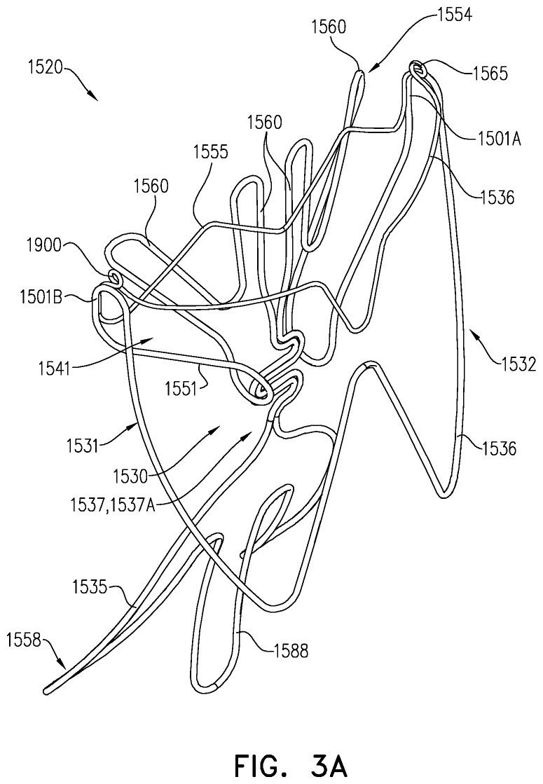

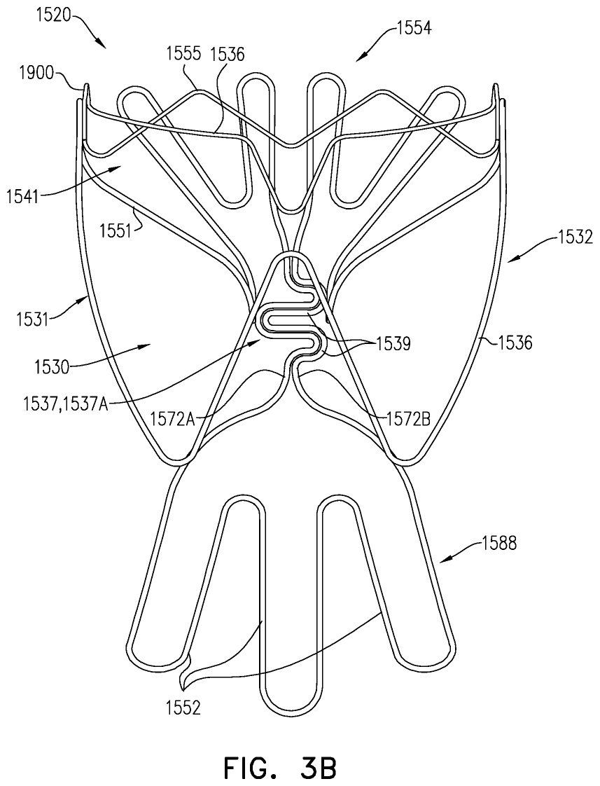

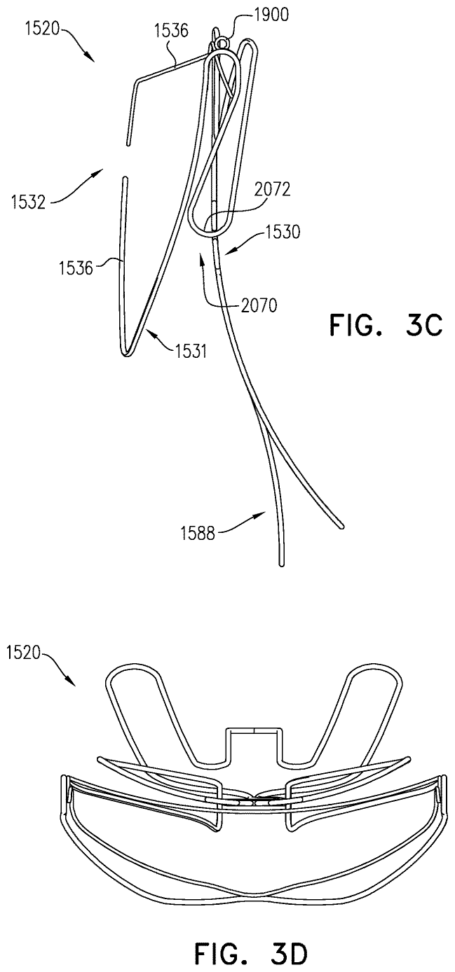

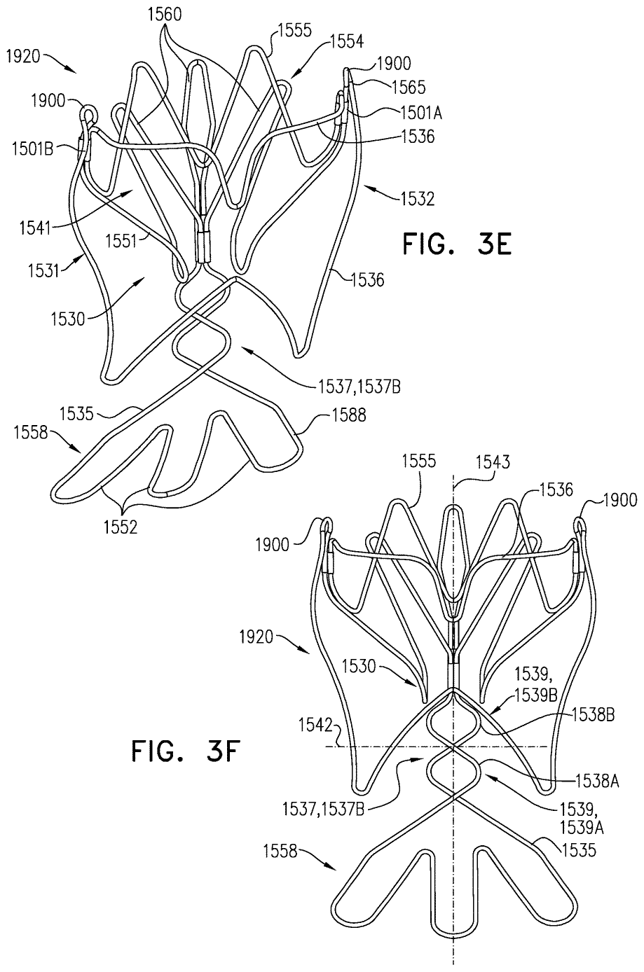

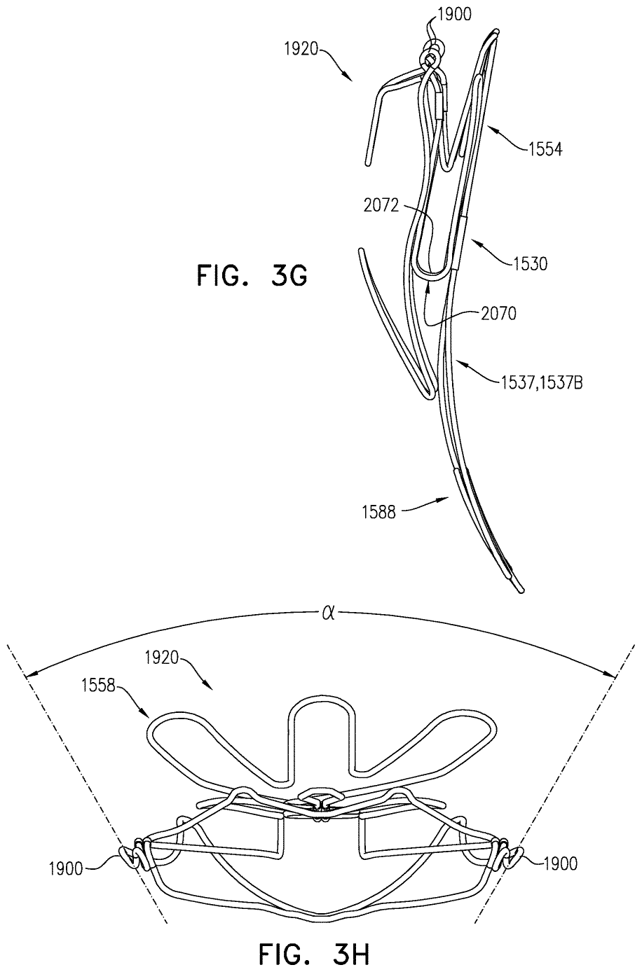

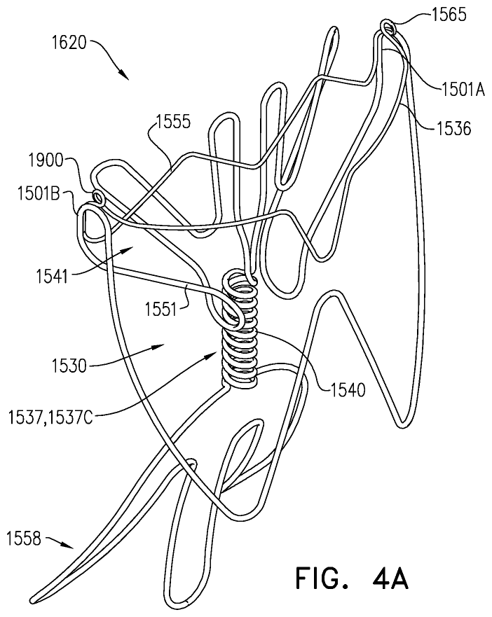

Reference is now made to FIGS. 3A-D, which are schematic illustrations of a coaptation-assist device 1520, in accordance with an application of the present invention.

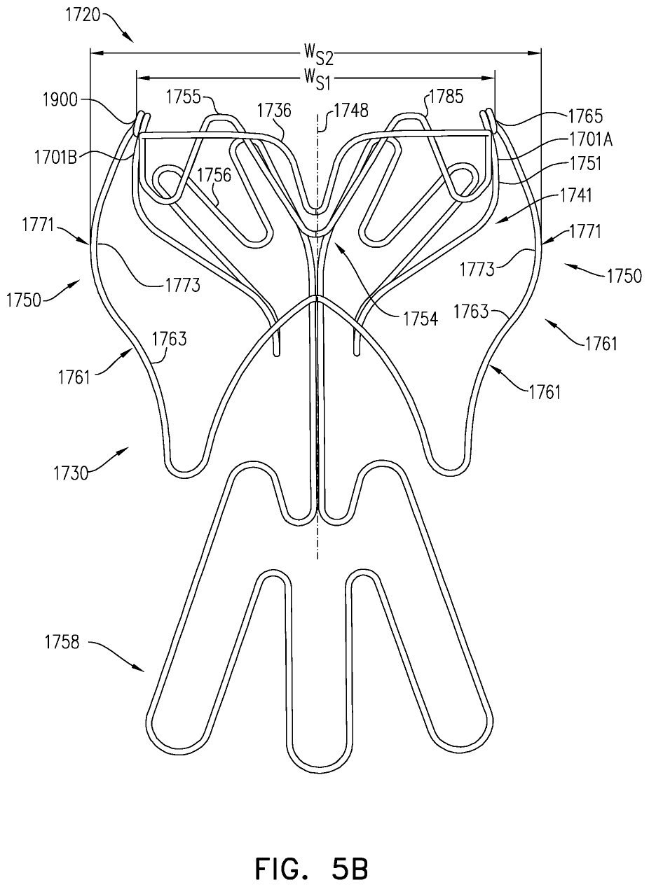

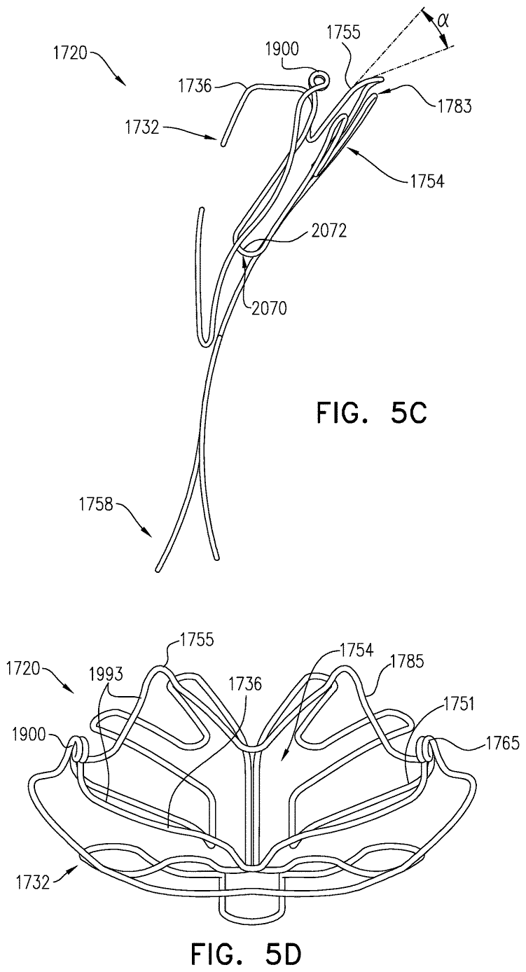

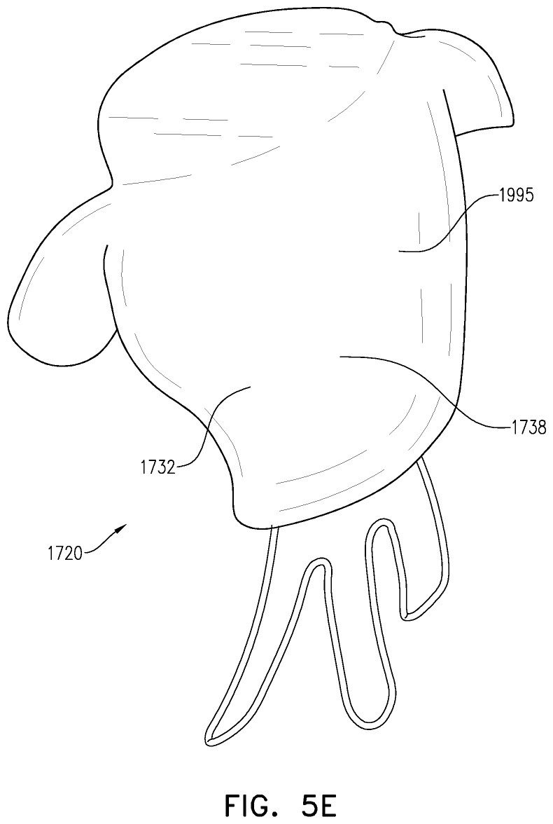

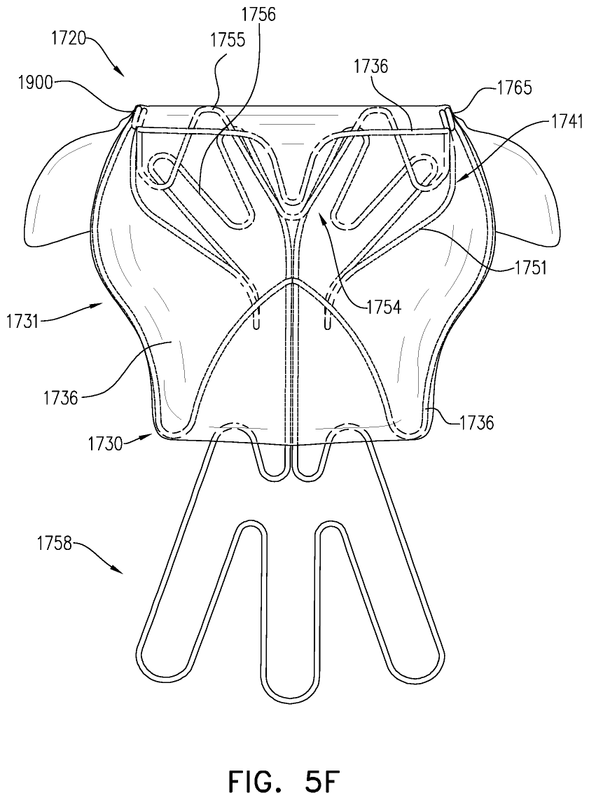

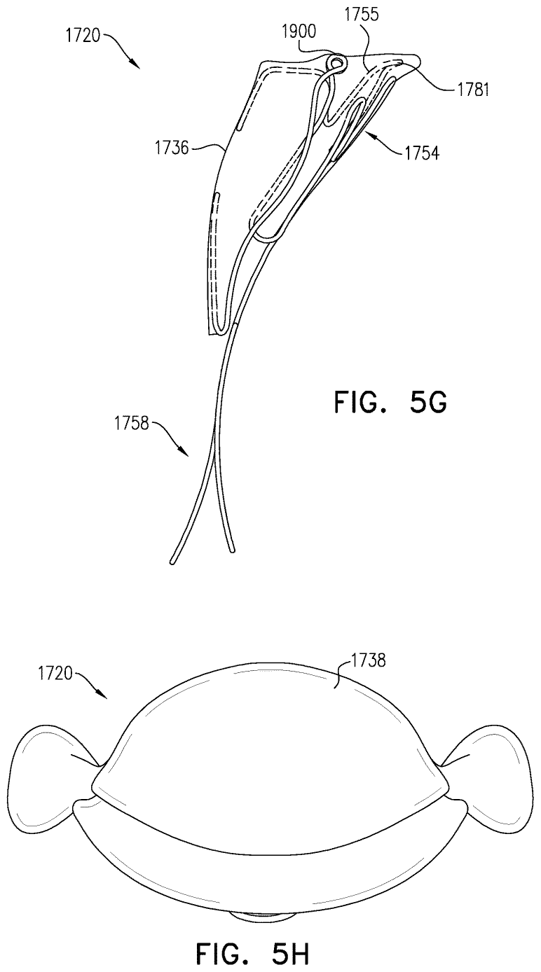

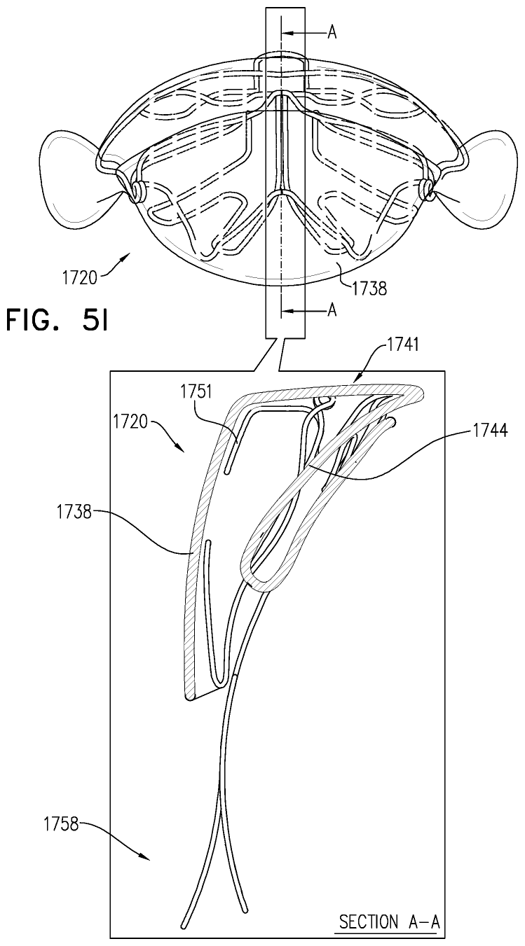

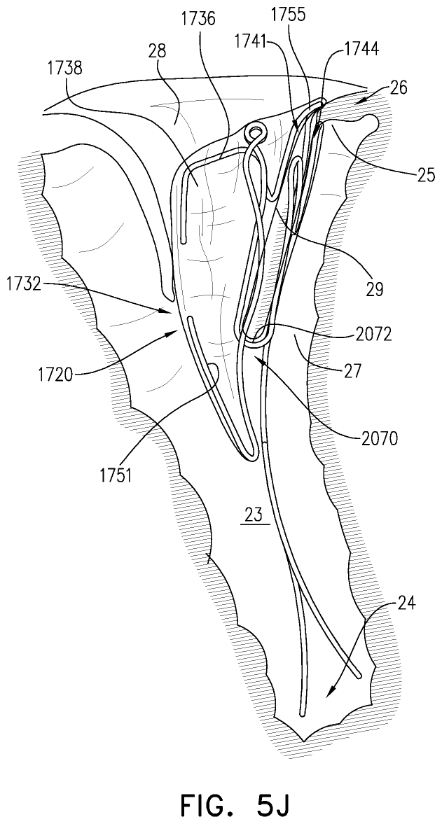

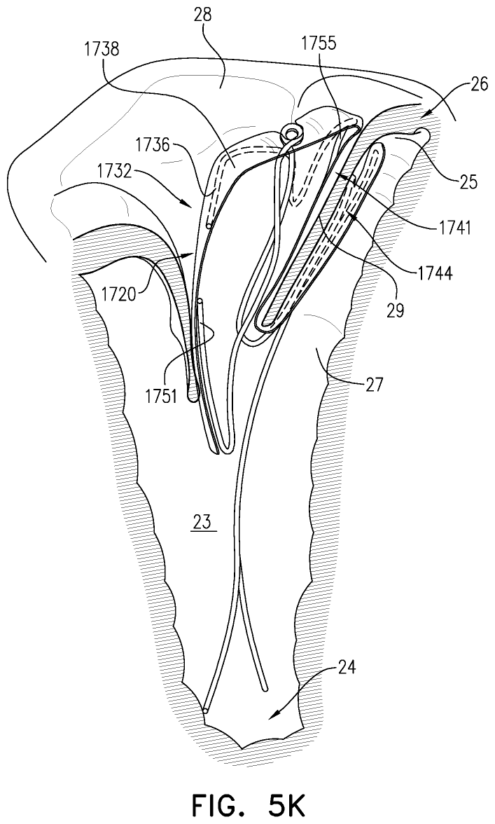

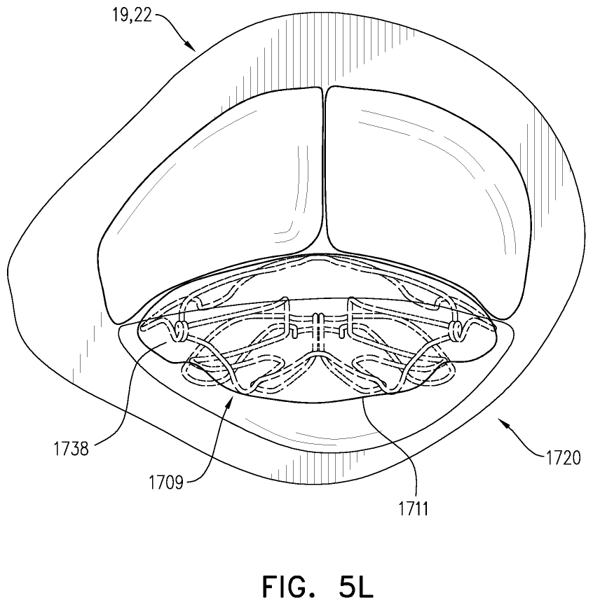

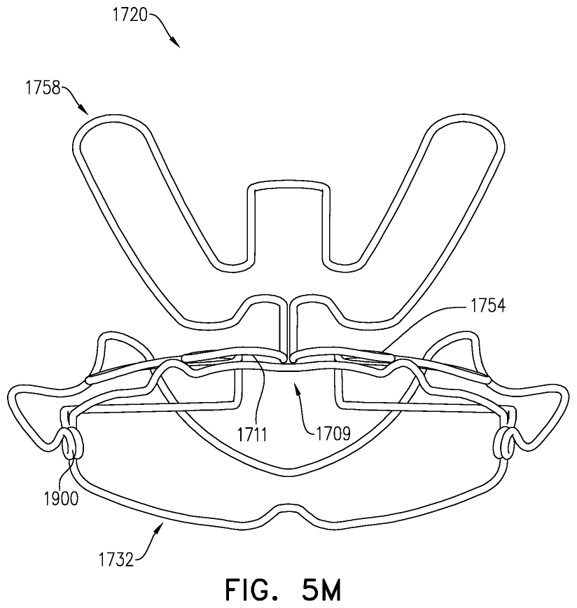

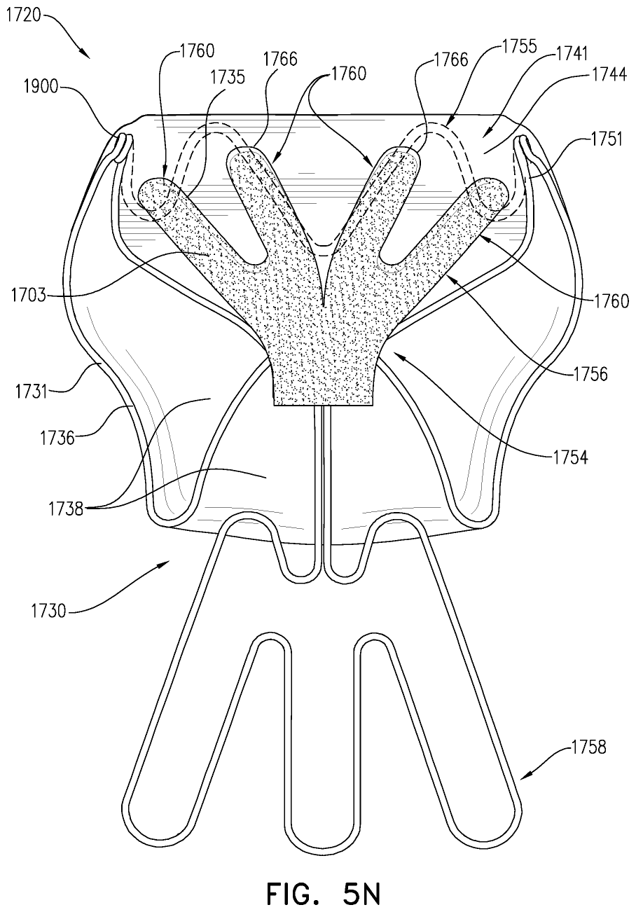

Reference is also made to FIGS. 3E-H, which are schematic illustrations of a coaptation-assist device 1920, in accordance with an application of the present invention. Coaptation-assist device 1920 is similar in some respect to coaptation-assist device 1720, described hereinbelow with reference to FIGS. 5A-O, and like reference numerals refer to like parts.

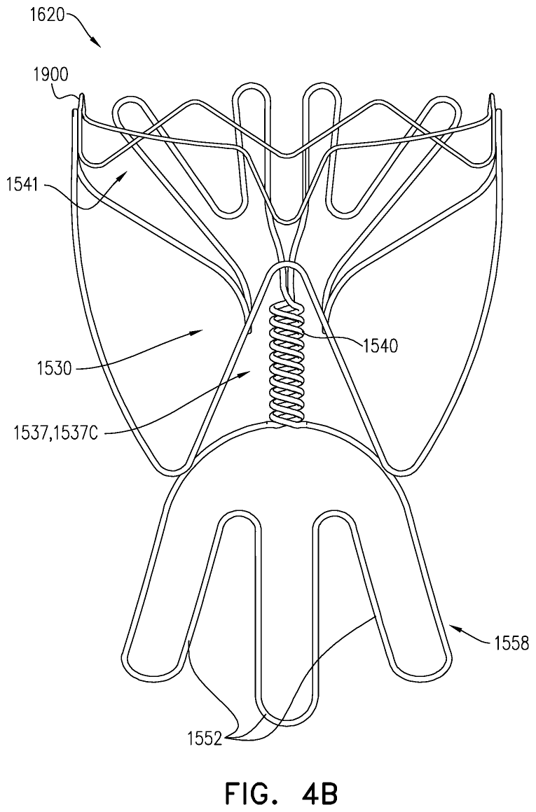

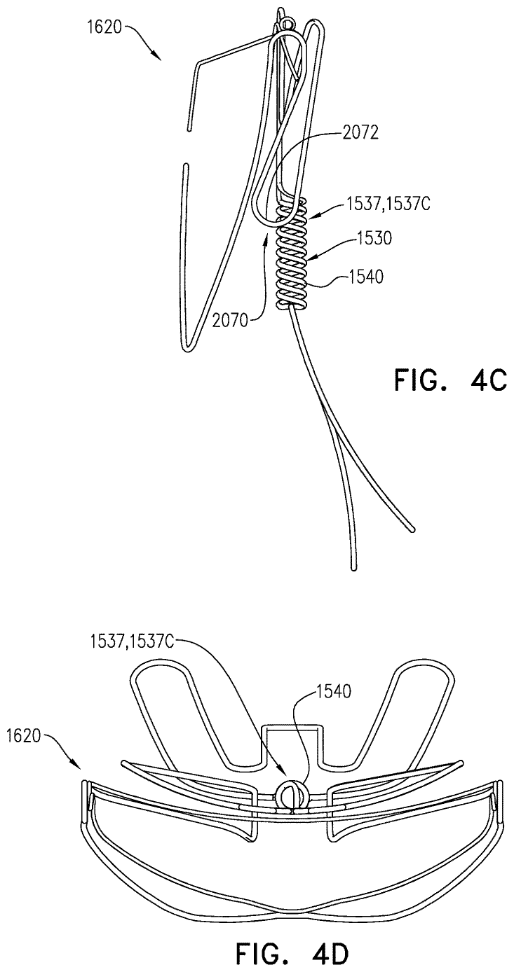

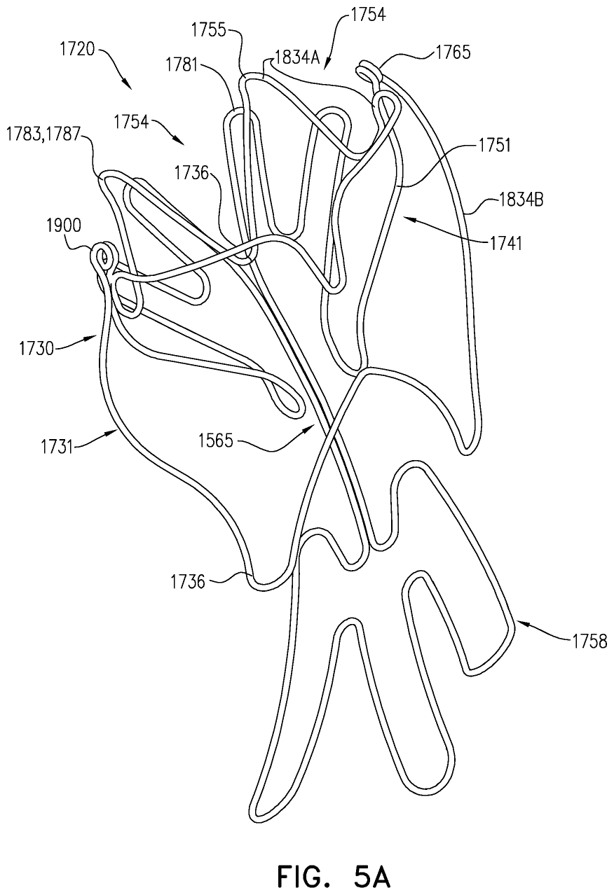

Reference is further made to FIGS. 4A-D, which are schematic illustrations of a coaptation-assist device 1620, in accordance with an application of the present invention. Coaptation-assist devices 1520, 1620, and 1920, including their neo-leaflets and loop-shaped ventricular anchors, may implement (a) any of the features of coaptation-assist device 20, mutatis mutandis, and/or (b) any of the features of the other coaptation-assist devices described herein, mutatis mutandis. Alternatively, coaptation-assist devices 1520, 1620, and 1920, which may be referred to as cardiac assemblies, may not comprise neo-leaflets and may instead comprise a different cardiac treatment device, such as, by way of example and not limitation, cardiac treatment device 2132, 2132A, 2132B described hereinbelow with reference to FIGS. 11A-C, 12, 13, and/or 14.

Each of coaptation-assist devices 1520, 1620, and 1920 comprises a ventricular anchor 1530, which comprises a proximal subannular anchor 1554 and a distal anchor 1558. Ventricular anchor 1530, proximal subannular anchor 1554, and distal anchor 1558 may implement any of the features of the ventricular anchors, proximal subannular anchors, and distal anchors described herein, mutatis mutandis.

Each of coaptation-assist devices 1520, 1620, and 1920 further comprises a neo-leaflet 1532, which may implement any of the features of the neo-leaflets described herein, mutatis mutandis. Neo-leaflet 1532 is supported by ventricular anchor 1530. For some applications, neo-leaflet 1532 comprises a neo-leaflet wire loop 1536, which defines at least a portion of a border 1531 of the neo-leaflet, and a neo-leaflet cover attached to neo-leaflet wire loop 1536. (For clarity of illustration, in FIGS. 3A-D, 3E-H, and 4A-D the coaptation-assist devices are shown without the neo-leaflet cover, as well as without an atrial-surface cover, described hereinabove.) Neo-leaflet wire loop 1536 may implement any of the features of the neo-leaflet wire loops described herein, mutatis mutandis, and the neo-leaflet cover may implement any of the features of the neo-leaflet covers described herein, respectively, mutatis mutandis.

Ventricular anchor 1530 further comprises a springy linking section 1537, which connects proximal subannular anchor 1554 to distal anchor 1558 such that proximal subannular anchor 1554 and distal anchor 1558 can articulate with respect to each other with at least one degree of freedom, such as at least two degrees of freedom, or three degrees of freedom. This articulation may allow for some bending if proximal subannular anchor 1554 (such as fingers 1560 thereof, if provided) is initially not positioned exactly correctly. For example, if lobes 1552, described hereinbelow, are not oriented properly with respect to the annulus, ventricular anchor 1530 can bend towards the commissures in order to position neo-leaflet 1532 correctly on the target leaflet. In addition, if the coaptation-assist device is too long to fit within the ventricle, springy linking section 1537 can compress to reduce the longitudinal length of the coaptation-assist device.

For example, the proximal subannular anchor 1554 and distal anchor 1558 may articulate with respect to each other to allow lateral side bending, axial twisting, and/or antero-posterior bending (such as to increase or decrease the convexity of an angle between subannular anchor 1554 and ventricular anchor 1530). Optionally, the proximal subannular anchor 1554 and distal anchor 1558 may articulate with respect to each other to provide different levels of directionality of bending, e.g., greater bending around one axis than around a second axis.

For some applications, springy linking section 1537 is axially extensible and contractable, so as to provide a variable distance between proximal subannular anchor 1554 and distal anchor 1558. This configuration may allow coaptation-assist devices 1520, 1620, and 1920 to accommodate differing axial lengths of the ventricle.

Reference is made to FIGS. 3A-D and 3E-H. For some applications, springy linking section 1537 comprises a springy linking section 1537A (as shown in FIGS. 3A-D) or a springy linking section 1537B (as shown in FIGS. 3E-H) that is serpentine and is shaped so as to define one or more undulations 1539.

Reference is made to FIGS. 3E-H. For some applications, serpentine springy linking section 1537B is double-serpentine and is defined by first and second wire segments 1538A and 1538B (labeled in FIG. 3F) of the one or more wires 1535 described hereinbelow, such that first wire segment 1538A defines one or more first undulations 1539A of the one or more undulations 1539 and second wire segment 1538B defines one or more second undulations 1539B of the one or more undulations 1539. First and second wire segments 1538A and 1538B run alongside and crisscross each other. Typically, the one or more first undulations 1539A are out of phase with the one or more second undulations 1539B.

For some applications, such as shown in FIGS. 3A-D and 3E-H, and labeled in FIG. 3F, springy linking section 1537 is more flexible about a first axis of rotation 1542 perpendicular to a longitudinal central axis 1543 of springy linking section 1537 than about a second axis of rotation perpendicular to the longitudinal central axis of springy linking section 1537, the second axis perpendicular to first axis 1542 (the second axis is not shown, but is perpendicular to the plane of the sheet of figures).

Reference is made to FIGS. 4A-D. For some applications, springy linking section 1537 comprises a springy linking section 1537C that is shaped so as to define a helix 1540.

Reference is again made to FIGS. 3A-D, 3E-H, and 4A-D. For some applications, ventricular anchor 1530 comprises one or more wires 1535. For some of these applications, springy linking section 1537 is defined at least in part by at least one of the one or more wires 1535. (The one or more wires 1535 comprise first and second wire segments 1538A and 1538B of springy linking section 1537B, described hereinabove with reference to FIGS. 3E-H.)

For some applications, distal anchor 1558 is defined at least in part by at least one of the one or more wires 1535 shaped as a distal wire loop 1588. For some of these applications, the at least one of the one or more wires 1535 that defines distal anchor 1558 further defines springy linking section 1537, and, optionally, at least a portion of proximal subannular anchor 1554.

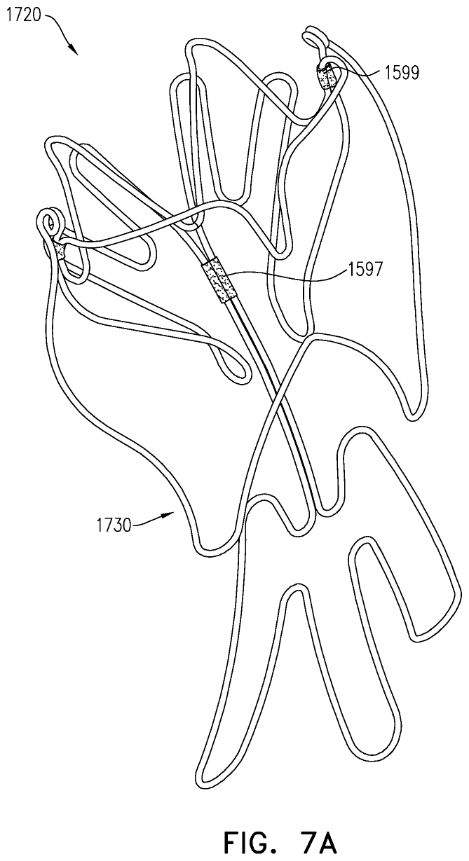

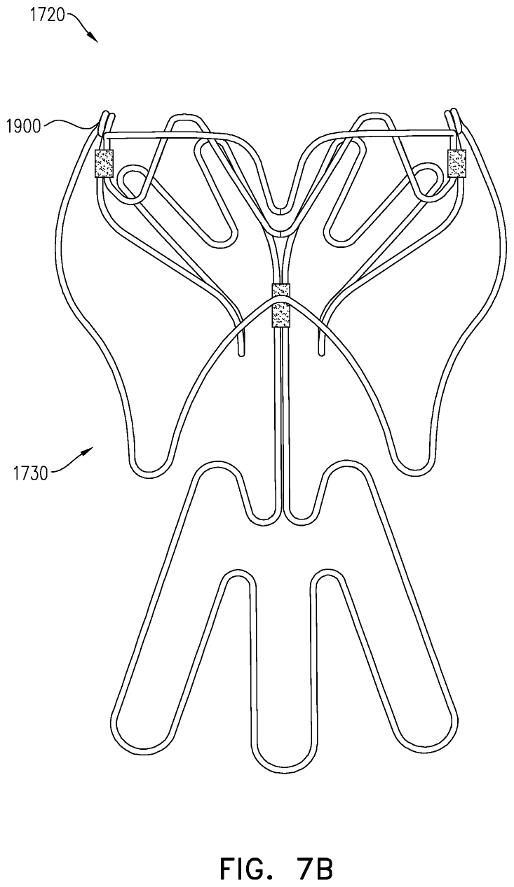

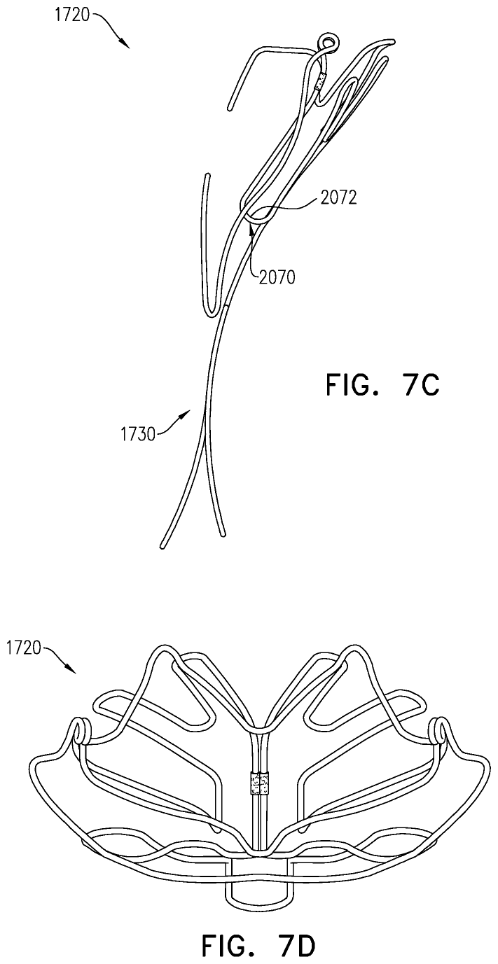

For some applications, such as shown in FIGS. 3A-D, two longitudinal portions 1572A and 1572B of the at least one of the one or more wires 1535 that defines distal anchor 1558 and springy linking section 1537 are fixed to each other. For example, the two longitudinal portions (which partially longitudinally overlap) may be fixed to each other by crimping, such as using a tube (as described hereinbelow with reference to FIGS. 7A-D), a coil, a ligament, or a spiral; and/or by welding, e.g., laser welding.

Reference is still made to FIGS. 3A-D, 3E-H, and 4A-D. For some applications, each of coaptation-assist devices 1520, 1620, and 1920 further comprises an atrial-surface support 1541. Atrial-surface support 1541 and proximal subannular anchor 1554 (e.g., a digitate anchor thereof) are configured to grasp and sandwich at least a portion of target native leaflet 26, in order to support neo-leaflet 1532, and to orient neo-leaflet 1532 with respect to native atrioventricular valve 22.

For some applications, atrial-surface support 1541 comprises a frame 1551 and an atrial-surface cover, which is coupled to frame 1551. The atrial-surface cover is not shown in FIGS. 3A-D, 3E-H, and 4A-D, but is similar to an atrial-surface cover 1744, which is shown in FIGS. 5N and 50, and may implement any of the features of these covers, mutatis mutandis. Frame 1551 comprises one or more wires. Typically, frame 1551 defines at least a portion of a border of atrial-surface support 1541 and/or of the atrial-surface cover.

For some applications, each of coaptation-assist devices 1520, 1620, and 1920 further comprises a proximal supra-leaflet support 1555, which is typically coupled to atrial-surface support 1541. Typically, (a) atrial-surface support 1541 and proximal supra-leaflet support 1555 and (b) proximal subannular anchor 1554 are configured to grasp and sandwich at least a portion of the target native leaflet, in order to support neo-leaflet 1532, and to orient neo-leaflet 1532 with respect to the native atrioventricular valve.

For some applications in which frame 1551 defines at least a portion of the border of atrial-surface support 1541, proximal supra-leaflet support 1555 is coupled to frame 1551 of atrial-surface support 1541, such as to two lateral sides 1501A and 1501B of frame 1551. Optionally, the atrial-surface cover is coupled to proximal supra-leaflet support 1555 in addition to being coupled to frame 1551.

For some applications, as shown, proximal subannular anchor 1554 comprises one or more wires that define the proximal subannular anchor. The one or more wires are configured to be placed below the native leaflet, among the chordae tendineae; to arrive to the base of the native leaflet, and supra-leaflet support 1555; to support the neo-leaflet component between proximal subannular anchor 1554 and proximal supra-leaflet support 1555; and to compress the native leaflet against the atrial surface of the native leaflet once implanted. Proximal supra-leaflet support 1555 comprises one or more wires shaped as to create a compressing structure directed towards the atrial surface of the native leaflet in order to provide a counter element which can compress against proximal subannular anchor 1554.

Reference is still made to FIGS. 3A-D, 3E-H, and 4A-D. For some applications, distal wire loop 1588 is shaped so as to define two or more lobes 1552, e.g., three or more lobes 1552, as shown, or four or more lobes 1552 (configuration not shown). The two or more lobes 1552 may have any of the characteristics of the lobes described herein.

Reference is made to FIGS. 3E-H. As described above, distal anchor 58 and 1558 is configured to be positioned partially against ventricular wall 27 (such as a ventricular septal wall, as mentioned above) outside target subannular space 68, so as to push on the proximal subannular anchor to help anchor the proximal subannular anchor in place within target subannular space 68, mutatis mutandis such as shown in FIG. 2 for coaptation-assist device 20. For some of these applications, such as shown in FIGS. 3E-H, all of lobes 52 and 1552 are configured, when in not constrained, to have the same curvature that generally parallels the curvature of the ventricular septal wall, so as to enhance stability and positioning against the septal wall.

Reference is now made to FIGS. 5A-O, which are schematic illustrations of a coaptation-assist device 1720, in accordance with an application of the present invention. FIGS. 5J-L show coaptation-assist device 1720 implanted in native atrioventricular valve 22. In the particular implantation shown in FIGS. 5J-L, native atrioventricular valve 22 is tricuspid valve 19.

Coaptation-assist device 1720, including its neo-leaflet and loop-shaped ventricular anchor, may implement (a) any of the features of coaptation-assist device 20, mutatis mutandis, and/or (b) any of the features of the other coaptation-assist devices described herein, mutatis mutandis.

Coaptation-assist device 1720 comprises a ventricular anchor 1730, which comprises a proximal subannular anchor 1754 and a distal anchor 1758. Ventricular anchor 1730, proximal subannular anchor 1754, and distal anchor 1758 may implement any of the features of the ventricular anchors, proximal subannular anchors, and distal anchors described herein, mutatis mutandis.

Coaptation-assist device 1720 further comprises a neo-leaflet 1732, which may implement any of the features of the neo-leaflets described herein, mutatis mutandis. Neo-leaflet 1732 is supported by ventricular anchor 1730. For some applications, neo-leaflet 1732 comprises a neo-leaflet wire loop 1736, which defines at least a portion of a border 1731 of the neo-leaflet, and a neo-leaflet cover 1738 attached to neo-leaflet wire loop 1736. (For clarity of illustration, in FIGS. 5A-D and 5M the coaptation-assist device is shown without neo-leaflet cover 1738, as well as without an atrial-surface cover 1744. These covers are shown in FIGS. 5E-L, 5N, and 5O.) Neo-leaflet wire loop 1736 may implement any of the features of the neo-leaflet wire loops described herein, mutatis mutandis, and neo-leaflet cover 1738 may implement any of the features of the neo-leaflet covers described herein, respectively, mutatis mutandis.

For some applications, neo-leaflet 1732 has a central longitudinal axis 1748 that is oriented in a proximal-distal direction. Neo-leaflet wire loop 1736 is shaped so as to define two lateral sides 1750 of neo-leaflet 1732 that face away from central longitudinal axis 1748 and are shaped so as to define respective curved portions 1761 having convex sides 1763 that face toward central longitudinal axis 1748. This shape may reduce the risk of stretching the chordae tendineae of the anterior or the posterior leaflet, which are disposed at that level.

For some applications, the two lateral sides 1750 are not shaped so as to further define any curved portions farther from a junction 1765 between neo-leaflet 1732 and ventricular anchor 1730 than the respective curved portions 1761 are from junction 1765.

For some applications, the respective curved portions 1761 are first respective curved portions 1761, and the two lateral sides 1750 are shaped so as to further define respective second curved portions 1771 having concave sides 1773 that face toward central longitudinal axis 1748, the second curved portions 1771 closer to junction 1765 between neo-leaflet 1732 and ventricular anchor 1730 than the first curved portions 1761 are to junction 1765.

For some applications, the respective curved portions 1761 have respective radii of curvature, each of which is at least 0.25 cm, no more than 5 cm, and/or between 0.25 and 5 cm, such as at least 2 cm, no more than 2.5 cm, and/or between 2 and 2.5 cm.

For some applications, neo-leaflet wire loop 1736 narrows toward junction 1765 between neo-leaflet 1732 and ventricular anchor 1730, for example to avoid stretching of the native annulus.

For some applications, coaptation-assist device 1720 further comprises an atrial-surface support 1741. Atrial-surface support 1741 and proximal subannular anchor 1754 (e.g., a digitate anchor thereof) are configured to grasp and sandwich at least a portion of the target native leaflet, in order to support neo-leaflet 1732, and to orient the neo-leaflet with respect to the native atrioventricular valve. Atrial-surface support 1741 is configured to be disposed below or at a level of an annulus of the native atrioventricular valve and against an atrial surface of the target native leaflet when proximal subannular anchor 1754 is positioned at least partially in the target subannular space.

For some applications, atrial-surface support 1741 comprises a frame 1751 and atrial-surface cover 1744 (shown in FIGS. 5N and 5O), which is coupled to frame 1751. Optionally, atrial-surface cover 1744 is coupled to a surface of frame 1751 that faces proximal subannular anchor 1754. Frame 1751 comprises one or more wires. Typically, frame 1751 defines at least a portion of a border of atrial-surface support 1741 and/or of atrial-surface cover 1744.

For some applications, coaptation-assist device 1720 further comprises a proximal supra-leaflet support 1755, which is coupled to atrial-surface support 1741. Typically, (a) atrial-surface support 1741 and proximal supra-leaflet support 1755 and (b) proximal subannular anchor 1754 are configured to grasp and sandwich at least a portion of the target native leaflet, in order to support neo-leaflet 1732, and to orient neo-leaflet 1732 with respect to the native atrioventricular valve.

For some applications in which frame 1751 defines at least a portion of the border of atrial-surface support 1741, proximal supra-leaflet support 1755 is coupled to frame 1751 of atrial-surface support 1741, such as to two lateral sides 1701A and 1701B of frame 1751 (labeled in FIG. 5B). Optionally, atrial-surface cover 1744 is in contact with proximal supra-leaflet support 1755, as the cover passes over proximal supra-leaflet support 1755 to the neo-leaflet side.

For some applications, proximal supra-leaflet support 1755 extends more proximally than a proximal-most point 1781 of proximal subannular anchor 1754. As a result, atrial-surface support 1741 is typically positioned superior the native annulus upon implantation.

For some applications, one or more proximal-most portions 1783 of proximal supra-leaflet support 1755 are bent toward proximal subannular anchor 1754. As a result, supra-leaflet support 1755 supports neo-leaflet 1732 in order to further cover the upper region of the native annulus. For example, the one or more proximal-most portions 1783 of supra-leaflet support 1755 are bent toward proximal subannular anchor 1754 by an angle a (alpha) (labeled in FIG. 5C) of at least 15 degrees with respect to more distal portions of atrial-surface support 1741, such as at least 30 degrees (and typically no more than 120 degrees, such as no more than 60 degrees).

For some applications, supra-leaflet support 1755 comprises a zig-zag shaped wire 1785. Optionally, zig-zag shaped wire 1785 is shaped so as to define at least two proximal peaks 1787.

For some applications, as labeled in FIG. 5A, a ratio of a greatest width W_{S1} of proximal subannular anchor 1754 (e.g., of a digitate anchor 1756 thereof, such as described hereinabove for other coaptation-assist devices) to a greatest width W_{S2} of neo-leaflet 1732 is less than 140%, such as less than 120%.

Reference is made to FIG. 5M, which is a schematic view of coaptation-assist device 1720 from above, in which supra-leaflet support 1755 and a proximal portion of frame 1751 of atrial-surface support 1741 are not shown for clarity of illustration of the other elements. For some applications, proximal subannular anchor 1754 generally defines a curved surface 1709 having a concave side 1711 that faces toward neo-leaflet 1732. This curvature may generally match the native curvature of the annulus, such that proximal subannular anchor 1754 tracks the curvature of the curvature of the annulus.

For some applications, curved surface 1709 has a radius of curvature of at least 3 cm, no more than 10 cm, and/or between 3 and 10 cm.

For some applications, proximal subannular anchor 1754 comprises digitate anchor 1756, such as described hereinabove for other coaptation-assist devices.

Reference is made to FIG. 5N, which is a schematic view of another configuration of coaptation-assist device 1720, in accordance with an application of the present invention. In this configuration, proximal subannular anchor 1754 comprises digitate anchor 1756 that is defined at least in part by at least one of one or more wires 1735 of ventricular anchor 1730. Digitate anchor 1756 is shaped so as to define a plurality of fingers 1760 typically having a plurality of curved superior peaks 1766. For some applications, digitate anchor 1756 further comprises a digitate cover 1703, which is coupled to one or more of fingers 1760. Digitate cover 1703 is digitate-shaped like fingers 1760, so that digitate cover 1703 can be inserted among chordae tendineae 76 with fingers 1760. Digitate cover 1703 may help reduce the risk of chordae tendineae 76 becoming entangled among the wires that define fingers 1760.

For some applications, digitate cover 1703 comprises one or more biocompatible thin sheets of material, which may comprise a synthetic material or a biological tissue material, such as, for example, a fabric comprising of a polymer or biomaterial (e.g., polyethylene terephthalate (PET), expanded polytetrafluoroethylene (ePTFE), silicone, urethane, or pericardium).

Optionally, a single piece of material is shaped so as to define both digitate cover 1703 and atrial-surface cover 1744.

Reference is now made to FIG. 5O, which is a schematic illustration of another configuration of coaptation-assist device 1720, in accordance with an application of the present invention. This configuration is similar in many respects to the configuration described hereinabove with reference to FIG. 5N. The features of this configuration may optionally be implemented in combination with any of the coaptation-assist devices described herein.

In this configuration, a proximal portion 1794 of distal anchor 1758 comprises a partial anchor covering 1796, which partially covers a distal wire loop 1788 of distal anchor 1758, the distal wire loop defined by one or more wires 1735. Partial anchor covering 1796 may help avoid entanglement of distal anchor 1758 with native chordae or trabecular structures. Partial anchor covering 1796 may have implement any of the features, including materials, of the wire-loop coverings described hereinbelow.

For some applications, partial anchor covering 1796 is shaped generally triangular shape.

For some applications, a portion of partial anchor covering 1796 extends beyond a proximal end of distal anchor 1758.

For some applications, two longitudinal superior portions 1772A and 1772B of distal wire loop 1788 are fixed to each other, and a portion of partial anchor covering 1796 is coupled to the two longitudinal superior portions 1772A and 1772B.

For some applications, distal wire loop 1788 is shaped so as to define two or more lobes 1752, e.g., three or more lobes 1752, as shown, or four or more lobes 1752 (configuration not shown). For some of these applications, partial anchor covering 1796 does not cover lobes 1752, which may allow lobes 1752 to be inserted between native chordae or trabecular structures.

Reference is now made to FIGS. 6A-D, which are schematic illustrations of a coaptation-assist device 1820, in accordance with an application of the present invention. Coaptation-assist device 1820, including its neo-leaflet and loop-shaped ventricular anchor, may implement (a) any of the features of coaptation-assist device 20, mutatis mutandis, and/or (b) any of the features of the other coaptation-assist devices described herein, mutatis mutandis.

Coaptation-assist device 1820 comprises a ventricular anchor 1830, which comprises a proximal subannular anchor 1854 and a distal anchor 1858. Ventricular anchor 1830, proximal subannular anchor 1854, and distal anchor 1858 may implement any of the features of the ventricular anchors, proximal subannular anchors, and distal anchors described herein, mutatis mutandis.

Coaptation-assist device 1820 further comprises a neo-leaflet 1832, which may implement any of the features of the neo-leaflets described herein, mutatis mutandis. Neo-leaflet 1832 is supported by ventricular anchor 1830. For some applications, neo-leaflet 1832 comprises a neo-leaflet wire loop 1836, which defines at least a portion of a border 1831 of the neo-leaflet, and a neo-leaflet cover attached to neo-leaflet wire loop 1836. (For clarity of illustration, in FIGS. 6A-D the coaptation-assist device is shown without the neo-leaflet cover, as well as without an atrial-surface cover, described hereinabove.) Neo-leaflet wire loop 1836 may implement any of the features of the neo-leaflet wire loops described herein, mutatis mutandis, and the neo-leaflet cover may implement any of the features of the neo-leaflet covers described herein, respectively, mutatis mutandis.

Coaptation-assist device 1820 further comprises an atrial-surface support 1841. Atrial-surface support 1841 may implement any of the features of atrial-surface supports 1541 and 1741, described hereinabove with reference to FIGS. 3A-D, 3E-H, 4A-D, and 5A-O, respectively, mutatis mutandis. Atrial-surface support 1841 is configured to be disposed below or at a level of an annulus of the native atrioventricular valve and against an atrial surface of the target native leaflet when proximal subannular anchor 1854 is positioned at least partially in the target subannular space.

Atrial-surface support 1841 and proximal subannular anchor 1854 are configured to grasp and sandwich at least a portion of the target native leaflet, in order to support the neo-leaflet, and to orient the neo-leaflet with respect to the native atrioventricular valve.

For some applications, atrial-surface support 1841 comprises a frame 1851 and an atrial-surface cover, which is coupled to frame 1851. The atrial-surface cover is not shown in FIGS. 6A-D, but is similar to atrial-surface cover 1744, which is shown in FIGS. 5N and 5O, and may implement any of the features of these covers, mutatis mutandis. Frame 1851 comprises one or more wires. Typically, frame 1851 defines at least a portion of a border of atrial-surface support 1841 and/or of the atrial-surface cover.

For some applications, coaptation-assist device 1820 further comprises a proximal supra-leaflet support 1855, which may implement any of the features of the proximal supra-leaflet supports described herein, mutatis mutandis.

For some applications, coaptation-assist device 1820 comprises at least first and second wires 1835A and 1835B. First wire 1835A is shaped so as to define proximal supra-leaflet support 1855 and a proximal first portion 1889A of border 1831 of neo-leaflet 1832. Second wire 1835B is shaped so as to define a second portion 1889B of border 1831 of neo-leaflet 1832.

For some applications, first wire 1835A is thicker than second wire 1835B, such as shown. For example, a cross-sectional area of first wire 1835A may equal at least 150%, such as at least 200%, of a cross-sectional area of second wire 1835B. For example, first wire 1835A may have a diameter of between 0.4 and 0.8 mm, such as 0.6 mm, and/or second wire 1835B may have a diameter of between 0.2 and 0.5 mm, such as 0.4 mm.

For other applications, second wire 1835B is thicker than first wire 1835A (configuration not shown), or first and second wires 1834A and 1835B have the same thickness (as shown, for example, in FIGS. 5A-M for coaptation-assist device 1720).

For some applications, second wire 1835B is shaped so as to additionally define at least a portion of ventricular anchor 1830.

For some applications, a width W_{A} of proximal supra-leaflet support 1855 is less than a width W_{L} of neo-leaflet wire loop 1836, such as less than 80% of the width of neo-leaflet wire loop 1836, such as labeled in FIG. 5A.

Reference is now made to FIGS. 6E-F, which are schematic illustrations of an alternative configuration of coaptation-assist device 1820, in accordance with an application of the present invention. Coaptation-assist device 1820 is described hereinabove with reference to FIGS. 6A-D. The features of this configuration may optionally be implemented in combination with any of the coaptation-assist devices described herein. In this configuration, coaptation-assist device 1820 comprises a third wire 1835C, which is shaped so as to define at least a portion of ventricular anchor 1830. Optionally, third wire 1835C may be thicker than first wire 1835B, such as shown. In this configuration, first wire 1835A is thicker than second wire 1835B, second wire 1835B is thicker than first wire 1835A, or first and second wires 1835A and 1835B have the same thickness.

For some applications, first wire 1835A, second wire 1835B, and/or third wire 1835C are optionally manufactured by laser cutting.

Reference is made to FIGS. 3A-D, 3E-H, 4A-D, 5A-J, and 6A-F. For some applications, coaptation-assist device 1520, 1920, 1620, 1720, or 1820 further comprises one or more proximal loops 1900, such as two or more proximal loops 1900, e.g., exactly two proximal loops 1900, as shown.

For some applications, the one or more proximal loops 1900 are located at a junction 1565, 1765, or 1865, respectively, between neo-leaflet 1532, 1732, or 1832, respectively, and ventricular anchor 1530, 1730, or 1830, respectively.

For some applications, the one or more proximal loops 1900 have a greatest outer dimension of between 2 and 10 mm.

For some applications, the one or more proximal loops 1900 are elliptical or circular.

For some applications, each of the one or more proximal loops 1900 is shaped so as to define at least two turns.

For some applications, coaptation-assist device 1520, 1920, 1620, 1720, or 1820 comprises a wire that is shaped so as to define the one or more proximal loops 1900, at least a portion of the ventricular anchor 1530, 1730, or 1830, respectively, and at least a portion of the border of the neo-leaflet 1532, 1732, or 1832, respectively.

For some applications, a system is provided that comprises coaptation-assist device 1520, 1920, 1620, 1720, or 1820 and further comprises:
a delivery tube in which the coaptation-assist device is removably disposed in a compressed configuration for minimally-invasive or percutaneous delivery to the heart; and
one or more elongate delivery members (such as wires or sutures), which are removably coupled to the one or more proximal loops 1900, respectively.

Providing the one or more proximal loops 1900 may thus allow both pushing and pulling of the coaptation-assist device using the delivery system, rather than just pushing the coaptation-assist device.

For some applications, the system further comprises one or more couplers. The one or more elongate delivery members are removably coupled to the one or more proximal loops 1900, respectively, via the one or more couplers, respectively.

For some applications, the system is configured to deploy the coaptation-assist device out of a distal end opening of the delivery tube. The coaptation-assist device is removably disposed in the delivery tube in the compressed configuration, with the neo-leaflet and the ventricular anchor extending distally from the one or more proximal loops 1900 (either at least partially axially overlapping (alongside) each another, and/or partially axially non-overlapping each other).

Reference is now made to FIGS. 7A-D, which are schematic illustrations of an alternative configuration of coaptation-assist device 1720, in accordance with an application of the present invention. Coaptation-assist device 1720 is described hereinabove with reference to FIGS. 5A-M. The features of this configuration may optionally be implemented in combination with any of the coaptation-assist devices described herein.

As mentioned above with reference to FIGS. 3A-D, for some applications, two longitudinal portions 1572A and 1572B of the at least one of the one or more wires 1535 that defines distal anchor 1558 and springy linking section 1537 are fixed to each other. For example, the two longitudinal portions may be fixed to each other by crimping, such as using a tube 1597.

For some applications, frame 1751 of atrial-surface support 1741 is fixed to proximal subannular anchor 1754, such as by crimping, such as using a tube 1599, a coil, a ligament, or a spiral; and/or by welding, e.g., laser welding.

For some applications, any of the distal wire loops described herein, including with reference to FIGS. 1A-C, 3A-D, 3E-H, 4A-D, 5A-O, 6A-F, 7A-D, 8A-C, 9A-D, 10, 11A-C, 12, and 34, may further comprise a wire-loop covering attached to distal wire loop 88. For some of these applications, the wire-loop covering comprises one or more biocompatible thin sheets of material that extend across and partially or entirely occupy a space at least partially surrounded by the distal wire loop, such as described hereinabove with reference to FIG. 5O and hereinbelow with reference to FIGS. 9A-D. Typically, the one or more biocompatible thin sheets of material are soft and atraumatic. For some applications, the one or more biocompatible thin sheets of material are configured to promote endothelization. The one or more biocompatible thin sheets of material may comprise a synthetic material or a biological tissue material, such as, for example, a fabric comprising a polymer or biomaterial (e.g., polyethylene terephthalate (PET), expanded polytetrafluoroethylene (ePTFE), silicone, urethane, or pericardium), or a balloon.

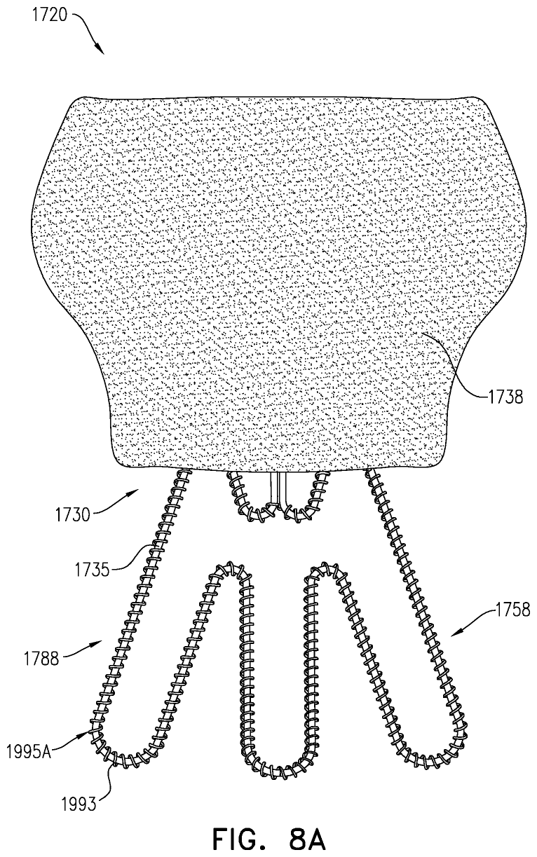

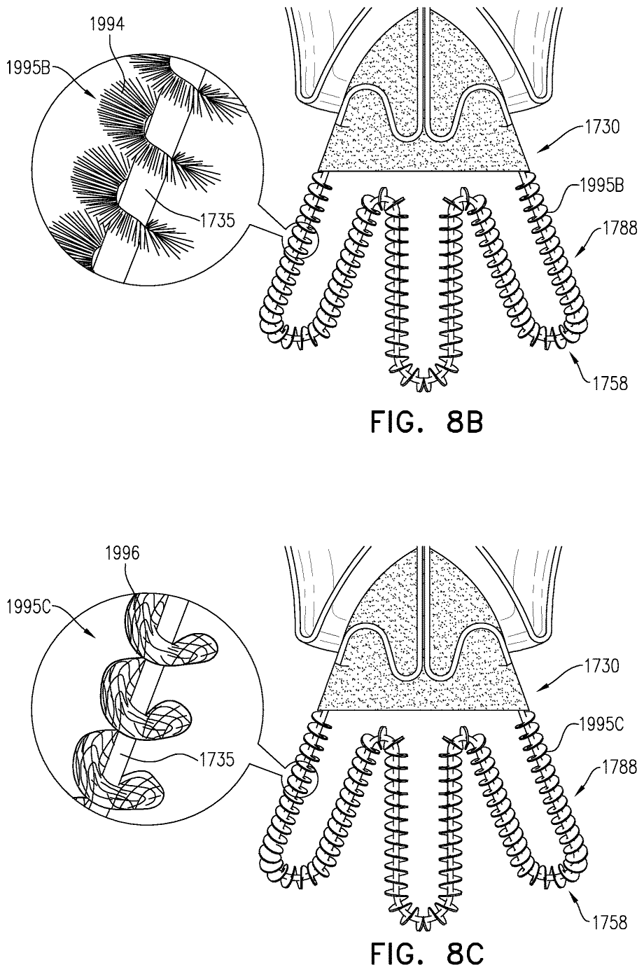

For still other applications, the wire-loop covering comprises a coating on the distal wire loop, e.g., tissue, polyethylene terephthalate (PET), expanded polytetrafluoroethylene (ePTFE), electrospun PTFE, a foam, or a bioabsorbable material. Alternatively or additionally, the distal wire loop surface may be mechanically or chemically treated, e.g., by electropolishing or sandblasting, or provided with barbs, in order to create friction to help maintain the loop in place. Further alternatively or additionally, the one or more wires 35 that are shaped as distal wire loop 88 may be one or more primary wires, and the wire-loop covering may comprise one or more secondary wires that are coiled around the one or more primary wires (such as to increase a volume of the one or more primary wires, such as to create atraumatic contact with ventricular tissue), such as described hereinbelow with reference to FIGS. 8A-C.

Reference is now made to FIGS. 8A-C, which are schematic illustrations of further alternative configurations of coaptation-assist device 1720, in accordance with respective applications of the present invention. Coaptation-assist device 1720 is described hereinabove with reference to FIGS. 5A-O. The features of these configurations may optionally be implemented in combination with any of the coaptation-assist devices described herein.

In the configuration shown in FIG. 8A, the one or more wires 1735 that are shaped as distal wire loop 1788 of distal anchor 1758 are one or more primary wires 1735, and a wire-loop covering 1995A is provided that comprises one or more secondary wires 1993 that are coiled around the one or more primary wires 1735 (such as to increase a volume of the one or more primary wires, such as to create atraumatic contact with ventricular tissue).

In the configuration shown in FIG. 8B, the one or more wires 1735 that are shaped as distal wire loop 1788 of distal anchor 1758 are one or more primary wires 1735, and a wire-loop covering 1995B is provided that comprises brush bristles 1994 that extend radially outward from the one or more primary wires 1735 (such as to increase a volume of the one or more primary wires, such as to create atraumatic contact with ventricular tissue). The combination of the one or more primary wires 1735 and brush bristles 1994 may be similar in some respects to conventional pipe cleaners. For example, brush bristles 1994 may comprise a polymer, such as a synthetic polymer, or a fabric.

In the configuration shown in FIG. 8C, the one or more wires 1735 that are shaped as distal wire loop 1788 of distal anchor 1758 are one or more primary wires 1735, and a wire-loop covering 1995C is provided that comprises a spiral wire brush 1996 that is spiraled around the one or more primary wires 1735 (such as to increase a volume of the one or more primary wires, such as to create atraumatic contact with ventricular tissue). The combination of the one or more primary wires 1735 and spiral wire brush 1996 may be similar in some respects to conventional shotgun barrel cleaner brushes.

Reference is now made to FIGS. 9A-D, which are schematic illustrations of configurations of distal anchor 1758, in accordance with respective applications of the present invention. Although described for distal anchor 1758 of coaptation-assist device 1720, these configurations may also be implemented on the distal anchors of the other coaptation-assist devices described herein, mutatis mutandis. These configurations may optionally be implemented with any of the features of partial anchor covering 1796 described hereinabove with reference to FIG. 5O, mutatis mutandis.

The configuration shown in FIG. 9A is the same as the configuration shown in FIG. 5O, described hereinabove. Proximal portion 1794 of distal anchor 1758 comprises partial anchor covering 1796, which partially covers distal wire loop 1788 of distal anchor 1758, the distal wire loop defined by one or more wires 1735. Partial anchor covering 1796 may help avoid entanglement of distal anchor 1758 with native chordae or trabecular structures.

The configurations shown in FIGS. 9B-D are similar to the configuration shown in FIG. 9A, except as described immediately below.

For some applications, distal wire loop 1788 is shaped so as to define two or more lobes 1752, e.g., three or more lobes 1752, as shown, or four or more lobes 1752 (configuration not shown).

For some of these applications, such as shown in FIG. 9A, partial anchor covering 1796 does not cover any portion of lobes 1752, which may allow lobes 1752 to be inserted between native chordae or trabecular structures.

For others of these applications, such as shown in FIGS. 9B and 9D, partial anchor covering 1796 covers only respective portions of lobes 1752 (such as respective proximal portions, as shown), which may still allow lobes 1752 to be inserted between native chordae or trabecular structures.

For still others of these applications, such as shown in FIG. 9C, an anchor covering 1796B entirely covers lobes 1752 (and typically entirely covers distal wire loop 1788).

For still others of these applications, such as shown in FIG. 9D, partial anchor covering 1796 covers only respective proximal portions of lobes 1752, and separate partial anchor coverings 1796C cover respective distal portions of lobes 1752, which may allow lobes 1752 to be inserted between native chordae or trabecular structures.

As shown in FIGS. 9C-D, the wire-loop covering comprises distal-tip coverings 1789, which cover respective distal tip portions 1791 of respective lobes 1752, in order to increase the atraumaticity of distal tip portions 1791. For example, distal-tip coverings 1789 may comprise foam, silicone, or a rubber-like material, optionally covered by a thin sheet of material. Alternatively or additionally, distal-tip coverings 1789 may comprise extra material of the thin sheet of material that extends across and partially or entirely occupies a space at least partially surrounded by the distal wire loop, as described above.

The configuration of FIG. 9D may be implemented in combination with any of the configurations shown in FIGS. 9A, 9B, and 9C.

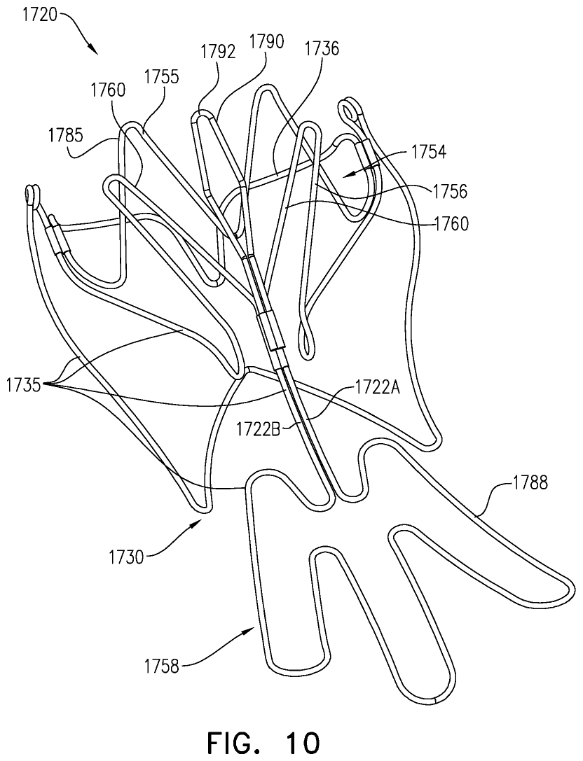

Reference is now made to FIG. 10, which is a schematic illustration of another alternative configuration of coaptation-assist device 1720, in accordance with an application of the present invention. Coaptation-assist device 1720 is described hereinabove with reference to FIGS. 5A-O. The features of this configuration may optionally be implemented in combination with any of the coaptation-assist devices described herein.

In this configuration, proximal subannular anchor 1754 comprises digitate anchor 1756 that is defined at least in part by at least one of one or more wires 1735 of ventricular anchor 1730. Digitate anchor 1756 is shaped so as to define a plurality of fingers 1760. Two longitudinal superior portions 1772A and 1772B of distal wire loop 1788 of coaptation-assist device 1720 are fixed to each other. Digitate anchor 1756 further comprises a separate wire segment 1790, which is coupled to longitudinal superior portions 1772A and 1772B, and is shaped so as to define a central proximal finger 1792 that supplements fingers 1760.

Reference is now made to FIGS. 3A-H, 4A-D, 5A-O, 6A-F, and 7A-D. For some applications, coaptation-assist device 1520, 1620, 1720, 1820, and 1920 is shaped so as to define a junction 2070 between proximal subannular anchor 1554, 1754, 1854 and atrial-surface support 1541, 1741, 1841. For example, junction 2070 may be curved (as shown), or may be straight and/or slanted (configurations not shown). For some applications, junction 2070 is shaped so as to define a proximally-facing inner curvature 2072.

Reference is now made to FIGS. 11A-C and FIG. 12, which are schematic illustrations of a cardiac assembly 2120, 2120A in a disassembled state and an assembled state, respectively, in accordance with an application of the present invention.

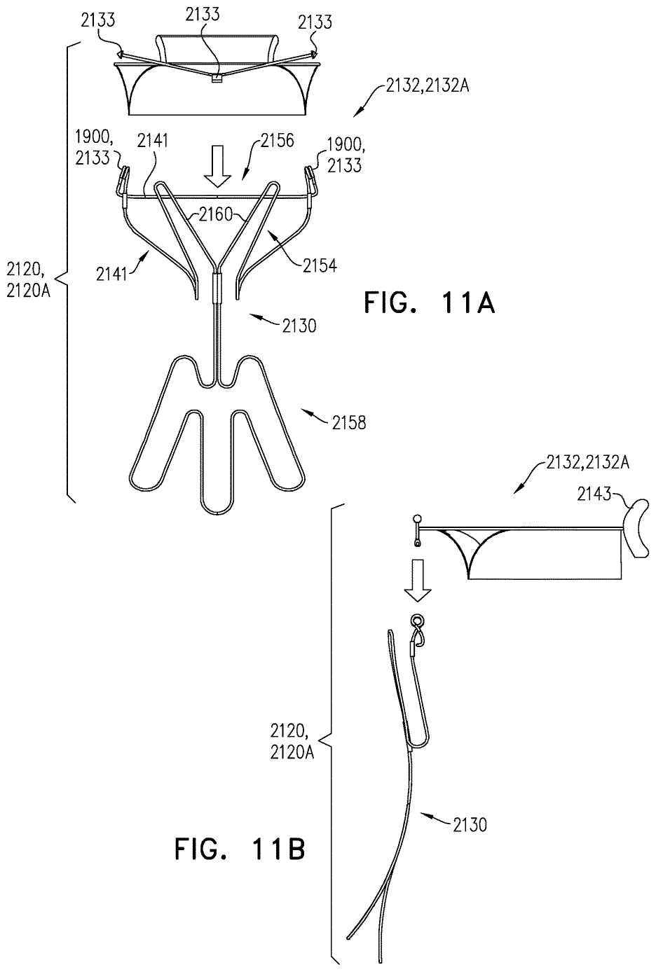

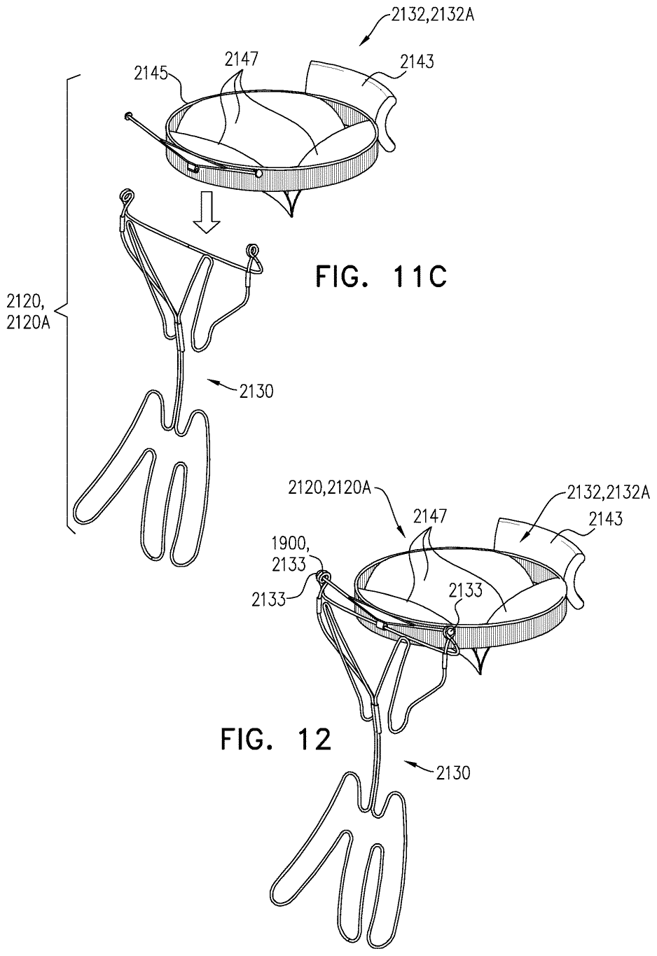

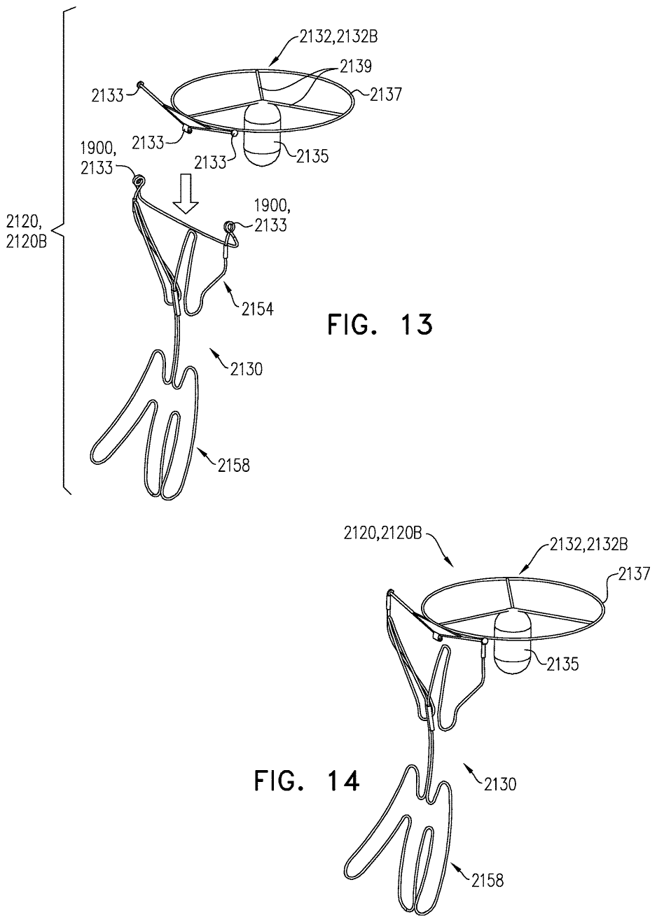

Reference is also made to FIGS. 13 and 14, which are schematic illustrations of a cardiac assembly 2120, 2120B in a disassembled state and an assembled state, respectively, in accordance with an application of the present invention.

Cardiac assembly 2120, 2120A, 2120B comprises a cardiac treatment device 2132, 2132A, 2132B and a ventricular anchor 2130. By way of example and not limitation, ventricular anchor 2130 is shown in FIGS. 11A-14 as being generally similar in some respects to ventricular anchor 1730, described hereinabove with reference to FIGS. 5A-O. Ventricular anchor 2130 may implement any of the features of any of the ventricular anchors described herein, mutatis mutandis.

Cardiac treatment device 2132 is configured to replace or improve function of native atrioventricular valve 22. Ventricular anchor 2130 is configured to be positioned in ventricle 23 so as to support cardiac treatment device 2132, typically in a manner generally similar to the manner in which the other ventricular anchors described herein support the neo-leaflets described herein.

For some applications, ventricular anchor 2130 and cardiac treatment device 2132 comprise separate pieces that are configured to be delivered separately to the heart and to be coupled together in the heart. Typically, ventricular anchor 2130 and/or cardiac treatment device 2132 may comprise one or more couplers 2133 that are configured to couple together ventricular anchor 2130 and cardiac treatment device 2132. For example, the one or more couplers 2133 may be male or female, and may comprise hooks, posts, arrows, or snaps. Optionally, ventricular anchor 2130 comprises one or more proximal loops 1900, such as described hereinabove, which, alternatively or additionally to their function described hereinabove serve as female couplers, into which male couplers of cardiac treatment device 2132 are insertable.

For other applications, ventricular anchor 2130 and cardiac treatment device 2132 are coupled to each other and are configured to be delivered to the heart while coupled together.

For some applications, such as shown in FIGS. 11A-C and 12, cardiac treatment device 2132, 2132A comprises a prosthetic heart valve, which is configured to replace the function of native atrioventricular valve 22. The prosthetic heart valve typically comprises a frame 2145 and a plurality of prosthetic leaflets 2147 (e.g., two or three leaflets) coupled thereto, as is known in the prosthetic heart valve art (and unlike the single-leaflet neo-leaflets described hereinabove). Prosthetic leaflets 2147 are configured to coapt with one another during each cardiac cycle. Optionally, the prosthetic heart valve further comprises a skirt coupled to frame 2145, such as shown in FIGS. 11C and 12.

For example:
frame 2145 may be annular;
frame 2145 may comprise metal, such as Nitinol;
frame 2145 may comprise one or more stent struts; and/or
prosthetic leaflets 2147 may comprise any of the materials that neo-leaflet cover 38 may comprise, such as described hereinabove with reference to FIGS. 1A-C and 2.

For some applications, such as shown in FIGS. 13 and 14, cardiac treatment device 2132, 2132A comprises a spacer 2135, and typically a frame 2137 (e.g., an annular frame) within which spacer 2135 is suspended, such as by radial struts 2139. Spacer 2135 is configured to improve function of native atrioventricular valve 22 by interacting with at least a portion of at least one leaflet of native atrioventricular valve 22 so as to at least partially restrict blood flow through native atrioventricular valve 22 when the valve is closed during each cardiac cycle. Spacer 2135 is typically flexible and/or inflatable. For some applications, spacer 2135 implements some or all of the techniques of the Mitra-Spacer™ System (Cardiosolutions, West Bridgewater, MA, USA), e.g., described in U.S. Pat. No. 9,232,999 to Maurer et al.

As mentioned above, ventricular anchor 2130 may implement any of the features of any of the ventricular anchors described herein, mutatis mutandis. By way of example and not limitation:
ventricular anchor 2130 may comprise a proximal subannular anchor 2154 and a distal anchor 2158, which may implement any of the features of the proximal subannular anchors and distal anchors described herein, mutatis mutandis;
cardiac treatment device 2132 may comprise an atrial-surface support 2141, which may implement any of the features of the atrial-surface supports described herein, mutatis mutandis; and/or
proximal subannular anchor 2154 may comprise a digitate anchor 2156 that is defined at least in part by at least one of one or more wires of ventricular anchor 2130 and shaped so as to define a plurality of fingers 2160 typically having a plurality of curved superior peaks.

For some applications, cardiac treatment device 2132 comprises an anti-tilting element 2143 for stabilizing cardiac treatment device 2132 with a portion of the native atrioventricular valve 22 (above, below, and/or on the cardiac annulus) opposite target native leaflet 26 under which proximal subannular anchor 2154 is disposed. For example, anti-tilting element 2143 may be coupled to the frame of cardiac treatment device 2132. (By way of example and not limitation, anti-tilting element 2143 is shown for cardiac treatment device 2132A, and is not shown for cardiac treatment device 2132B.) The scope of the present invention includes embodiments described in the following applications, which are incorporated herein by reference. In an embodiment, techniques and apparatus described in one or more of the following applications are combined with techniques and apparatus described herein:
US Patent Application Publication 2016/0302917
US Patent Application Publication 2019/0350705
U.S. Provisional Application 62/792,092, filed Jan. 14, 2019
U.S. Provisional Application 62/884,404, filed Aug. 8, 2019
PCT Publication WO 2020/148755 to Guidotti et al.
PCT Publication WO 2021/024217 to Kuck et al.
International Application PCT/IB2020/056926, filed Jul. 22, 2020
U.S. Provisional Application 63/224,465, filed Jul. 22, 2021

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A cardiac assembly for application to a native atrioventricular valve of a heart, the cardiac assembly comprising:
a cardiac treatment device, which is configured to replace or improve function of the native atrioventricular valve; and
a ventricular anchor, which is configured to be positioned in a ventricle of the heart so as to support the cardiac treatment device, and which comprises:
a proximal subannular anchor, which is configured to be positioned at least partially in a target subannular space defined by the target native leaflet and a superior portion of a ventricular wall of the ventricle;
a distal anchor, which is configured to be positioned partially against the ventricular wall outside the target subannular space, so as to push on the proximal subannular anchor to help anchor the proximal subannular anchor in place within the target subannular space; and
a springy linking section, which connects the proximal subannular anchor to the distal anchor such that the proximal subannular anchor and the distal anchor can articulate with respect to each other with at least one degree of freedom.

2. The cardiac assembly according to claim 1, wherein the springy linking section is axially extensible and contractable, so as to provide a variable distance between the proximal subannular anchor and the distal anchor.

3. The cardiac assembly according to claim 1, wherein the springy linking section connects the proximal subannular anchor to the distal anchor such that the proximal subannular anchor and the distal anchor can articulate with respect to each other with at least two degrees of freedom.

4. The cardiac assembly according to claim 1, wherein the springy linking section is more flexible about a first axis of rotation perpendicular to a longitudinal central axis of the springy linking section than about a second axis of rotation perpendicular to the longitudinal central axis of the springy linking section, the second axis perpendicular to the first axis.

5. The cardiac assembly according to claim 1, wherein the springy linking section is serpentine and is shaped so as to define one or more undulations.

6. The cardiac assembly according to claim 5,
wherein the ventricular anchor comprises one or more wires, and wherein the springy linking section is defined at least in part by at least one of the one or more wires,
wherein the serpentine springy linking section is double-serpentine and is defined by first and second wire segments of the one or more wires, such that the first wire segment defines one or more first undulations of the one or more undulations and the second wire segment defines one or more second undulations of the one or more undulations, and
the first and the second wire segments run alongside and crisscross each other.

7. The cardiac assembly according to claim 6, wherein the one or more first undulations are out of phase with the one or more second undulations.

8. The cardiac assembly according to claim 1, wherein the springy linking section is shaped so as to define a helix.

9. The cardiac assembly according to claim 1, wherein the ventricular anchor comprises one or more wires, and wherein the springy linking section is defined at least in part by at least one of the one or more wires.

10. The cardiac assembly according to claim 1, wherein the distal anchor is configured to contact the ventricular wall inferior to the target subannular space.

11. The cardiac assembly according to claim 1, wherein the ventricular anchor is configured to be atraumatic so as not to penetrate tissue of surrounding anatomy.

12. The cardiac assembly according to claim 1, wherein the ventricular anchor is configured to penetrate tissue of surrounding anatomy.

13. The cardiac assembly according to claim 1, wherein the cardiac assembly does not comprise any elements that are configured to penetrate tissue.

14. The cardiac assembly according to claim 1, wherein the ventricular anchor and the cardiac treatment device comprise separate pieces that are configured to be delivered separately to the heart and to be coupled together in the heart.

15. The cardiac assembly according to claim 1, wherein the ventricular anchor and the cardiac treatment device are coupled to each other and are configured to be delivered to the heart while coupled together.

16. The cardiac assembly according to any one of claims 1-15, wherein the ventricular anchor comprises one or more wires, and wherein the distal anchor is defined at least in part by at least one of the one or more wires shaped as a distal wire loop.

17. The cardiac assembly according to claim 16, wherein the at least one of the one or more wires that defines the distal anchor further defines the springy linking section.

18. The cardiac assembly according to claim 17, wherein two longitudinal portions of the at least one of the one or more wires that defines the distal anchor and the springy linking section are fixed to each other.

19. The cardiac assembly according to claim 18, wherein the two longitudinal portions are fixed to each other by crimping.

20. The cardiac assembly according to claim 19, wherein the two longitudinal portions are crimped together by a tube.

21. The cardiac assembly according to claim 16, wherein the distal wire loop is configured to be positioned in the ventricle, extending to a ventricular apical area.

22. The cardiac assembly according to claim 21, wherein the distal wire loop is configured to remain anchored in position against the ventricular wall and one or more other ventricular structures of the ventricular apical area, when the distal anchor is positioned partially against the ventricular wall outside the target subannular space.

23. The cardiac assembly according to claim 22, wherein the one or more other ventricular structures include one or more structures selected from the group consisting of: one or more ventricular papillary muscles, and a moderator band.

24. The cardiac assembly according to claim 21, wherein the distal wire loop is shaped so as to define three or more lobes.

25. The cardiac assembly according to claim 21, wherein the distal wire loop is configured to remain anchored in position by force applied by the distal wire loop to surrounding anatomy.

26. The cardiac assembly according to claim 21, wherein the distal wire loop is configured to remain anchored in position by friction between the distal wire loop and surrounding anatomy.

27. The cardiac assembly according to claim 16, wherein the distal anchor comprises a covering that covers at least a portion of the distal wire loop.

28. The cardiac assembly according to claim 27, wherein the covering comprises a coating that coats the at least a portion of the distal wire loop.

29. The cardiac assembly according to claim 27, wherein the covering comprises a sheet of material that covers the at least a portion of the distal wire loop.

30. The cardiac assembly according to claim 29, wherein the covering entirely covers the distal wire loop.

31. The cardiac assembly according to claim 29, wherein the covering partially covers the distal wire loop.

32. The cardiac assembly according to claim 31, wherein the distal wire loop is shaped so as to define two or more lobes, and wherein the anchor covering does not cover any portion of the lobes.

33. The cardiac assembly according to claim 31, wherein the distal wire loop is shaped so as to define two or more lobes, and wherein the anchor covering covers only respective portions of the lobes.

34. The cardiac assembly according to claim 31, wherein the distal wire loop is shaped so as to define two or more lobes, and wherein the anchor covering entirely covers the lobes.

35. The cardiac assembly according to claim 27,
wherein the one or more wires that are shaped as the distal wire loop are one or more primary wires, and
wherein the covering comprises one or more secondary wires that are coiled around the one or more primary wires.

36. The cardiac assembly according to claim 27,
wherein the one or more wires that are shaped as the distal wire loop are one or more primary wires, and
wherein the covering comprises brush bristles that extend radially outward from the one or more primary wires.

37. The cardiac assembly according to claim 27,
wherein the one or more wires that are shaped as the distal wire loop are one or more primary wires, and
wherein the covering comprises a spiral wire brush that is spiraled around the one or more primary wires.

38. The cardiac assembly according to any one of claims 1-15, wherein the ventricular anchor comprises one or more wires, and wherein a continuous portion of at least one of the one or more wires is shaped so as to define both the distal anchor and the springy linking section.

39. The cardiac assembly according to claim 38, wherein the continuous portion of the at least one of the one or more wires is shaped so as to define the distal anchor, the springy linking section, and at least a portion of the proximal subannular anchor.

40. The cardiac assembly according to claim 38, wherein the continuous portion of the at least one of the one or more wires that defines the distal anchor is shaped as a distal wire loop.

41. The cardiac assembly according to any one of claims 1-15, wherein the proximal subannular anchor is configured such that, when the proximal subannular anchor is positioned at least partially in the target subannular space, the proximal subannular anchor applies a force to a ventricular surface of the target native leaflet, so as to help anchor the ventricular anchor to the target native leaflet.

42. The cardiac assembly according to claim 41,
further comprising an atrial-surface support, which is configured to be disposed below or at a level of an annulus of the native atrioventricular valve and against an atrial surface of the target native leaflet when the proximal subannular anchor is positioned at least partially in the target subannular space,
wherein the atrial-surface support and the proximal subannular anchor are configured to grasp and sandwich at least a portion of the target native leaflet, in order to support the cardiac treatment device, and to orient the cardiac treatment device with respect to the native atrioventricular valve.

43. The cardiac assembly according to claim 42, wherein the atrial-surface support is configured to be disposed below the annulus of the native atrioventricular valve.

44. The cardiac assembly according to claim 42, wherein the atrial-surface support comprises a frame and an atrial-surface cover, which is coupled to the frame.

45. The cardiac assembly according to claim 42,
wherein the cardiac assembly is shaped so as to define a junction between the proximal subannular anchor and the atrial-surface support, and
wherein the cardiac assembly is configured such that the proximal subannular anchor swings outward away from the atrial-surface support upon application of a distally-directed force to the junction while the atrial-surface support is held axially stationary.

46. The cardiac assembly according to any one of claims 1-15, wherein the native atrioventricular valve is a tricuspid valve, and wherein the cardiac treatment device is configured to replace or improve function of the tricuspid valve, when anchored in place by the ventricular anchor.

47. The cardiac assembly according to claim 46, wherein the target native leaflet is a native septal leaflet of the tricuspid valve, and wherein the ventricular wall is a ventricular septal wall.

48. The cardiac assembly according to any one of claims 1-15, wherein the native atrioventricular valve is a mitral valve, and wherein the cardiac treatment device is configured to replace or improve function of the mitral valve, when anchored in place by the ventricular anchor.

49. The cardiac assembly according to claim 48, wherein the target native leaflet is a native anterior leaflet of the mitral valve, and wherein the ventricular wall is a ventricular septal wall.

50. The cardiac assembly according to any one of claims 1-15, wherein the cardiac treatment device comprises a neo-leaflet, which is supported by the ventricular anchor and is configured to at least partially replace function of a target native leaflet of the native atrioventricular valve by providing a surface of coaptation for one or more opposing native leaflets that oppose the target native leaflet, when anchored in place by the ventricular anchor.

51. The cardiac assembly according to claim 50, wherein the neo-leaflet is configured such that the coaptation surface is generally static throughout a cardiac cycle of the heart upon implantation of the cardiac assembly in the heart.

52. The cardiac assembly according to claim 50, wherein the neo-leaflet is configured such that the coaptation surface moves toward and away from the one or more opposing native leaflets during a cardiac cycle of the heart upon implantation of the cardiac assembly in the heart.

53. The cardiac assembly according to claim 50, wherein the coaptation surface has an area of between 2 and 20 cm2.

54. The cardiac assembly according to claim 50, wherein the cardiac assembly is configured such that the coaptation surface of the neo-leaflet crosses from an atrial side to a ventricular side of a native valvular plane, when anchored in place by the ventricular anchor.

55. The cardiac assembly according to claim 50, wherein the neo-leaflet comprises:
a neo-leaflet wire loop that defines at least a portion of a border of the neo-leaflet; and
a neo-leaflet cover, which is attached to the neo-leaflet wire loop and provides the surface of coaptation.

56. The cardiac assembly according to claim 55,
wherein the neo-leaflet has a central longitudinal axis that is oriented in a proximal-distal direction, and
wherein the neo-leaflet wire loop is shaped so as to define two lateral sides of the neo-leaflet that face away from the central longitudinal axis and are shaped so as to define respective curved portions having convex sides that face toward the central longitudinal axis.

57. The cardiac assembly according to claim 56, wherein the two lateral sides are not shaped so as to further define any curved portions farther from a junction between the neo-leaflet and the ventricular anchor than the respective curved portions are from the junction.

58. The cardiac assembly according to claim 56, wherein the respective curved portions are first respective curved portions, and wherein the two lateral sides are shaped so as to further define respective second curved portions having concave sides that face toward the central longitudinal axis, the second curved portions closer to a junction between the neo-leaflet and the ventricular anchor than the first curved portions are to the junction.

59. The cardiac assembly according to claim 56, wherein the respective curved portions have respective radii of curvature, each of which is between 0.25 and 5 cm.

60. The cardiac assembly according to claim 55, wherein the neo-leaflet wire loop narrows toward a junction between the neo-leaflet and the ventricular anchor.

61. The cardiac assembly according to any one of claims 1-15, wherein the cardiac treatment device comprises a prosthetic heart valve, which is configured to replace the function of the native atrioventricular valve, and which comprises a frame and a plurality of prosthetic leaflets that are coupled to the frame and are configured to coapt with one another during each cardiac cycle.

62. The cardiac assembly according to any one of claims 1-15, wherein the cardiac treatment device comprises a spacer, which is configured to improve the function of the native atrioventricular valve by interacting with at least a portion of at least one leaflet of the native atrioventricular valve so as to at least partially restrict blood flow through the native atrioventricular valve when the native atrioventricular valve is closed during each cardiac cycle.

63. The cardiac assembly according to any one of claims 1-15, further comprising at least one pouch, which is configured to inflate by blood flow during a cardiac cycle of the heart, so as to push the cardiac assembly against one or more of: a ventricular surface of the target native leaflet and an annulus of the native atrioventricular valve, thereby stabilizing the cardiac assembly with respect to the native atrioventricular valve.

64. The cardiac assembly according to any one of claims 1-15, wherein the proximal subannular anchor generally defines a curved surface having a concave side that faces toward the cardiac treatment device.

65. The cardiac assembly according to any one of claims 1-15,
wherein the ventricular anchor comprises one or more wires, and
wherein the proximal subannular anchor (a) comprises a digitate anchor that (i) is defined at least in part by at least one of the one or more wires, and (ii) is shaped so as to define a plurality of fingers having a plurality of curved superior peaks, and (b) is configured to be positioned at least partially in the target subannular space such that (i) the curved superior peaks point in a superior direction and engage a subannular surface of the target native leaflet, and (ii) one or more of the fingers engage chordae tendineae that extend between the target native leaflet and the superior portion of the ventricular wall, so as to stabilize the cardiac assembly.

66. The cardiac assembly according to claim 65, wherein the digitate anchor further comprises a digitate cover, which is coupled to one or more of the fingers, and is digitate-shaped like the fingers.

67. A system comprising the cardiac assembly according to any one of claims 1-53, the system further comprising a delivery tube in which the cardiac assembly is removably disposed in a compressed configuration for delivery to the heart.

68. A cardiac assembly for application to a native atrioventricular valve of a heart, the cardiac assembly comprising:
a prosthetic heart valve, which is configured to replace function of the native atrioventricular valve, and which comprises a frame and a plurality of prosthetic leaflets that are coupled to the frame and are configured to coapt with one another during each cardiac cycle; and
a ventricular anchor, which comprises one or more wires, and which is configured be positioned in a ventricle of the heart so as to support the prosthetic heart valve, and which comprises:
a proximal subannular anchor, which (a) comprises a digitate anchor that (i) is defined at least in part by at least one of the one or more wires, (ii) is shaped so as to define a plurality of fingers having a plurality of curved superior peaks, and (b) is configured to be positioned at least partially in a target subannular space defined by the target native leaflet and a superior portion of a ventricular wall of the ventricle, such that (i) the curved superior peaks point in a superior direction and engage a subannular surface of the target native leaflet, and (ii) one or more of the fingers engage chordae tendineae that extend between the target native leaflet and the superior portion of the ventricular wall, so as to stabilize the cardiac assembly, and
a distal anchor, which is configured to be positioned partially against the ventricular wall outside the target subannular space, so as to push on the proximal subannular anchor to help anchor the proximal subannular anchor in place within the target subannular space.

69. The cardiac assembly according to claim 68, wherein the ventricular anchor and the prosthetic heart valve comprise separate pieces that are configured to be delivered separately to the heart and to be coupled together in the heart.

70. The cardiac assembly according to claim 68, wherein the ventricular anchor and the prosthetic heart valve are coupled to each other and are configured to be delivered to the heart while coupled together.

71. A cardiac assembly for application to a native atrioventricular valve of a heart, the cardiac assembly comprising:
a spacer, which is configured to replace improve function of the native atrioventricular valve by interacting with at least a portion of at least one leaflet of the native atrioventricular valve so as to at least partially restrict blood flow through the native atrioventricular valve when the native atrioventricular valve is closed during each cardiac cycle; and
a ventricular anchor, which comprises one or more wires, and which is configured be positioned in a ventricle of the heart so as to support the spacer, and which comprises:
a proximal subannular anchor, which (a) comprises a digitate anchor that (i) is defined at least in part by at least one of the one or more wires, (ii) is shaped so as to define a plurality of fingers having a plurality of curved superior peaks, and (b) is configured to be positioned at least partially in a target subannular space defined by the target native leaflet and a superior portion of a ventricular wall of the ventricle, such that (i) the curved superior peaks point in a superior direction and engage a subannular surface of the target native leaflet, and (ii) one or more of the fingers engage chordae tendineae that extend between the target native leaflet and the superior portion of the ventricular wall, so as to stabilize the cardiac assembly, and
a distal anchor, which is configured to be positioned partially against the ventricular wall outside the target subannular space, so as to push on the proximal subannular anchor help anchor the proximal subannular anchor in place within the target subannular space.

72. The cardiac assembly according to claim 71, wherein the ventricular anchor and the spacer comprise separate pieces that are configured to be delivered separately to the heart and to be coupled together in the heart.

73. The cardiac assembly according to claim 71, wherein the ventricular anchor and the spacer are coupled to each other and are configured to be delivered to the heart while coupled together.

74. The cardiac assembly according to any one of claims 68 and 71, wherein the plurality of curved superior peaks comprises between two and twelve curved superior peaks.

75. The cardiac assembly according to claim 74, wherein the plurality of curved superior peaks comprises between two and twelve curved superior peaks.

76. The cardiac assembly according to claim 75, wherein the plurality of curved superior peaks comprises between four and twelve curved superior peaks.

77. The cardiac assembly according to claim 76, wherein the plurality of curved superior peaks comprises between six and twelve curved superior peaks.

78. The cardiac assembly according to claim 77, wherein the plurality of curved superior peaks comprises between six and eight curved superior peaks.

79. The cardiac assembly according to any one of claims 68 and 71, wherein an average of respective greatest widths of the fingers, measured laterally, is between 2 and 10 mm when the fingers are unconstrained.

80. The cardiac assembly according to any one of claims 68 and 71, wherein a greatest width of a portion of the digitate anchor that defines the fingers is between 20 and 60 mm, when the fingers are unconstrained.

81. The cardiac assembly according to any one of claims 68 and 71, wherein an average of respective distances between midpoints of the curved superior peaks of adjacent pairs of the fingers is between 3 and 9 mm when the fingers are unconstrained.

82. The cardiac assembly according to any one of claims 68 and 71, wherein respective distances between midpoints of the curved superior peaks of adjacent pairs of the fingers vary along a width of the digitate anchor, when the fingers are unconstrained.

83. The cardiac assembly according to any one of claims 68 and 71, wherein all respective distances between midpoints of the curved superior peaks of adjacent pairs of the fingers equal one another, when the fingers are unconstrained.

84. The cardiac assembly according to any one of claims 68 and 71, wherein an average of respective heights of the fingers is between 4 and 30 mm, the respective heights measured in a superior-inferior direction, when the fingers are unconstrained.

85. The cardiac assembly according to any one of claims 68 and 71, wherein an average of respective radii of curvature of the curved superior peaks is between 1 and 10 mm when the fingers are unconstrained.

86. The cardiac assembly according to any one of claims 68 and 71, wherein, for at least one of the fingers, a radius of curvature of the curved superior peak is greater than a smallest width of the finger, measured laterally when the fingers are unconstrained.

87. The cardiac assembly according to any one of claims 68 and 71, wherein when the fingers are unconstrained:
the fingers have respective greatest widths and respective heights, the greatest widths measured laterally, the respective heights measured in a superior-inferior direction,
the fingers have respective ratios of the heights to the greatest widths, and
an average of the respective ratios is between 2 and 5.

88. The cardiac assembly according to any one of claims 68-87, wherein the digitate anchor is serpentine, and is shaped so as to define a plurality of undulations having the plurality of curved superior peaks connected to a plurality of inferior troughs by respective segments.

89. The cardiac assembly according to claim 88, wherein an average of respective greatest widths of the undulations, measured between adjacent pairs of the segments connected by respective curved superior peaks, is between 2 and 10 mm when the undulations are unconstrained.

90. The cardiac assembly according to claim 88, wherein, for at least one of the undulations, a radius of curvature of the curved superior peak is greater than a smallest width of the undulation, measured between adjacent pairs of the segments connected by the curved superior peaks, when the undulations are unconstrained.

91. The cardiac assembly according to claim 88, wherein when the undulations are unconstrained:
the undulations have respective greatest widths and respective heights, the greatest widths measured between adjacent pairs of the segments connected by respective curved peaks, the respective heights measured in a superior-inferior direction,
the undulations have respective ratios of the heights to the greatest widths, and
an average of the respective ratios is between 2 and 5.

92. The cardiac assembly according to any one of claims 68-87, wherein a continuous portion of the one or more wires is shaped so as to define both the distal anchor and the proximal subannular anchor.

93. The cardiac assembly according to claim 92, wherein the portion of the at least one of the one or more wires that defines the distal anchor is shaped as a distal wire loop.

94. The cardiac assembly according to any one of claims 68-87, wherein the proximal subannular anchor is configured such that, when the proximal subannular anchor is positioned at least partially in the target subannular space, the digitate anchor applies a force to a ventricular surface of the target native leaflet, so as to help anchor the ventricular anchor to the target native leaflet.

95. The cardiac assembly according to any one of claims 68-87, wherein the ventricular anchor further comprises a springy linking section, which connects the proximal subannular anchor to the distal anchor such that the proximal subannular anchor and the distal anchor can articulate with respect to each other with at least one degree of freedom.

96. The cardiac assembly according to claim 95, wherein the springy linking section is axially extensible and contractable, so as to provide a variable distance between the proximal subannular anchor and the distal anchor.

97. The cardiac assembly according to claim 95, wherein the springy linking section connects the proximal subannular anchor to the distal anchor such that the proximal subannular anchor and the distal anchor can articulate with respect to each other with at least two degrees of freedom.

98. A system comprising the cardiac assembly according to any one of claims 68-87, the system further comprising a delivery tube in which the cardiac assembly is removably disposed in a compressed configuration for delivery to the heart.

99. A cardiac assembly for application to a native atrioventricular valve of a heart, the cardiac assembly comprising:
a cardiac treatment device, which is configured to replace or improve function of the native atrioventricular valve; and
a ventricular anchor, which comprises one or more wires, and which is configured to be positioned in a ventricle of the heart so as to support the cardiac treatment device, and which comprises:
a proximal subannular anchor, which (a) comprises a digitate anchor that (i) is defined at least in part by at least one of the one or more wires, (ii) is shaped so as to define a plurality of fingers having a plurality of curved superior peaks, and (iii) comprises a digitate cover, which is coupled to one or more of the fingers, and is digitate-shaped like the fingers, and (b) is configured to be positioned at least partially in a target subannular space defined by the target native leaflet and a superior portion of a ventricular wall of the ventricle, such that (i) the curved superior peaks point in a superior direction and engage a subannular surface of the target native leaflet, and (ii) one or more of the fingers engage chordae tendineae that extend between the target native leaflet and the superior portion of the ventricular wall, so as to stabilize the cardiac assembly, and
a distal anchor, which is configured to be positioned partially against the ventricular wall outside the target subannular space, so as to push on the proximal subannular anchor to help anchor the proximal subannular anchor in place within the target subannular space.

100. The cardiac assembly according to claim 99, wherein the plurality of curved superior peaks comprises between two and twelve curved superior peaks.

101. The cardiac assembly according to claim 100, wherein the plurality of curved superior peaks comprises between two and twelve curved superior peaks.

102. The cardiac assembly according to claim 101, wherein the plurality of curved superior peaks comprises between four and twelve curved superior peaks.

103. The cardiac assembly according to claim 102, wherein the plurality of curved superior peaks comprises between six and twelve curved superior peaks.

104. The cardiac assembly according to claim 103, wherein the plurality of curved superior peaks comprises between six and eight curved superior peaks.

105. The cardiac assembly according to claim 99, wherein an average of respective greatest widths of the fingers, measured laterally, is between 2 and 10 mm when the fingers are unconstrained.

106. The cardiac assembly according to claim 99, wherein a greatest width of a portion of the digitate anchor that defines the fingers is between 20 and 60 mm, when the fingers are unconstrained.

107. The cardiac assembly according to claim 99, wherein an average of respective distances between midpoints of the curved superior peaks of adjacent pairs of the fingers is between 3 and 9 mm when the fingers are unconstrained.

108. The cardiac assembly according to claim 99, wherein respective distances between midpoints of the curved superior peaks of adjacent pairs of the fingers vary along a width of the digitate anchor, when the fingers are unconstrained.

109. The cardiac assembly according to claim 99, wherein all respective distances between midpoints of the curved superior peaks of adjacent pairs of the fingers equal one another, when the fingers are unconstrained.

110. The cardiac assembly according to claim 99, wherein an average of respective heights of the fingers is between 4 and 30 mm, the respective heights measured in a superior-inferior direction, when the fingers are unconstrained.

111. The cardiac assembly according to claim 99, wherein an average of respective radii of curvature of the curved superior peaks is between 1 and 10 mm when the fingers are unconstrained.

112. The cardiac assembly according to claim 99, wherein, for at least one of the fingers, a radius of curvature of the curved superior peak is greater than a smallest width of the finger, measured laterally when the fingers are unconstrained.

113. The cardiac assembly according to claim 99, wherein when the fingers are unconstrained:
the fingers have respective greatest widths and respective heights, the greatest widths measured laterally, the respective heights measured in a superior-inferior direction,
the fingers have respective ratios of the heights to the greatest widths, and
an average of the respective ratios is between 2 and 5.

114. The cardiac assembly according to any one of claims 99-113, wherein the cardiac treatment device comprises a neo-leaflet, which is supported by the ventricular anchor and is configured to at least partially replace function of a target native leaflet of the native atrioventricular valve by providing a surface of coaptation for one or more opposing native leaflets that oppose the target native leaflet, when anchored in place by the ventricular anchor.

115. The cardiac assembly according to claim 114, wherein at least half of the fingers terminate superiorly to an inferior edge of the surface of coaptation of the neo-leaflet.

116. The cardiac assembly according to claim 115, wherein all of the fingers terminate superiorly to the inferior edge of the surface of coaptation of the neo-leaflet.

117. The cardiac assembly according to any one of claims 99-113, wherein the cardiac treatment device comprises a prosthetic heart valve, which is configured to replace the function of the native atrioventricular valve, and which comprises a frame and a plurality of prosthetic leaflets that are coupled to the frame and are configured to coapt with one another during each cardiac cycle.

118. The cardiac assembly according to any one of claims 99-113, wherein the cardiac treatment device comprises a spacer, which is configured to improve the function of the native atrioventricular valve by interacting with at least a portion of at least one leaflet of the native atrioventricular valve so as to at least partially restrict blood flow through the native atrioventricular valve when the native atrioventricular valve is closed during each cardiac cycle.

119. The cardiac assembly according to any one of claims 99-113, wherein the digitate anchor is serpentine, and is shaped so as to define a plurality of undulations having the plurality of curved superior peaks connected to a plurality of inferior troughs by respective segments.

120. The cardiac assembly according to claim 119, wherein an average of respective greatest widths of the undulations, measured between adjacent pairs of the segments connected by respective curved superior peaks, is between 2 and 10 mm when the undulations are unconstrained.

121. The cardiac assembly according to claim 119, wherein, for at least one of the undulations, a radius of curvature of the curved superior peak is greater than a smallest width of the undulation, measured between adjacent pairs of the segments connected by the curved superior peaks, when the undulations are unconstrained.

122. The cardiac assembly according to claim 119, wherein when the undulations are unconstrained:
the undulations have respective greatest widths and respective heights, the greatest widths measured between adjacent pairs of the segments connected by respective curved peaks, the respective heights measured in a superior-inferior direction,
the undulations have respective ratios of the heights to the greatest widths, and
an average of the respective ratios is between 2 and 5.

123. The cardiac assembly according to claim 119,
wherein the cardiac treatment device comprises a neo-leaflet, which is supported by the ventricular anchor and is configured to at least partially replace function of a target native leaflet of the native atrioventricular valve by providing a surface of coaptation for one or more opposing native leaflets that oppose the target native leaflet, when anchored in place by the ventricular anchor, and
wherein at least half of the inferior troughs terminate superiorly to an inferior edge of the surface of coaptation of the neo-leaflet.

124. The cardiac assembly according to claim 123, wherein all of the inferior troughs terminate superiorly to the inferior edge of the surface of coaptation of the neo-leaflet.

125. The cardiac assembly according to any one of claims 99-113, wherein a continuous portion of the one or more wires is shaped so as to define both the distal anchor and the proximal subannular anchor.

126. The cardiac assembly according to claim 125, wherein the portion of the at least one of the one or more wires that defines the distal anchor is shaped as a distal wire loop.

127. The cardiac assembly according to any one of claims 99-113, wherein the proximal subannular anchor is configured such that, when the proximal subannular anchor is positioned at least partially in the target subannular space, the digitate anchor applies a force to a ventricular surface of the target native leaflet, so as to help anchor the ventricular anchor to the target native leaflet.

128. A system comprising the cardiac assembly according to any one of claims 99-113, the system further comprising a delivery tube in which the cardiac assembly is removably disposed in a compressed configuration for delivery to the heart.

129. A cardiac assembly for application to a native atrioventricular valve of a heart, the cardiac assembly comprising:
a cardiac treatment device, which is configured to replace or improve function of the native atrioventricular valve; and
a ventricular anchor, which is configured to be positioned in a ventricle of the heart so as to support the cardiac treatment device, and which comprises:
a proximal subannular anchor, which is configured to be positioned at least partially in a target subannular space defined by the target native leaflet and a superior portion of a ventricular wall of the ventricle; and
a distal anchor, which (a) is defined at least in part by at least one of the one or more wires shaped as a distal wire loop, (b) comprises a wire-loop covering that covers at least a portion of the distal wire loop, and (c) is configured to be positioned partially against the ventricular wall outside the target subannular space, so as to push on the proximal subannular anchor to help anchor the proximal subannular anchor in place within the target subannular space.

130. The cardiac assembly according to claim 129, wherein the wire-loop covering comprises a coating that coats the at least a portion of the distal wire loop.

131. The cardiac assembly according to claim 129, wherein the wire-loop covering comprises a sheet of material that covers the at least a portion of the distal wire loop.

132. The cardiac assembly according to claim 129,
wherein the one or more wires that are shaped as the distal wire loop are one or more primary wires, and
wherein the wire-loop covering comprises a secondary wire that is coiled around the one or more primary wires.

133. The cardiac assembly according to claim 129, wherein the ventricular anchor and the cardiac treatment device comprise separate pieces that are configured to be delivered separately to the heart and to be coupled together in the heart.

134. The cardiac assembly according to claim 129, wherein the ventricular anchor and the cardiac treatment device are coupled to each other and are configured to be delivered to the heart while coupled together.

135. The cardiac assembly according to any one of claims 129-134, wherein the distal wire loop is configured to be positioned in the ventricle, extending to a ventricular apical area.

136. The cardiac assembly according to claim 135, wherein the distal wire loop is configured to remain anchored in position against the ventricular wall and one or more other ventricular structures of the ventricular apical area, when the distal anchor is positioned partially against the ventricular wall outside the target subannular space.

137. The cardiac assembly according to claim 136, wherein the one or more other ventricular structures include one or more structures selected from the group consisting of:
one or more ventricular papillary muscles, and a moderator band.

138. The cardiac assembly according to claim 135, wherein the distal wire loop is shaped so as to define three or more lobes.

139. The cardiac assembly according to claim 135, wherein the distal wire loop is configured to remain anchored in position by force applied by the distal wire loop to surrounding anatomy.

140. The cardiac assembly according to claim 135, wherein the distal wire loop is configured to remain anchored in position by friction between the distal wire loop and surrounding anatomy.

141. The cardiac assembly according to any one of claims 129-134, wherein a continuous portion of the one or more wires is shaped so as to define both the distal wire loop and at least a portion of the proximal subannular anchor.

142. The cardiac assembly according to any one of claims 129-134, wherein the cardiac treatment device comprises a neo-leaflet, which is supported by the ventricular anchor and is configured to at least partially replace function of a target native leaflet of the native atrioventricular valve by providing a surface of coaptation for one or more opposing native leaflets that oppose the target native leaflet, when anchored in place by the ventricular anchor.

143. The cardiac assembly according to any one of claims 129-134, wherein the cardiac treatment device comprises a prosthetic heart valve, which is configured to replace the function of the native atrioventricular valve, and which comprises a frame and a plurality of prosthetic leaflets that are coupled to the frame and are configured to coapt with one another during each cardiac cycle.

144. The cardiac assembly according to any one of claims 129-134, wherein the cardiac treatment device comprises a spacer, which is configured to improve the function of the native atrioventricular valve by interacting with at least a portion of at least one leaflet of the native atrioventricular valve so as to at least partially restrict blood flow through the native atrioventricular valve when the native atrioventricular valve is closed during each cardiac cycle.

145. A cardiac assembly for application to a native atrioventricular valve of a heart, the cardiac assembly comprising:
a ventricular anchor, which is configured to be positioned in a ventricle of the heart, and which comprises:
(i) a proximal subannular anchor, which is configured to be positioned at least partially in a target subannular space defined by the target native leaflet and a superior portion of a ventricular wall of the ventricle; and
(ii) a distal anchor, which is configured to be positioned partially against the ventricular wall outside the target subannular space, so as to push on the proximal subannular anchor to help anchor the proximal subannular anchor in place within the target subannular space;
a cardiac treatment device, which is configured to replace or improve function of the native atrioventricular valve;
an atrial-surface support, which is configured to be disposed below or at a level of an annulus of the native atrioventricular valve and against an atrial surface of the target native leaflet when the proximal subannular anchor is positioned at least partially in the target subannular space; and
a proximal supra-leaflet support, which is coupled to the atrial-surface support,
wherein (a) the atrial-surface support and the proximal supra-leaflet support and (b) the proximal subannular anchor are configured to grasp and sandwich at least a portion of the target native leaflet, in order to support the cardiac treatment device, and to orient the cardiac treatment device with respect to the native atrioventricular valve.

146. The cardiac assembly according to claim 145, wherein the atrial-surface support is configured to be disposed below the annulus of the native atrioventricular valve.

147. The cardiac assembly according to claim 145, wherein the distal anchor is configured to contact the ventricular wall inferior to the target subannular space.

148. The cardiac assembly according to claim 145, wherein the ventricular anchor is configured to be atraumatic so as not to penetrate tissue of surrounding anatomy.

149. The cardiac assembly according to claim 145, wherein the ventricular anchor is configured to penetrate tissue of surrounding anatomy.

150. The cardiac assembly according to claim 145, wherein the cardiac assembly does not comprise any elements that are configured to penetrate tissue.

151. The cardiac assembly according to claim 145, wherein the ventricular anchor and the cardiac treatment device comprise separate pieces that are configured to be delivered separately to the heart and to be coupled together in the heart.

152. The cardiac assembly according to claim 145, wherein the ventricular anchor and the cardiac treatment device are coupled to each other and are configured to be delivered to the heart while coupled together.

153. The cardiac assembly according to any one of claims 145-152, wherein the atrial-surface support comprises a frame.

154. The cardiac assembly according to claim 153, wherein the frame defines at least a portion of a border of the atrial-surface support, and wherein the proximal supra-leaflet support is coupled to two lateral sides of the frame.

155. The cardiac assembly according to claim 153, wherein the atrial-surface support comprises an atrial-surface cover, which is coupled to the frame.

156. The cardiac assembly according to claim 155, wherein the atrial-surface cover is in contact with the proximal supra-leaflet support in addition to being coupled to the frame.

157. The cardiac assembly according to claim 155, wherein the atrial-surface cover is coupled to a surface of the frame that faces the proximal subannular anchor.

158. The cardiac assembly according to any one of claims 145-152, wherein the proximal supra-leaflet support extends more proximally than a proximal-most point of the proximal subannular anchor.

159. The cardiac assembly according to claim 158, wherein one or more proximal-most portions of the proximal supra-leaflet support are bent toward the proximal subannular anchor.

160. The cardiac assembly according to claim 159, wherein the one or more proximal-most portions of the proximal supra-leaflet support are bent toward the proximal subannular anchor by an angle of at least 15 degrees with respect to more distal portions of the atrial-surface support.

161. The cardiac assembly according to any one of claims 145-152, wherein the proximal supra-leaflet support comprises a frame that comprises a zig-zag shaped wire.

162. The cardiac assembly according to claim 161, wherein the zig-zag shaped wire is shaped so as to define at least two proximal peaks.

163. The cardiac assembly according to any one of claims 145-152, wherein the cardiac treatment device comprises a neo-leaflet, which is supported by the ventricular anchor and is configured to at least partially replace function of a target native leaflet of the native atrioventricular valve by providing a surface of coaptation for one or more opposing native leaflets that oppose the target native leaflet, when anchored in place by the ventricular anchor.

164. The cardiac assembly according to claim 163, wherein the neo-leaflet is configured such that the coaptation surface is generally static throughout a cardiac cycle of the heart upon implantation of the cardiac assembly in the heart.

165. The cardiac assembly according to claim 163, wherein the neo-leaflet is configured such that the coaptation surface moves toward and away from the one or more opposing native leaflets during a cardiac cycle of the heart upon implantation of the cardiac assembly in the heart.

166. The cardiac assembly according to claim 163, wherein the coaptation surface has an area of between 2 and 20 cm2.

167. The cardiac assembly according to claim 163,
wherein the cardiac assembly comprises at least first and second wires,
wherein the first wire is shaped so as to define the proximal supra-leaflet support and a proximal first portion of a border of the neo-leaflet, and
wherein the second wire is shaped so as to define a second portion of the border of the neo-leaflet.

168. The cardiac assembly according to claim 167, wherein the first wire is thicker than the second wire.

169. The cardiac assembly according to claim 168, wherein a cross-sectional area of the first wire equals at least 150% of a cross-sectional area of the second wire.

170. The cardiac assembly according to claim 168, wherein the second wire is shaped so as to additionally define at least a portion of the ventricular anchor.

171. The cardiac assembly according to claim 168,
wherein the cardiac assembly comprises a third wire, which is shaped so as to define at least a portion of the ventricular anchor, and
wherein the first wire is thicker than the third wire.

172. The cardiac assembly according to claim 167, wherein the neo-leaflet comprises a neo-leaflet cover, which is attached to the proximal first portion and the second portion of the border of the neo-leaflet and provides the surface of coaptation.

173. The cardiac assembly according to claim 163, wherein the neo-leaflet comprises:
a neo-leaflet wire loop that defines at least a portion of a border of the neo-leaflet; and
a neo-leaflet cover, which is attached to the neo-leaflet wire loop and provides the surface of coaptation.

174. The cardiac assembly according to claim 173, wherein a width of the proximal supra-leaflet support is less than a width of the neo-leaflet wire loop.

175. The cardiac assembly according to claim 174, wherein the width of the proximal supra-leaflet support is less than 80% of the width of the neo-leaflet wire loop.

176. The cardiac assembly according to claim 173,
wherein the neo-leaflet has a central longitudinal axis that is oriented in a proximal-distal direction, and
wherein the neo-leaflet wire loop is shaped so as to define two lateral sides of the neo-leaflet that face away from the central longitudinal axis and are shaped so as to define respective curved portions having convex sides that face toward the central longitudinal axis.

177. The cardiac assembly according to claim 176, wherein the two lateral sides are not shaped so as to further define any curved portions farther from a junction between the neo-leaflet and the ventricular anchor than the respective curved portions are from the junction.

178. The cardiac assembly according to claim 176, wherein the respective curved portions are first respective curved portions, and wherein the two lateral sides are shaped so as to further define respective second curved portions having concave sides that face toward the central longitudinal axis, the second curved portions closer to a junction between the neo-leaflet and the ventricular anchor than the first curved portions are to the junction.

179. The cardiac assembly according to claim 176, wherein the respective curved portions have respective radii of curvature, each of which is between 0.25 and 5 cm.

180. The cardiac assembly according to claim 173, wherein the neo-leaflet wire loop narrows toward a junction between the neo-leaflet and the ventricular anchor.

181. The cardiac assembly according to claim 163, wherein the cardiac assembly is configured such that the coaptation surface of the neo-leaflet crosses from an atrial side to a ventricular side of a native valvular plane, when anchored in place by the ventricular anchor.

182. The cardiac assembly according to any one of claims 145-152, wherein the cardiac treatment device comprises a prosthetic heart valve, which is configured to replace the function of the native atrioventricular valve, and which comprises a frame and a plurality of prosthetic leaflets that are coupled to the frame and are configured to coapt with one another during each cardiac cycle.

183. The cardiac assembly according to any one of claims 145-152, wherein the cardiac treatment device comprises a spacer, which is configured to improve the function of the native atrioventricular valve by interacting with at least a portion of at least one leaflet of the native atrioventricular valve so as to at least partially restrict blood flow through the native atrioventricular valve when the native atrioventricular valve is closed during each cardiac cycle.

184. The cardiac assembly according to any one of claims 145-152, wherein the ventricular anchor comprises one or more wires, and wherein the distal anchor is defined at least in part by at least one of the one or more wires shaped as a distal wire loop.

185. The cardiac assembly according to claim 184, wherein two longitudinal portions of the at least one of the one or more wires that defines the distal anchor are fixed to each other.

186. The cardiac assembly according to claim 185, wherein the two longitudinal portions are fixed to each other by crimping.

187. The cardiac assembly according to claim 186, wherein the two longitudinal portions are crimped together by a tube.

188. The cardiac assembly according to claim 184, wherein the distal wire loop is configured to be positioned in the ventricle, extending to a ventricular apical area.

189. The cardiac assembly according to claim 188, wherein the distal wire loop is configured to remain anchored in position against the ventricular wall and one or more other ventricular structures of the ventricular apical area, when the distal anchor is positioned partially against the ventricular wall outside the target subannular space.

190. The cardiac assembly according to claim 189, wherein the one or more other ventricular structures include one or more structures selected from the group consisting of: one or more ventricular papillary muscles, and a moderator band.

191. The cardiac assembly according to claim 188, wherein the distal wire loop is shaped so as to define three or more lobes.

192. The cardiac assembly according to claim 188, wherein the distal wire loop is configured to remain anchored in position by force applied by the distal wire loop to surrounding anatomy.

193. The cardiac assembly according to claim 188, wherein the distal wire loop is configured to remain anchored in position by friction between the distal wire loop and surrounding anatomy.

194. The cardiac assembly according to claim 184, wherein the distal anchor comprises a covering that covers at least a portion of the distal wire loop.

195. The cardiac assembly according to claim 194, wherein the covering comprises a coating that coats the at least a portion of the distal wire loop.

196. The cardiac assembly according to claim 194, wherein the covering comprises a sheet of material that covers the at least a portion of the distal wire loop.

197. The cardiac assembly according to claim 196, wherein the covering entirely covers the distal wire loop.

198. The cardiac assembly according to claim 196, wherein the covering partially covers the distal wire loop.

199. The cardiac assembly according to claim 198, wherein the distal wire loop is shaped so as to define two or more lobes, and wherein the anchor covering does not cover any portion of the lobes.

200. The cardiac assembly according to claim 198, wherein the distal wire loop is shaped so as to define two or more lobes, and wherein the anchor covering covers only respective portions of the lobes.

201. The cardiac assembly according to claim 198, wherein the distal wire loop is shaped so as to define two or more lobes, and wherein the anchor covering entirely covers the lobes.

202. The cardiac assembly according to claim 194,
wherein the one or more wires that are shaped as the distal wire loop are one or more primary wires, and
wherein the covering comprises a secondary wire that is coiled around the one or more primary wires.

203. The cardiac assembly according to any one of claims 145-152, wherein the ventricular anchor comprises one or more wires, and wherein a continuous portion of the one or more wires is shaped so as to define both the distal anchor and at least a portion of the proximal subannular anchor.

204. The cardiac assembly according to claim 203, wherein the portion of the at least one of the one or more wires that defines the distal anchor is shaped as a distal wire loop.

205. The cardiac assembly according to any one of claims 145-152, wherein the proximal subannular anchor is configured such that, when the proximal subannular anchor is positioned at least partially in the target subannular space, the proximal subannular anchor applies a force to a ventricular surface of the target native leaflet, so as to help anchor the ventricular anchor to the target native leaflet.

206. The cardiac assembly according to any one of claims 145-152, wherein the native atrioventricular valve is a tricuspid valve, and wherein the cardiac treatment device is configured to replace or improve function of the tricuspid valve, when anchored in place by the ventricular anchor.

207. The cardiac assembly according to claim 206, wherein the target native leaflet is a native septal leaflet of the tricuspid valve, and wherein the ventricular wall is a ventricular septal wall.

208. The cardiac assembly according to any one of claims 145-152, wherein the native atrioventricular valve is a mitral valve, and wherein the cardiac treatment device is configured to replace or improve function of the mitral valve, when anchored in place by the ventricular anchor.

209. The cardiac assembly according to claim 208, wherein the target native leaflet is a native anterior leaflet of the mitral valve, and wherein the ventricular wall is a ventricular septal wall.

210. The cardiac assembly according to any one of claims 145-152, further comprising at least one pouch, which is configured to inflate by blood flow during a cardiac cycle of the heart, so as to push the cardiac assembly against one or more of: a ventricular surface of the target native leaflet and an annulus of the native atrioventricular valve, thereby stabilizing the cardiac assembly with respect to the native atrioventricular valve.

211. The cardiac assembly according to any one of claims 145-152, wherein the proximal subannular anchor generally defines a curved surface having a concave side that faces toward the cardiac treatment device.

212. The cardiac assembly according to claim 211,
wherein the ventricular anchor comprises one or more wires, and
wherein the proximal subannular anchor (a) comprises a digitate anchor that (i) is defined at least in part by at least one of the one or more wires, and (ii) is shaped so as to define a plurality of fingers having a plurality of curved superior peaks, and (b) is configured to be positioned at least partially in the target subannular space such that (i) the curved superior peaks point in a superior direction and engage a subannular surface of the target native leaflet, and (ii) one or more of the fingers engage chordae tendineae that extend between the target native leaflet and the superior portion of the ventricular wall, so as to stabilize the cardiac assembly.

213. The cardiac assembly according to claim 212, wherein the digitate anchor further comprises a digitate cover, which is coupled to one or more of the fingers, and is digitate-shaped like the fingers.

214. The cardiac assembly according to any one of claims 145-152,
wherein the cardiac assembly is shaped so as to define a junction between the proximal subannular anchor and the atrial-surface support, and
wherein the cardiac assembly is configured such that the proximal subannular anchor swings outward away from the atrial-surface support upon application of a distally-directed force to the junction while the atrial-surface support is held axially stationary.

215. A system comprising the cardiac assembly according to any one of claims 145-152, the system further comprising a delivery tube in which the cardiac assembly is removably disposed in a compressed configuration for delivery to the heart.

216. A cardiac assembly for application to a native atrioventricular valve of a heart, the cardiac assembly comprising:
a ventricular anchor, which is configured to be positioned in a ventricle of the heart, and which comprises:
(i) a proximal subannular anchor, which is configured to be positioned at least partially in a target subannular space defined by the target native leaflet and a superior portion of a ventricular wall of the ventricle; and
(ii) a distal anchor, which is configured to be positioned partially against the ventricular wall outside the target subannular space, so as to push on the proximal subannular anchor to help anchor the proximal subannular anchor in place within the target subannular space;
a cardiac treatment device, which is configured to replace or improve function of the native atrioventricular valve; and
one or more proximal loops.

217. The cardiac assembly according to claim 216, wherein the cardiac assembly comprises two or more proximal loops.

218. The cardiac assembly according to claim 216, wherein the one or more proximal loops have a greatest outer dimension of between 2 and 10 mm.

219. The cardiac assembly according to claim 216, wherein the one or more proximal loops are elliptical or circular.

220. The cardiac assembly according to claim 216, wherein each of the one or more proximal loops is shaped so as to define at least two turns.

221. The cardiac assembly according to claim 216, wherein the distal anchor is configured to contact the ventricular wall inferior to the target subannular space.

222. The cardiac assembly according to claim 216, wherein the ventricular anchor is configured to be atraumatic so as not to penetrate tissue of surrounding anatomy.

223. The cardiac assembly according to claim 216, wherein the ventricular anchor is configured to penetrate tissue of surrounding anatomy.

224. The cardiac assembly according to claim 216, wherein the cardiac assembly does not comprise any elements that are configured to penetrate tissue.

225. The cardiac assembly according to claim 216, wherein the ventricular anchor and the cardiac treatment device comprise separate pieces that are configured to be delivered separately to the heart and to be coupled together in the heart.

226. The cardiac assembly according to claim 216, wherein the ventricular anchor and the cardiac treatment device are coupled to each other and are configured to be delivered to the heart while coupled together.

227. The cardiac assembly according to any one of claims 216-226, wherein the cardiac treatment device comprises a neo-leaflet, which is supported by the ventricular anchor and is configured to at least partially replace function of a target native leaflet of the native atrioventricular valve by providing a surface of coaptation for one or more opposing native leaflets that oppose the target native leaflet, when anchored in place by the ventricular anchor.

228. The cardiac assembly according to claim 227, wherein the one or more proximal loops are located at a junction between the neo-leaflet and the ventricular anchor.

229. The cardiac assembly according to claim 227, wherein the neo-leaflet is configured such that the coaptation surface is generally static throughout a cardiac cycle of the heart upon implantation of the cardiac assembly in the heart.

230. The cardiac assembly according to claim 227, wherein the neo-leaflet is configured such that the coaptation surface moves toward and away from the one or more opposing native leaflets during a cardiac cycle of the heart upon implantation of the cardiac assembly in the heart.

231. The cardiac assembly according to claim 227, wherein the coaptation surface has an area of between 2 and 20 cm2.

232. The cardiac assembly according to claim 227, wherein the cardiac assembly comprises a wire that is shaped so as to define the one or more proximal loops, at least a portion of the ventricular anchor, and at least a portion of a border of the neo-leaflet.

233. The cardiac assembly according to claim 227, wherein the cardiac assembly is configured such that the coaptation surface of the neo-leaflet crosses from an atrial side to a ventricular side of a native valvular plane, when anchored in place by the ventricular anchor.

234. The cardiac assembly according to claim 232, wherein the one or more proximal loops are located at a junction between the neo-leaflet and the ventricular anchor.

235. The cardiac assembly according to claim 227, wherein the neo-leaflet comprises:
a neo-leaflet wire loop that defines at least a portion of a border of the neo-leaflet; and
a neo-leaflet cover, which is attached to the neo-leaflet wire loop and provides the surface of coaptation.

236. The cardiac assembly according to claim 235,
wherein the neo-leaflet has a central longitudinal axis that is oriented in a proximal-distal direction, and
wherein the neo-leaflet wire loop is shaped so as to define two lateral sides of the neo-leaflet that face away from the central longitudinal axis and are shaped so as to define respective curved portions having convex sides that face toward the central longitudinal axis.

237. The cardiac assembly according to claim 236, wherein the two lateral sides are not shaped so as to further define any curved portions farther from a junction between the neo-leaflet and the ventricular anchor than the respective curved portions are from the junction.

238. The cardiac assembly according to claim 236, wherein the respective curved portions are first respective curved portions, and wherein the two lateral sides are shaped so as to further define respective second curved portions having concave sides that face toward the central longitudinal axis, the second curved portions closer to a junction between the neo-leaflet and the ventricular anchor than the first curved portions are to the junction.

239. The cardiac assembly according to claim 236, wherein the respective curved portions have respective radii of curvature, each of which is between 0.25 and 5 cm.

240. The cardiac assembly according to claim 235, wherein the neo-leaflet wire loop narrows toward a junction between the neo-leaflet and the ventricular anchor.

241. The cardiac assembly according to any one of claims 216-226, wherein the cardiac treatment device comprises a prosthetic heart valve, which is configured to replace the function of the native atrioventricular valve, and which comprises a frame and a plurality of prosthetic leaflets that are coupled to the frame and are configured to coapt with one another during each cardiac cycle.

242. The cardiac assembly according to any one of claims 216-226, wherein the cardiac treatment device comprises a spacer, which is configured to improve the function of the native atrioventricular valve by interacting with at least a portion of at least one leaflet of the native atrioventricular valve so as to at least partially restrict blood flow through the native atrioventricular valve when the native atrioventricular valve is closed during each cardiac cycle.

243. The cardiac assembly according to any one of claims 216-226, wherein the ventricular anchor comprises one or more wires, and wherein the distal anchor is defined at least in part by at least one of the one or more wires shaped as a distal wire loop.

244. The cardiac assembly according to claim 243, wherein two longitudinal portions of the at least one of the one or more wires that defines the distal anchor are fixed to each other.

245. The cardiac assembly according to claim 244, wherein the two longitudinal portions are fixed to each other by crimping.

246. The cardiac assembly according to claim 245, wherein the two longitudinal portions are crimped together by a tube.

247. The cardiac assembly according to claim 243, wherein the distal wire loop is configured to be positioned in the ventricle, extending to a ventricular apical area.

248. The cardiac assembly according to claim 247, wherein the distal wire loop is configured to remain anchored in position against the ventricular wall and one or more other ventricular structures of the ventricular apical area, when the distal anchor is positioned partially against the ventricular wall outside the target subannular space.

249. The cardiac assembly according to claim 248, wherein the one or more other ventricular structures include one or more structures selected from the group consisting of: one or more ventricular papillary muscles, and a moderator band.

250. The cardiac assembly according to claim 247, wherein the distal wire loop is shaped so as to define three or more lobes.

251. The cardiac assembly according to claim 247, wherein the distal wire loop is configured to remain anchored in position by force applied by the distal wire loop to surrounding anatomy.

252. The cardiac assembly according to claim 247, wherein the distal wire loop is configured to remain anchored in position by friction between the distal wire loop and surrounding anatomy.

253. The cardiac assembly according to claim 243, wherein the distal anchor comprises a covering that covers at least a portion of the distal wire loop.

254. The cardiac assembly according to claim 27, wherein the covering comprises a coating that coats the at least a portion of the distal wire loop.

255. The cardiac assembly according to claim 253, wherein the covering comprises a sheet of material that covers the at least a portion of the distal wire loop.

256. The cardiac assembly according to claim 255, wherein the covering entirely covers the distal wire loop.

257. The cardiac assembly according to claim 255, wherein the covering partially covers the distal wire loop.

258. The cardiac assembly according to claim 257, wherein the distal wire loop is shaped so as to define two or more lobes, and wherein the anchor covering does not cover any portion of the lobes.

259. The cardiac assembly according to claim 257, wherein the distal wire loop is shaped so as to define two or more lobes, and wherein the anchor covering covers only respective portions of the lobes.

260. The cardiac assembly according to claim 257, wherein the distal wire loop is shaped so as to define two or more lobes, and wherein the anchor covering entirely covers the lobes.

261. The cardiac assembly according to claim 253,
wherein the one or more wires that are shaped as the distal wire loop are one or more primary wires, and
wherein the covering comprises a secondary wire that is coiled around the one or more primary wires.

262. The cardiac assembly according to any one of claims 216-226, wherein the ventricular anchor comprises one or more wires, and wherein a continuous portion of the one or more wires is shaped so as to define both the distal anchor and at least a portion of the proximal subannular anchor.

263. The cardiac assembly according to claim 262, wherein the portion of the at least one of the one or more wires that defines the distal anchor is shaped as a distal wire loop.

264. The cardiac assembly according to any one of claims 216-226, wherein the proximal subannular anchor is configured such that, when the proximal subannular anchor is positioned at least partially in the target subannular space, the proximal subannular anchor applies a force to a ventricular surface of the target native leaflet, so as to help anchor the ventricular anchor to the target native leaflet.

265. The cardiac assembly according to any one of claims 216-226, wherein the native atrioventricular valve is a tricuspid valve, and wherein the cardiac treatment device is configured to replace or improve function of the tricuspid valve, when anchored in place by the ventricular anchor.

266. The cardiac assembly according to claim 265, wherein the target native leaflet is a native septal leaflet of the tricuspid valve, and wherein the ventricular wall is a ventricular septal wall.

267. The cardiac assembly according to any one of claims 216-226, wherein the native atrioventricular valve is a mitral valve, and wherein the cardiac treatment device is configured to replace or improve function of the mitral valve.

268. The cardiac assembly according to claim 267, wherein the target native leaflet is a native anterior leaflet of the mitral valve, and wherein the ventricular wall is a ventricular septal wall.

269. The cardiac assembly according to any one of claims 216-226, further comprising at least one pouch, which is configured to inflate by blood flow during a cardiac cycle of the heart, so as to push the cardiac assembly against one or more of: a ventricular surface of the target native leaflet and an annulus of the native atrioventricular valve, thereby stabilizing the cardiac assembly with respect to the native atrioventricular valve.

270. The cardiac assembly according to any one of claims 216-226, wherein the proximal subannular anchor generally defines a curved surface having a concave side that faces toward the cardiac treatment device.

271. The cardiac assembly according to claim 270,
wherein the ventricular anchor comprises one or more wires, and
wherein the proximal subannular anchor (a) comprises a digitate anchor that (i) is defined at least in part by at least one of the one or more wires, and (ii) is shaped so as to define a plurality of fingers having a plurality of curved superior peaks, and (b) is configured to be positioned at least partially in the target subannular space such that (i) the curved superior peaks point in a superior direction and engage a subannular surface of the target native leaflet, and (ii) one or more of the fingers engage chordae tendineae that extend between the target native leaflet and the superior portion of the ventricular wall, so as to stabilize the cardiac assembly.

272. The cardiac assembly according to claim 271, wherein the digitate anchor further comprises a digitate cover, which is coupled to one or more of the fingers, and is digitate-shaped like the fingers.

273. The cardiac assembly according to any one of claims 216-226, further comprising an atrial-surface support, which is configured to be disposed below or at a level of an annulus of the native atrioventricular valve and against an atrial surface of the target native leaflet when the proximal subannular anchor is positioned at least partially in the target subannular space.

274. The cardiac assembly according to claim 273,
wherein the cardiac assembly is shaped so as to define a junction between the proximal subannular anchor and the atrial-surface support, and
wherein the cardiac assembly is configured such that the proximal subannular anchor swings outward away from the atrial-surface support upon application of a distally-directed force to the junction while the atrial-surface support is held axially stationary.

275. The cardiac assembly according to claim 274, wherein the cardiac assembly is configured such that the proximal subannular anchor swings outward away from the atrial-surface support upon application of the distally-directed force to the junction while the atrial-surface support is held axially stationary by holding at least one of the one or more proximal loops stationary.

276. A system comprising the cardiac assembly according to any one of claims 216-226, the system further comprising:
a delivery tube in which the cardiac assembly is removably disposed in a compressed configuration for delivery to the heart; and
one or more delivery elongate members, which are removably coupled to the one or more proximal loops, respectively.

277. The system according to claim 276, further comprising one or more couplers, wherein the one or more elongate delivery members are removably coupled to the one or more proximal loops, respectively, via the one or more couplers, respectively.

278. The system according to claim 276,
wherein the system is configured to deploy the cardiac assembly out of a distal end opening of the delivery tube, and
wherein the cardiac assembly is removably disposed in the delivery tube in the compressed configuration, with the cardiac treatment device and the ventricular anchor extending distally from the one or more proximal loops.
